SEQUENCE LISTING (35 sequences)

SEQ ID NO: 1 - protein, 11 aa, synthetic construct
RASQSVSSAVA

SEQ ID NO: 2 - protein, 7 aa, synthetic construct
SASSLYS

SEQ ID NO: 3 - protein, 9 aa, synthetic construct
QQSSTDPIT

SEQ ID NO: 4 - protein, 9 aa, synthetic construct
FTVSSSSIH

SEQ ID NO: 5 - protein, 17 aa, synthetic construct
YISSSSGYTYYADSVKG

SEQ ID NO: 6 - protein, 15 aa, synthetic construct
YWSYPSWWPYRGMDY

SEQ ID NO: 7 - protein, 124 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSS

SEQ ID NO: 8 - protein, 108 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSSTDPITFGQGTKVEIKR

SEQ ID NO: 9 - protein, 20 aa, synthetic construct
MYRMQLLSCIALSLALVTNS

SEQ ID NO: 10 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 106 aa, unidentified
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 14 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 15 - protein, 214 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSSTDPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 454 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 454 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 451 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 19 - protein, 451 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 20 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 451 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 451 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 27 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 28 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 29 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 30 - protein, 453 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 453 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 450 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 33 - protein, 450 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 34 - protein, 450 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 35 - protein, 450 aa, synthetic construct
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

(19) United States
(12) Patent Application Publication
(10) Pub. No.: US 2026/0226175 A1
(43) Pub. Date: Aug. 6, 2026

(54) TREATMENT OF HEMATOLOGICAL MALIGNANCIES WITH ANTIBODIES INHIBITING GALECTIN-9

(71) Applicant: PureTech LYT, Inc., Boston, MA (US)

(72) Inventors: Aleksandra FILIPOVIC, Boston, MA (US); Christopher C. KORTH, Boston, MA (US); Julie Swartzman KROP, Boston, MA (US)

(21) Appl. No.: 19/651,261

(22) Filed: Apr. 17, 2026

Related U.S. Application Data
(63) Continuation-in-part of application No. PCT/US2024/052038, filed on Oct. 18, 2024.
(60) Provisional application No. 63/591,589, filed on Oct. 19, 2023.
(60) Provisional application No. 63/562,397, filed on Mar. 7, 2024.

(51) Int. Cl. C07K 16/28; A61K 9/00; A61K 31/496; A61K 31/706; A61K 39/00; A61P 35/02

(52) U.S. Cl. CPC C07K 16/2851; A61K 9/0019; A61K 31/496; A61K 31/706; A61P 35/02; A61K 2039/505; A61K 2039/54; A61K 2039/545; C07K 2317/52; C07K 2317/565

(57) ABSTRACT

Disclosed herein are combined therapies for treating a hematologic malignancy (e.g., acute myeloid leukemia (AML), or myelodysplastic syndromes (MDS)), using an antibody that binds human galectin-9 (anti-Gal9 antibody, e.g., G9.2-17), and one or more chemotherapeutics, e.g., a Bcl2 inhibitor such as venetoclax, a hypomethylating agent (HMA), or a combination thereof.

### CROSS REFERENCE TO RELATED APPLICATIONS

This application is a continuation-in-part application of International Application No. PCT/US2024/052038, filed Oct. 18, 2024, which claims the benefit of the filing dates of U.S. Provisional Application No. 63/562,397, filed Mar. 7, 2024, and U.S. Provisional Application No. 63/591,589, filed Oct. 19, 2023. The entire contents of each of these priority applications are incorporated by reference herein.

### SEQUENCE LISTING

The instant application contains a Sequence Listing which has been filed electronically in XML format and is hereby incorporated by reference in its entirety. Said XML copy, created on Apr. 16, 2026, is named “063587-515002US_Seq-Listing_ST26.xml” and is 40,993 bytes in size.

### BACKGROUND OF INVENTION

Galectin-9 is a tandem-repeat lectin consisting of two carbohydrate recognition domains (CRDs) and was discovered and described for the first time in 1997 in patients suffering from Hodgkin's lymphoma (HL) (Tureci et al., J. Biol. Chem. 1997, 272, 6416-6422). Three isoforms exist and can be located within the cell or extracellularly. Elevated Galectin-9 levels have been observed in a wide range of cancers, including melanoma, Hodgkin's lymphoma, hepatocellular, pancreatic, gastric, colon and clear cell renal cell cancers (Wdowiak et al. Int. J. Mol. Sci. 2018, 19, 210). In renal cancer, patients with high Galectin-9 expression showed more advanced progression of the disease with larger tumor size (Kawashima et al.; BJU Int. 2014; 113:320-332). In melanoma, Galectin-9 was expressed in 57% of tumors and was significantly increased in the plasma of patients with advanced melanoma compared to healthy controls (Enninga et al., Melanoma Res. 2016 October; 26(5): 429-441). A number of studies have shown utility for Galectin-9 as a prognostic marker, and more recently as a potential new drug target (Enninga et al., 2016; Kawashima et al. BJU Int 2014; 113: 320-332; Kageshita et al., Int J Cancer. 2002 Jun. 20; 99(6):809-16, and references therein).

Galectin-9 has been described to play an important role in in a number of cellular processes such as adhesion, cancer cell aggregation, apoptosis, and chemotaxis. Recent studies have shown a role for Galectin-9 in immune modulation in support of the tumor, e.g., through negative regulation of Th1 type responses, Th2 polarization and polarization of macrophages to the M2 phenotype. This work also includes studies that have shown that Galectin-9 participates in direct inactivation of T cells through interactions with the T-cell immunoglobulin and mucin protein 3 (TIM-3) receptor (Dardalhon et al., J Immunol., 2010, 185, 1383-1392; Sanchez-Fueyo et al., Nat Immunol., 2003, 4, 1093-1101).

Galectin-9 has also been found to play a role in polarizing T cell differentiation into tumor suppressive phenotypes), as well as promoting tolerogenic macrophage programming and adaptive immune suppression (Daley et al., Nat Med., 2017, 23, 556-567). In mouse models of pancreatic ductal adenocarcinoma (PDAC), blockade of the checkpoint interaction between Galectin-9 and the receptor Dectin-1 found on innate immune cells in the tumor microenvironment (TME) has been shown to increase anti-tumor immune responses in the TME and to slow tumor progression (Daley et al., Nat Med., 2017, 23, 556-567). Galectin-9 also has been found to bind to CD206, a surface marker of M2 type macrophages, resulting in a reduced secretion of CVL22 (MDC), a macrophage derived chemokine which has been associated with longer survival and lower recurrence risk in lung cancer (Enninga et al, J Pathol. 2018 August; 245(4):468-477).

### SUMMARY OF INVENTION

The present disclosure is based, at least in part, on the development of treatment regimen for hematological malignancies such as acute myeloid leukemia (AML) and myelodysplastic syndromes (MDS).

Accordingly, provided herein is a method for treating a hematologic malignancy, comprising administering to a subject in need thereof an antibody that binds human galectin-9 (anti-Gal9 antibody), and one or more chemotherapeutics such as venetoclax, a hypomethylating agent, or a combination thereof. The anti-Gal9 antibody comprises: (a) a light chain comprising a light chain variable region (V_{L}), which comprises a light chain (LC) complementarity determining region 1 (CDR1) comprising the amino acid sequence of SEQ ID NO: 1, a LC complementarity determining region 2 (CDR2) comprising the amino acid sequence of SEQ ID NO: 2, and a LC complementarity determining region 3 (CDR3) comprising the amino acid sequence of SEQ ID NO: 3 and (b) a heavy chain comprising a heavy chain variable region (V_{H}), which comprises a heavy chain (HC) complementarity determining region 1 (CDR1) comprising the amino acid sequence of SEQ ID NO: 4, a HC complementarity determining region 2 (CDR2) comprising the amino acid sequence of SEQ ID NO: 5, and a HC complementarity determining region 3 (CDR3) comprising the amino acid sequence of SEQ ID NO: 6.

In some embodiments, the anti-Gal9 antibody may be administered to the subject at a dose of about 2 mg/kg to about 16 mg/kg once every week (QW) by intravenous infusion. In one example, the anti-Gal9 antibody disclosed herein (e.g., G9.2-17 (IgG4)) may be administered to the subject at a dose of about 2 mg/kg to about 4 mg/kg once every week by intravenous infusion (e.g., about 2 mg/kg, about 3 mg/kg, about 4 mg/kg, or any increment therein). In another example, the anti-Gal9 antibody disclosed herein (e.g., G9.2-17 (IgG4)) may be administered to the subject at a dose of about 4 mg/kg to 7.5 mg/kg once every week by intravenous infusion (e.g., about 4 mg/kg, about 5 mg/kg, about 6 mg/kg, about 7.5 mg/kg, or any increment therein). In still another example, the anti-Gal9 antibody disclosed herein (e.g., G9.2-17 (IgG4)) may be administered to the subject at a dose of about 7.5 mg/kg to 12 mg/kg once every week by intravenous infusion (e.g., about 7.5 mg/kg, about 8 mg/kg, about 9 mg/kg, about 10 mg/kg, about 11 mg/kg, about 12 mg/kg, or any increment therein). In yet another example, the anti-Gal9 antibody disclosed herein (e.g., G9.2-17 (IgG4)) may be administered to the subject at a dose of about 12 mg/kg to about 16 mg/kg once every week by intravenous infusion (e.g., about 12 mg/kg, about 13 mg/kg, about 14 mg/kg, about 15 mg/kg, about 16 mg/kg, or any increment therein). In some specific examples, the anti-Gal9 antibody may be administered to the subject at a dose of about 2 mg/kg, about 4 mg/kg, about 7.5 mg/kg, about 12 mg/kg, or about 16 mg/kg once every week by intravenous infusion. In a specific example, the anti-Gal9 antibody disclosed herein (e.g., G9.2-17 (IgG4)) may be administered to the subject at a dose of about 12 mg/kg once every week.

Alternatively, the anti-Gal9 antibody may be administered to the subject at a dose of about 2 mg/kg to about 16 mg/kg once every 2 weeks (Q2W) by intravenous infusion. In one example, the anti-Gal9 antibody disclosed herein (e.g., G9.2-17 (IgG4)) may be administered to the subject at a dose of about 2 mg/kg to about 4 mg/kg once every 2 weeks by intravenous infusion (e.g., about 2 mg/kg, about 3 mg/kg, about 4 mg/kg, or any increment therein). In another example, the anti-Gal9 antibody disclosed herein (e.g., G9.2-17 (IgG4)) may be administered to the subject at a dose of about 4 mg/kg to 7.5 mg/kg once every 2 weeks by intravenous infusion (e.g., about 4 mg/kg, about 5 mg/kg, about 6 mg/kg, about 7.5 mg/kg, or any increment therein). In still another example, the anti-Gal9 antibody disclosed herein (e.g., G9.2-17 (IgG4)) may be administered to the subject at a dose of about 7.5 mg/kg to 12 mg/kg once every 2 weeks by intravenous infusion (e.g., about 7.5 mg/kg, about 8 mg/kg, about 9 mg/kg, about 10 mg/kg, about 11 mg/kg, about 12 mg/kg, or any increment therein). In yet another example, the anti-Gal9 antibody disclosed herein (e.g., G9.2-17 (IgG4)) may be administered to the subject at a dose of about 12 mg/kg to about 16 mg/kg once every 2 weeks by intravenous infusion (e.g., about 12 mg/kg, about 13 mg/kg, about 14 mg/kg, about 15 mg/kg, about 16 mg/kg, or any increment therein). In some specific examples, the anti-Gal9 antibody may be administered to the subject at a dose of about 2 mg/kg, about 4 mg/kg, about 7.5 mg/kg, about 12 mg/kg, or about 16 mg/kg once every 2 weeks by intravenous infusion.

In some embodiments, the subject for treatment by any of the methods disclosed herein is a human patient having a leukemia or lymphoma. In some instances, the hematologic malignancy is refractory and/or relapsed.

In some instances, the human patient has acute myeloid leukemia (AML). In some specific examples, the human patient has relapsed and/or refractory AML post at least one line of prior therapy. In some instances, the human patient has a myelodysplastic syndrome. In some specific examples, the human patient has relapsed and/or refractory MDS post at least one line of prior therapy. Alternatively or in addition, the human patient does not have acute promyelocytic leukemia (APL).

In some instances, the human patient (AML patients or MDS patients) for treatment by any of the methods disclosed herein may carry one or more biomarkers, for example, a mutation in at least one of the following genes: ABL1, ASXL1, BCOR, BCORL1, BRAF, CBFBMYH1, CBL, CBLB, CEBPA, CSF3R, CUX1, DDX41, DNMT3A, EZH2, GATA2, IDH2, JAK2, KRAS, MPL, MUTYH, NF1, NPM1, NRAS, NSD1, p53, PHF6, PMLRARA, PD_1, RAD21, RUNX1, RUNX1T1; SETBP1, SF3B1, SRFS2, SRSF2, STAG2, TBL1XR1/MECOM fusion, TET2, U2AF1, WT1, and ZRSR2. Patients with such genetic mutations are known to be difficult to treat. The anti-Gal9 antibody, either alone or in combination with the chemotherapeutics provided herein showed promising treatment efficacy in such patients.

In some examples, the human patient who has AIL is administered the anti-Gal9 antibody, a B-cell lymphoma-2 (BCL-2) inhibitor such as venetoclax (e.g., VENCLEXTA®), and a hypomethylating agent (HMA), which is azacytidine (e.g., Vidaza®) or decitabine (e.g., DACOGEN®).

The amount of venetoclax to be given to a human patient depends on many factors, including height and weight, general health or other health problems, and the type of cancer to be treated, which would be within the knowledge of a medical practitioner following guidance provided by the Food and Drug Administration (e.g., see the drug labels of approved venetoclax products). In some examples, a human patient may be administered with venetoclax orally at a dose of 100 mg on Day 1, at 200 mg on Day 2, and at 400 mg on each day of Days 3-28 for a treatment cycle of 28 days. If one or more adverse effects occur, the dose of venetoclax may be reduced or the treatment may be withheld. More details for managing adverse effects associated with venetoclax treatment are provided in Example 1 below.

The amount of azacytidine to be given to a human patient depends on many factors, including height and weight, general health or other health problems, and the type of cancer to be treated, which would be within the knowledge of a medical practitioner following guidance provided by the Food and Drug Administration (e.g., see the drug labels of approved azacytidine products). In some examples, a human patient may be administered with azacytidine subcutaneously or intravenously at a dose of 75 mg/m² on each of Days 1-7 for a treatment cycle of 28 days. In some specific examples, a human patient may be administered with azacytidine subcutaneously at a dose of 75 mg/m² on each of Days 1-7 for a treatment cycle of 28 days. If one or more adverse effects occur, the dose of azacytidine may be reduced or the treatment may be withheld. More details for managing adverse effects associated with azacytidine treatment are provided in Example 1 below.

The amount of decitabine to be given to a human patient depends on many factors, including height and weight, general health or other health problems, and the type of cancer to be treated, which would be within the knowledge of a medical practitioner following guidance provided by the Food and Drug Administration (e.g., see the drug labels of approved decitabine products). In some examples, a human patient may be administered with decitabine intravenously at a dose of 75 mg/m² on each day of Days 1-5 for a treatment cycle of 28 days. If one or more adverse effects occur, the dose of decitabine may be reduced or the treatment may be withheld. More details for managing adverse effects associated with decitabine treatment are provided in Example 1 below.

Use of azacytidine and decitabine is generally interchangeable. For patients who are resistant to or do not respond to azacytidine, decitabine can be used as an alternative. Likewise, for patients who are resistant to or do not respond to decitabine, azacytidine can be used as an alternative.

In some specific examples, the treatment regimen comprises one or more of a cycle of 28 days, wherein in each cycle: the anti-Gal9 antibody is administered to the human patient on Day 1, Day 8, Day 15, and Day 22 at a dose of about 4 mg/kg, about 7.5 mg/kg, about 12 mg/kg, or about 16 mg/kg once every week (e.g., 12 mg/kg once every week) by intravenous infusion; the venetoclax is administered to the human patient orally at 100 mg on Day 1, at 200 mg on Day 2, and at 400 mg on each day of Days 3-28; and the azacytidine at 75 mg/m² subcutaneously or intravenously on each of Days 1-7.

In other specific examples, the treatment regimen comprises one or more of a cycle of 28 days, wherein in each cycle: the anti-Gal9 antibody is administered to the human patient on Day 1, Day 8, Day 15, and Day 22 at a dose of about 4 mg/kg, about 7.5 mg/kg, about 12 mg/kg, or about 16 mg/kg once every week (e.g., 12 mg/kg once every week) by intravenous infusion; the venetoclax is administered to the human patient orally at 100 mg on Day 1, at 200 mg on Day 2, and at 400 mg on each of Days 3-28; and the decitabine at 20 mg/m2 intravenously on each day of Days 1-5. For this regimen, the administration of the decitabine is repeated every 28 days.

In any of the methods of treating AML described herein, venetoclax is administered at a reduced dose or is terminated when severe venetoclax-associated toxicity occurs.

In some instances, the human patient has myelodysplastic syndrome (MDS) and high risk or very high risk MDS. In some specifically examples, the human patient has relapsed and/or refractory MDS post at least one line of prior therapy. Alternatively or in addition, the human patient does not have acute promyelocytic leukemia (APL).

In some examples, the human patient who has MDS is administered the anti-Gal9 antibody and a hypomethylating agent (HMA), which is azacytidine or decitabine (e.g., DACOGEN®).

In some specific examples, the treatment regimen comprises one or more of a cycle of 28 days, wherein in each cycle: the anti-Gal9 antibody is administered to the human patient on Day 1, Day 8, Day 15, and Day 22 at a dose of about 4 mg/kg, about 7.5 mg/kg, about 12 mg/kg, or about 16 mg/kg once every week (e.g., 12 mg/kg once every week) by intravenous infusion; and the azacytidine at 75 mg/m² subcutaneously or intravenously on each of Days 1-7.

In other specific examples, the treatment regimen comprises one or more of a cycle of 28 days, wherein in each cycle: the anti-Gal9 antibody is administered to the human patient on Day 1, Day 8, Day 15, and Day 22 at a dose of about 4 mg/kg, about 7.5 mg/kg, about 12 mg/kg, or about 16 mg/kg once every week (e.g., 12 mg/kg once every week) by intravenous infusion; and the decitabine at 20 mg/m2 intravenously on each day of Days 1-5. For this regimen, the administration of the decitabine is repeated every 28 days.

In any of the methods of treating MDS described herein, azacytidine or decitabine is administered at a reduced dose or terminated when drug-related toxicity occurs.

For any of the treatment methods described herein, the subject is free of a treatment comprising an immunosuppressant CYP3A4 substrate, which optionally is tacrolimus, cyclosporine, everolimus, or sirolimus.

In some embodiments, the anti-Gal9 antibody used in any of the treatment methods described herein may comprise the V_{L}, which comprises the amino acid sequence of SEQ ID NO:8, and the V_{H}, which comprises the amino acid sequence of SEQ ID NO: 7.

Any of the anti-Gal9 antibodies for use in the method disclosed herein may be an IgG1 molecule. Alternatively, the antibody may be an IgG4 molecule. In some embodiments, the anti-Gal9 antibody can be an IgG4 molecule (e.g., a human IgG4 molecule) comprising a modified Fc region relative to the wild-type IgG4 counterpart (e.g., the wild-type human IgG4 counterpart). In some examples, the modified Fc region comprises the amino acid sequence of SEQ ID NO: 14. In one example, the anti-Gal9 antibody comprises a heavy chain, which comprises the amino acid sequence of SEQ ID NO: 19, and a light chain, which comprises the amino acid sequence of SEQ ID NO: 15. Such an anti-Gal9 antibody may be G9.2-17 (IgG4).

Any of the methods disclosed herein may effectively reduce or eliminate Gal9+ hematological cancer cells, for example, leukemia or lymphoma cells. Alternatively or in addition, the method disclosed herein may effectively modulate immune responses targeting the cancer cells.

Any of the methods disclosed herein may further comprise monitoring occurrence of an adverse event in the course of the treatment. In some instances, the dose of the anti-Gal9 antibody may be reduced when the adverse event occurs.

Also within the scope of the present disclosure are anti-Gal9 antibody such as G9.2-17 (IgG4) and one or more chemotherapeutics such as venetoclax and/or a hypomethylating agent (e.g., those disclosed herein) for use in a method for treating hematological malignancies such as acute myeloid leukemia (AML) and myelodysplastic syndromes (MDS), as well as use of the combination of the anti-Gal9 antibody and the chemotherapeutics for manufacturing one or more medicaments for treating the hematological malignancies.

The details of one or more embodiments of the invention are set forth in the description below. Other features or advantages of the present invention are to be apparent from the following drawing and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is a diagram depicting a study schematic of using anti-Gal9 antibody G9.2-17(IgG4) (single agent), combination of G9.2-17(IgG4), venetoclax and azacytidine, and combination of G9.2-17(IgG4) and decitabine for treating hematologic malignancy such as relapsed/refractory AML and high risk MDS. RP2D=recommended phase 2 dose. PK=pharmacokinetics. PD=pharmacodynamics.

FIGS. 2A-2C include diagrams showing Kaplan-Meier analysis of overall survival rate of AML and MDS patients in the combination cohort at 12 mg/kg G9.2-17(IgG4), venetoclax and HMA (azacidatide or decitabine) in AML patients or at 12 mg/kg G9.2-17(IgG4) and HMA (azacidatide or decitabine) in MDS patients. See descriptions in Example 1 below. FIG. 1A: a chart showing overall survival of AML/MDS patients. FIG. 2B: patient number at risk over time. FIG. 2C: a table showing overall survival analysis. OS=overall survival; events=deaths; LCL=lower confidence level; UCL=upper confidence level.

### DETAILED DESCRIPTION OF INVENTION

Provided herein are methods of co-using anti-Galectin-9 antibodies, e.g., G9.2-17, and one or more chemotherapeutics such as Bcl2 inhibitors (e.g., venetoclax) or a hypomethylating agent (HMA) (e.g., azacytidine or decitabine) for hematological malignancies, for example, acute myeloid leukemia (AML) or myelodysplastic syndromes (MDS). In some embodiments, the methods disclosed herein involve specific doses and/or dosing schedules as disclosed herein. In some instances, the methods disclosed herein target specific patient populations, for example, patients who have undergone prior treatment and show disease progression through the prior treatment, or patients who are resistant (de novo or acquired) to the prior treatment.

In some embodiments, the methods disclosed herein provide specific doses and/or dosing schedules of the anti-Gal-9 antibody disclosed herein (e.g., G9.2-17(IgG4)) in combination with the chemotherapeutic agents also disclosed herein (e.g., venetoclax, azacytidine and/or decitabine), for example, about 2 mg/kg to about 16 mg/kg of the antibody once every week (e.g., about 2 mg/kg, about 4 mg/kg, about 7.5 mg/kg, about 12 mg/kg, or about 16 mg/kg once every week) or about 7.5 mg/kg to about 16 mg/kg once every week (e.g., about 7.5 mg/kg, about 12 mg/kg, or about 16 mg/kg once every week).

### I. Antibodies Binding to Galectin-9

The present disclosure provides anti-Galectin-9 antibodies, such as G9.2-17 and functional variants thereof, for use in the treatment methods disclosed herein.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody”, e.g., anti-Galectin-9 antibody, encompasses not only intact (e.g., full-length) polyclonal or monoclonal antibodies, but also antigen-binding fragments thereof (such as Fab, Fab′, F(ab′)₂, Fv), single chain (scFv), mutants thereof, fusion proteins comprising an antibody portion, humanized antibodies, chimeric antibodies, diabodies, nanobodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody, e.g., anti-Galectin-9 antibody, includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, the EU definition, the “Contact” numbering scheme, the IMGT” numbering scheme, the “AHo” numbering scheme, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; Edelman et al., Proc Natl Acad Sci USA. 1969 May; 63(1):78-85; and Almagro, J. Mol. Recognit. 17:132-143 (2004); MacCallum et al., J. Mol. Biol. 262:732-745 (1996), Lefranc M P et al., Dev Comp Immunol, 2003 January; 27(1):55-77; and Honegger A and Pluckthun A, J Mol Biol, 2001 Jun. 8; 309(3):657-70. See also hgmp.mrc.ac.uk and bioinf.org.uk/abs).

In some embodiments, the anti-Galectin-9 antibody described herein is a full-length antibody, which contains two heavy chains and two light chains, each including a variable domain and a constant domain. Alternatively, the anti-Galectin-9 antibody can be an antigen-binding fragment of a full-length antibody. Examples of binding fragments encompassed within the term “antigen-binding fragment” of a full length antibody include (i) a Fab fragment, a monovalent fragment consisting of the V_{L}, V_{H}, C_{L} and C_{H}1 domains; (ii) a F(ab′)₂ fragment, a bivalent fragment including two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) that retains functionality. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv). See e.g., Bird et al. (1988) Science 242:423-426; and Huston et al. (1988) Proc. Natl. Acad. Sci. USA 85:5879-5883.

Any of the antibodies described herein, e.g., anti-Galectin-9 antibody, can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

Reference antibody G9.2-17 refers to an antibody capable of binding to human Galectin-9 and comprises a heavy chain variable region of SEQ ID NO: 7 and a light chain variable domain of SEQ ID NO: 8, both of which are provided below. In some embodiments, the anti-Galectin-9 antibody for use in the methods disclosed herein is the G9.2-17 antibody. In some embodiments, the anti-Galectin-9 antibody for use in the methods disclosed herein is an antibody having the same heavy chain complementarity determining regions (CDRs) as reference antibody G9.2-17 and/or the same light chain complementarity determining regions as reference antibody G9.2-17. Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., bioinforg.uk/abs/).

The heavy and light chain CDRs of reference antibody G9.2-17 is provided in Table 1 below (determined using the Kabat methodology):

TABLE 1
Heavy and Light Chain CDRs of G9.2-17

| | | | |
|---|---|---|---|
| G9.2-17 | V_{L} CDR1 | RASQSVSSAVA | SEQ ID NO: 1 |
| | V_{L} CDR2 | SASSLYS | SEQ ID NO: 2 |
| | V_{L} CDR3 | QQSSTDPIT | SEQ ID NO: 3 |
| | V_{H} CDR1 | FTVSSSSIH | SEQ ID NO: 4 |
| | V_{H} CDR2 | YISSSSGYTYYADSVKG | SEQ ID NO: 5 |
| | V_{H} CDR3 | YWSYPSWWPYRGMDY | SEQ ID NO: 6 |

In some examples, the anti-Galectin-9 antibody for use in the methods disclosed herein may comprise (following the Kabat scheme) a heavy chain complementarity determining region 1 (CDR1) set forth as SEQ ID NO: 4, a heavy chain complementarity determining region 2 (CDR2) set forth as SEQ ID NO: 5, and a heavy chain complementarity determining region 3 (CDR3) set forth as SEQ ID NO: 6 and/or may comprise a light chain complementarity determining region 1 (CDR1) set forth as SEQ ID NO: 1, a light chain complementarity determining region 2 (CDR2) set forth as SEQ ID NO: 2, and a light chain complementarity determining region 3 (CDR3) set forth as SEQ ID NO: 3. The anti-Galectin-9 antibody, including the reference antibody G9.2-17, can be in any format as disclosed herein, for example, a full-length antibody or a Fab. The term “G9.2-17(IgG4)” used herein refers to a G9.2-17 antibody which is an IgG4 molecule. Likewise, the term “G9.2-17 (Fab)” refers to a G9.2-17 antibody, which is a Fab molecule.

In some embodiments, the anti-Galectin-9 antibody or binding portion thereof comprises heavy and light chain variable regions, wherein the light chain variable region CDR1, CDR2, and CDR3 amino acid sequences have at least 80% (e.g., 80%, 85%, 90%, 91%, 92%, 93%, 94%, 95%, 96%, 97%, 98%, or 99% and any increment therein) sequence identity to the light chain variable region CDR1, CDR2, and CDR3 amino acid sequences set forth in SEQ ID NOs: 1, 2, and 3, respectively. In some embodiments, the anti-Galectin-9 antibody or binding portion thereof comprises heavy and light chain variable regions, wherein the heavy chain variable region CDR1, CDR2, and CDR3 amino acid sequences have at least 80% (e.g., 80%, 85%, 90%, 91%, 92%, 93%, 94%, 95%, 96%, 97%, 98%, or 99% and any increment therein) sequence identity to the heavy chain variable region CDR1, CDR2, and CDR3 amino acid sequences set forth in SEQ ID NO: 4, 5, and 6, respectively.

Additional Galectin-9 antibodies, e.g., which bind to the CRD1 and/or CRD2 region of Galectin-9 are described in U.S. Pat. No. 10,344,091 and co-owned, co-pending U.S. patent application Ser. No. 16/173,970 and WO 2020/198390, the relevant disclosures of each of which are incorporated by reference for the subject matter and purposes referenced herein.

In some embodiments, the anti-Galectin-9 antibody disclosed herein comprises light chain CDRs that have at least 80% (e.g., 80%, 85%, 90%, 91%, 92%, 93%, 94%, 95%, 96%, 97%, 98%, or 99% and any increment therein) sequence identity, individually or collectively, as compared with the corresponding V_{L} CDRs of reference antibody G9.2-17. Alternatively or in addition, in some embodiments, the anti-Galectin-9 antibody comprises heavy chain CDRs that have at least 80% (e.g., 80%, 85%, 90%, 91%, 92%, 93%, 94%, 95%, 96%, 97%, 98%, or 99% and any increment therein) sequence identity, individually or collectively, as compared with the corresponding V_{H} CDRs of reference antibody G9.2-17.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In other embodiments, the anti-Galectin-9 antibody described herein comprises a V_{H} that comprises the HC CDR1, HC CDR2, and HC CDR3, which collectively contain up to 8 amino acid residue variations (8, 7, 6, 5, 4, 3, 2, or 1 variation(s), including additions, deletions, and/or substitutions) relative to the HC CDR1, HC CDR2, and HC CDR3 of reference antibody G9.2-17. Alternatively or in addition, in some embodiments, the anti-Galectin-9 antibody described herein comprises a V_{H} that comprises the LC CDR1, LC CDR2, and LC CDR3, which collectively contain up to 8 amino acid residue variations (8, 7, 6, 5, 4, 3, 2, or 1 variations(s) including additions, deletions, and/or substitutions) relative to the LC CDR1, LC CDR2, and LC CDR3 of reference antibody G9.2-17.

In one example, the amino acid residue variations are conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the anti-Galectin-9 antibodies disclosed herein, having the heavy chain CDRs disclosed herein, contains framework regions derived from a subclass of germline V_{H} fragment. Such germline V_{H} regions are well known in the art. See, e.g., the IMGT database (imgt.org) or at vbase2.org/vbstat.php. Examples include the IGHV1 subfamily (e.g., IGHV1-2, IGHV1-3, IGHV1-8, IGHV1-18, IGHV1-24, IGHV1-45, IGHV1-46, IGHV1-58, and IGHV1-69), the IGHV2 subfamily (e.g., IGHV2-5, IGHV2-26, and IGHV2-70), the IGHV3 subfamily (e.g., IGHV3-7, IGHV3-9, IGHV3-11, IGHV3-13, IGHV3-15, IGHV3-20, IGHV3-21, IGHV3-23, IGHV3-30, IGHV3-33, IGHV3-43, IGHV3-48, IGHV3-49, IGHV3-53, IGHV3-64, IGHV3-66, IGHV3-72, and IGHV3-73, IGHV3-74), the IGHV4 subfamily (e.g., IGHV4-4, IGHV4-28, IGHV4-31, IGHV4-34, IGHV4-39, IGHV4-59, IGHV4-61, and IGHV4-B), the IGHV subfamily (e.g., IGHV5-51, or IGHV6-1), and the IGHV7 subfamily (e.g., IGHV7-4-1).

Alternatively, or in addition, in some embodiments, the anti-Galectin-9 antibody, having the light chain CDRs disclosed herein, contains framework regions derived from a germline Vx fragment. Examples include an IGKV1 framework (e.g., IGKV1-05, IGKV1-12, IGKV1-27, IGKV1-33, or IGKV1-39), an IGKV2 framework (e.g., IGKV2-28), an IGKV3 framework (e.g., IGKV3-11, IGKV3-15, or IGKV3-20), and an IGKV4 framework (e.g., IGKV4-1). In other instances, the anti-Galectin-9 antibody comprises a light chain variable region that contains a framework derived from a germline Vλ fragment. Examples include an IGλ1 framework (e.g., IGλV1-36, IGλV1-40, IGλV1-44, IGλV1-47, IGλV1-51), an IGλ2 framework (e.g., IGλV2-8, IGλV2-11, IGλV2-14, IGλV2-18, IGλV2-23,), an IGλ3 framework (e.g., IGλV3-1, IGλV3-9, IGλV3-10, IGλV3-12, IGλV3-16, IGλV3-19, IGλV3-21, IGλV3-25, IGλV3-27,), an IGλ4 framework (e.g., IGλV4-3, IGλV4-60, IGλV4-69,), an IGλ5 framework (e.g., IGλV5-39, IGλV5-45,), an IGλ6 framework (e.g., IGλV6-57,), an IGλ7 framework (e.g., IGλV7-43, IGλV7-46,), an IGλ8 framework (e.g., IGλV8-61), an IGλ9 framework (e.g., IGλV9-49), or an IGλ10 framework (e.g., IGλV10-54).

In some embodiments, the anti-Galectin-9 antibody for use in the method disclosed herein can be an antibody having the same heavy chain variable region (V_{H}) and/or the same light chain variable region (V_{L}) as reference antibody G9.2-17, the V_{H} and V_{L} region amino acid sequences are provided below:

V_{H}:
(SEQ ID NO: 7)
EVQLVESGGGLVQPGGSLRLSCAASGFTVSSSSIHWVRQAPGKG
LEWVAYISSSSGYTYYADSVKGRETISADTSKNTAYLQMNSLRA
EDTAVYYCARYWSYPSWWPYRGMDYWGQGTLVTVSS
V_{L}:
(SEQ ID NO: 8)
DIQMTQSPSSLSASVGDRVTITCRASQSVSSAVAWYQQKPGKAP
KLLIYSASSLYSGVPSRESGSRSGTDETLTISSLQPEDFATYYC
QQSSTDPITFGQGTKVEIKR

In some embodiments, the anti-Galectin-9 antibody has at least 80% sequence identity (e.g., 80%, 85%, 90%, 91%, 92%, 93%, 94%, 95%, 96%, 97%, 98%, or 99% identity) to the heavy chain variable region of SEQ ID NO: 7. Alternatively or in addition, the anti-Galectin-9 antibody has at least 80% sequence identity (e.g., 80%, 85%, 90%, 91%, 92%, 93%, 94%, 95%, 96%, 97%, 98%, or 99% identity) to the light chain variable region of SEQ ID NO: 8.

In some instances, the anti-Galectin-9 antibody disclosed herein is a functional variant of reference antibody G9.2-17. A functional variant can be structurally similar as the reference antibody (e.g., comprising the limited number of amino acid residue variations in one or more of the heavy chain and/or light chain CDRs as G9.2-17 as disclosed herein, or the sequence identity relative to the heavy chain and/or light chain CDRs of G9.2-17, or the VH and/or VL of G9.2-17 as disclosed herein) with substantially similar binding affinity (e.g., having a KD value in the same order) to human Galectin-9.

In some embodiments, the anti-Galectin-9 antibody as described herein can bind and inhibit the activity of Galectin-9 by at least 20% (e.g., 31%, 35%, 40%, 45%, 50%, 60%, 70%, 80%, 90%, 95% or greater, including any increment therein). The apparent inhibition constant (Ki^{app} or K_{i,app}), which provides a measure of inhibitor potency, is related to the concentration of inhibitor required to reduce enzyme activity and is not dependent on enzyme concentrations. The inhibitory activity of an anti-Galectin-9 antibody described herein can be determined by routine methods known in the art.

The K_{i,}^{app} value of an antibody may be determined by measuring the inhibitory effect of different concentrations of the antibody on the extent of the reaction (e.g., enzyme activity); fitting the change in pseudo-first order rate constant (v) as a function of inhibitor concentration to the modified Morrison equation (Equation 1) yields an estimate of the apparent Ki value. For a competitive inhibitor, the Kᵢ^{app} can be obtained from the y-intercept extracted from a linear regression analysis of a plot of Kᵢ^{app} versus substrate concentration.

$$v = A \cdot \frac{( [ E ] - [ I ] - {K}_{i}^{app} ) + \sqrt{{( [ E ] - [ I ] - {K}_{i}^{app} )}^{2} + 4 [ E ] \cdot } {K}_{i}^{app}}{2} \qquad ( Equation 1 )$$

Where A is equivalent to vₒ/E, the initial velocity (vₒ) of the enzymatic reaction in the absence of inhibitor (I) divided by the total enzyme concentration (E). In some embodiments, the anti-Galectin-9 antibody described herein has a Kᵢ^{app} value of 1000, 900, 800, 700, 600, 500, 400, 300, 200, 100, 50, 40, 30, 20, 19, 18, 17, 16, 15, 14, 13, 12, 11, 10, 9, 8, 7, 6, 5 pM or less for the target antigen or antigen epitope. In some embodiments, the anti-Galectin-9 antibody has a lower Kᵢ^{app} for a first target (e.g., the CRD2 of Galectin-9) relative to a second target (e.g., CRD1 of the Galectin-9). Differences in Kᵢ^{app} (e.g., for specificity or other comparisons) can be at least 1.5, 2, 3, 4, 5, 10, 15, 20, 37.5, 50, 70, 80, 91, 100, 500, 1000, 10,000 or 10⁵ fold. In some examples, the anti-Galectin-9 antibody inhibits a first antigen (e.g., a first protein in a first conformation or mimic thereof) greater relative to a second antigen (e.g., the same first protein in a second conformation or mimic thereof, or a second protein). In some embodiments, any of the anti-Galectin-9 antibodies is further affinity matured to reduce the Ki^{app} of the antibody to the target antigen or antigenic epitope thereof.

In some embodiments, an anti-Galectin-9 antibody as described herein has a suitable binding affinity for the target antigen (e.g., Galectin-9) or antigenic epitopes thereof. As used herein, “binding affinity” refers to the apparent association constant or K_{A}. The K_{A} is the reciprocal of the dissociation constant (K_{D}). The anti-Galectin-9 antibody described herein may have a binding affinity (K_{D}) of at least 10⁻⁵, 10⁻⁶, 10⁻⁷, 10⁻⁸, 10⁻⁹, 10⁻¹⁰ M, or lower for the target antigen or antigenic epitope. An increased binding affinity corresponds to a decreased K_{D}. Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20).

These techniques can be used to measure the concentration of bound binding protein as a function of target protein concentration. Under certain conditions, the fractional concentration of bound binding protein ([Bound]/[Total]) is generally related to the concentration of total target protein ([Target]) by the following equation:

$$[ B o u n d ] / [ T o t a l ] = [ T a r g e t ] / ( Kd + [ T a r g e t ] )$$

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay. In some cases, the in vitro binding assay is indicative of in vivo activity. In other cases, the in vitro binding assay is not necessarily indicative of in vivo activity. In some cases, tight binding is beneficial, but in other cases tight binding is not as desirable in vivo, and an antibody with lower binding affinity is more desirable.

In some embodiments, the heavy chain of any of the anti-Galectin-9 antibodies as described herein further comprise a heavy chain constant region (CH) or a portion thereof (e.g., CH1, CH2, CH3, or a combination thereof). The heavy chain constant region can be of any suitable origin, e.g., human, mouse, rat, or rabbit. In one specific example, the heavy chain constant region is from a human IgG (a gamma heavy chain) of any IgG subfamily as described herein.

In some embodiments, the heavy chain constant region of the antibodies described herein comprise a single domain (e.g., CH1, CH2, or CH3) or a combination of any of the single domains, of a constant region (e.g., SEQ ID NO: 4, 5, 6). In some embodiments, the light chain constant region of the antibodies described herein comprise a single domain (e.g., CL), of a constant region. Exemplary light and heavy chain sequences are listed below. Exemplary light and heavy chain sequences are listed below. The hIgG1 LALA sequence includes two mutations, L234A and L235A (EU numbering), which suppress FcgR binding as well as a P329G mutation (EU numbering) to abolish complement C1q binding, thus abolishing all immune effector functions. The hIgG4 Fab Arm Exchange Mutant sequence includes a mutation to suppress Fab Arm Exchange (S228P; EU numbering). An IL2 signal sequence (MYRMQLLSCIALSLALVTNS; SEQ ID NO: 9) can be located N-terminally of the variable region. It is used in expression vectors, which is cleaved during secretion and thus not in the mature antibody molecule. The mature protein (after secretion) starts with “EVQ” for the heavy chain and “DIM” for the light chain. Amino acid sequences of exemplary heavy chain constant regions are provided below:

hIgG1 Heavy Chain Constant Region
(SEQ ID NO: 10)
ASTKGPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVELFPPKPKDTLMISR
TPEVTCVVVDVSHEDPEVKENWYVDGVEVHNAKTKPREEQYNSTYRVVSVLTVLHQDWLNGKEYKCKVS
NKALPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK*
hIgG1 LALA Heavy Chain Constant Region
(SEQ ID NO: 12)
ASTKGPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTLMISR
TPEVTCVVVDVSHEDPEVKENWYVDGVEVHNAKTKPREEQYNSTYRVVSVLTVLHQDWLNGKEYKCKVS
NKALGAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK*
hIgG4 Heavy Chain Constant Region
(SEQ ID NO: 13)
ASTKGPSVFPLAPCSRSTSESTAALGCLVKDYFPEPVTVSWNSGALTSGVHTFPAVLQSSGLYSLSSVV
TVPSSSLGTKTYTCNVDHKPSNTKVDKRVESKYGPPCPSCPAPEFLGGPSVELFPPKPKDTLMISRTPE
VTCVVVDVSQEDPEVQFNWYVDGVEVHNAKTKPREEQFNSTYRVVSVLTVLHQDWLNGKEYKCKVSNKG
LPSSIEKTISKAKGQPREPQVYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSPGK*
hIgG4 Heavy Chain Constant Region
(SEQ ID NO: 20)
ASTKGPSVFPLAPCSRSTSESTAALGCLVKDYFPEPVTVSWNSGALTSGVHTFPAVLQSSGLYSLSSVV
TVPSSSLGTKTYTCNVDHKPSNTKVDKRVESKYGPPCPSCPAPEFLGGPSVELFPPKPKDTLMISRTPE
VTCVVVDVSQEDPEVQFNWYVDGVEVHNAKTKPREEQFNSTYRVVSVLTVLHQDWLNGKEYKCKVSNKG
LPSSIEKTISKAKGQPREPQVYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSLGK*
hIgG4 mut Heavy Chain Constant Region
(SEQ ID NO: 14)
ASTKGPSVFPLAPCSRSTSESTAALGCLVKDYFPEPVTVSWNSGALTSGVHTFPAVLQSSGLYSLSSVV
TVPSSSLGTKTYTCNVDHKPSNTKVDKRVESKYGPPCPPCPAPEFLGGPSVELFPPKPKDTLMISRTPE
VTCVVVDVSQEDPEVQFNWYVDGVEVHNAKTKPREEQFNSTYRVVSVLTVLHQDWLNGKEYKCKVSNKG
LPSSIEKTISKAKGQPREPQVYTLPPSQEEMTKNQVSLTCLVKGFYPSDIAVEWESNGQPENNYKTTPP
VLDSDGSFFLYSRLTVDKSRWQEGNVESCSVMHEALHNHYTQKSLSLSPGK*
hIgG4 mut Heavy Chain Constant Region
(SEQ ID NO: 21)
ASTKGPSVFPLAPCSRSTSESTAALGCLVKDYFPEPVTVSWNSGALTSGVHTFPAVLQSSGLYSLSSVV
TVPSSSLGTKTYTCNVDHKPSNTKVDKRVESKYGPPCPPCPAPEFLGGPSVELFPPKPKDTLMISRTPE
VTCVVVDVSQEDPEVQFNWYVDGVEVHNAKTKPREEQFNSTYRVVSVLTVLHQDWLNGKEYKCKVSNKG
LPSSIEKTISKAKGQPREPQVYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSLGK*

In some instances, the heavy chain constant region in an anti-Galectin-9 antibody disclosed herein (e.g., G9.2-17) may have the C-terminal Lysine (K) residue removed for, e.g., manufacturing purposes. The corresponding amino acid sequences of those having no terminal K residue are provided below:

hIgG1 Heavy Chain Constant Region with No C-Terminal Lysine
(SEQ ID NO: 24)
ASTKGPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLMISR
TPEVTCVVVDVSHEDPEVKENWYVDGVEVHNAKTKPREEQYNSTYRVVSVLTVLHQDWLNGKEYKCKVS
NKALPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPG*
hIgG1 LALA Heavy Chain Constant Region with No C-Terminal Lysine
(SEQ ID NO: 25)
ASTKGPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVELFPPKPKDTLMISR
TPEVTCVVVDVSHEDPEVKENWYVDGVEVHNAKTKPREEQYNSTYRVVSVLTVLHQDWLNGKEYKCKVS
NKALGAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPG*
hIgG4 Heavy Chain Constant Region with No C-Terminal Lysine
(SEQ ID NO: 26)
ASTKGPSVFPLAPCSRSTSESTAALGCLVKDYFPEPVTVSWNSGALTSGVHTFPAVLQSSGLYSLSSVV
TVPSSSLGTKTYTCNVDHKPSNTKVDKRVESKYGPPCPSCPAPEFLGGPSVELFPPKPKDTLMISRTPE
VTCVVVDVSQEDPEVQFNWYVDGVEVHNAKTKPREEQFNSTYRVVSVLTVLHQDWLNGKEYKCKVSNKG
LPSSIEKTISKAKGQPREPQVYTLPPSQEEMTKNQVSLTCLVKGFYPSDIAVEWESNGQPENNYKTTPP
VLDSDGSFFLYSRLTVDKSRWQEGNVESCSVMHEALHNHYTQKSLSLSPG*
hIgG4 Heavy Chain Constant Region with No C-Terminal Lysine
(SEQ ID NO: 27)
ASTKGPSVFPLAPCSRSTSESTAALGCLVKDYFPEPVTVSWNSGALTSGVHTFPAVLQSSGLYSLSSVV
TVPSSSLGTKTYTCNVDHKPSNTKVDKRVESKYGPPCPSCPAPEFLGGPSVELFPPKPKDTLMISRTPE
VTCVVVDVSQEDPEVQFNWYVDGVEVHNAKTKPREEQENSTYRVVSVLTVLHQDWLNGKEYKCKVSNKG
LPSSIEKTISKAKGQPREPQVYTLPPSQEEMTKNQVSLTCLVKGFYPSDIAVEWESNGQPENNYKTTPP
VLDSDGSFFLYSRLTVDKSRWQEGNVESCSVMHEALHNHYTQKSLSLSLG*
hIgG4 mut Heavy Chain Constant Region with No C-Terminal Lysine
(SEQ ID NO: 28)
ASTKGPSVFPLAPCSRSTSESTAALGCLVKDYFPEPVTVSWNSGALTSGVHTFPAVLQSSGLYSLSSVV
TVPSSSLGTKTYTCNVDHKPSNTKVDKRVESKYGPPCPPCPAPEFLGGPSVELFPPKPKDTLMISRTPE
VTCVVVDVSQEDPEVQFNWYVDGVEVHNAKTKPREEQFNSTYRVVSVLTVLHQDWLNGKEYKCKVSNKG
LPSSIEKTISKAKGQPREPQVYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSPG*
hIgG4 mut Heavy Chain Constant Region with No C-Terminal Lysine
(SEQ ID NO: 29)
ASTKGPSVFPLAPCSRSTSESTAALGCLVKDYFPEPVTVSWNSGALTSGVHTFPAVLQSSGLYSLSSVV
TVPSSSLGTKTYTCNVDHKPSNTKVDKRVESKYGPPCPPCPAPEFLGGPSVFLFPPKPKDTLMISRTPE
VTCVVVDVSQEDPEVQFNWYVDGVEVHNAKTKPREEQFNSTYRVVSVLTVLHQDWLNGKEYKCKVSNKG
LPSSIEKTISKAKGQPREPQVYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSLG*

In some embodiments, anti-Galectin-9 antibodies having any of the above heavy chain constant regions are paired with a light chain having the following light chain constant region:

Light Chain Constant Region
(SEQ ID NO: 11)
TVAAPSVEIFPPSDEQLKSGTASVVCLLNNFYPREAKVQWKVD
NALQSGNSQESVTEQDSKDSTYSLSSTLTLSKADYEKHKVYAC
EVTHQGLSSPVTKSENRGEC

Exemplary full length anti-Galectin-9 antibodies are provided below:

G9.2-17 hIgG1 Heavy Chain
(SEQ ID NO: 16)
EVQLVESGGGLVQPGGSLRLSCAASGFTVSSSSIHWVRQAPGKGLEWVAYISSSSGYTYYADSVKGRFT
ISADTSKNTAYLQMNSLRAEDTAVYYCARYWSYPSWWPYRGMDYWGQGTLVTVSSASTKGPSVFPLAPS
SKSTSGGTAALGCLVKDYFPEPVTVSWNSGALTSGVHTFPAVLQSSGLYSLSSVVTVPSSSLGTQTYIC
NVNHKPSNTKVDKKVEPKSCDKTHTCPPCPAPELLGGPSVELFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK*
G9.2-17 hIgG1 Heavy Chain with No C-terminal Lysine Residue
(SEQ ID NO: 30)
EVQLVESGGGLVQPGGSLRLSCAASGFTVSSSSIHWVRQAPGKGLEWVAYISSSSGYTYYADSVKGRFT
ISADTSKNTAYLQMNSLRAEDTAVYYCARYWSYPSWWPYRGMDYWGQGTLVTVSSASTKGPSVFPLAPS
SKSTSGGTAALGCLVKDYFPEPVTVSWNSGALTSGVHTFPAVLQSSGLYSLSSVVTVPSSSLGTQTYIC
NVNHKPSNTKVDKKVEPKSCDKTHTCPPCPAPELLGGPSVELFPPKPKDTLMISRTPEVTCVVVDVSHE
DPEVKENWYVDGVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPG*
G9.2-17 hIgG1 LALA Heavy Chain
(SEQ ID NO: 17)
EVQLVESGGGLVQPGGSLRLSCAASGFTVSSSSIHWVRQAPGKGLEWVAYISSSSGYTYYADSVKGRFT
ISADTSKNTAYLQMNSLRAEDTAVYYCARYWSYPSWWPYRGMDYWGQGTLVTVSSASTKGPSVFPLAPS
SKSTSGGTAALGCLVKDYFPEPVTVSWNSGALTSGVHTFPAVLQSSGLYSLSSVVTVPSSSLGTQTYIC
NVNHKPSNTKVDKKVEPKSCDKTHTCPPCPAPEAAGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALGAPIEKTISK
AKGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK*
G9.2-17 hIgG1 LALA Heavy Chain with No C-terminal Lysine Residue
(SEQ ID NO: 31)
EVQLVESGGGLVQPGGSLRLSCAASGFTVSSSSIHWVRQAPGKGLEWVAYISSSSGYTYYADSVKGRFT
ISADTSKNTAYLQMNSLRAEDTAVYYCARYWSYPSWWPYRGMDYWGQGTLVTVSSASTKGPSVFPLAPS
SKSTSGGTAALGCLVKDYFPEPVTVSWNSGALTSGVHTFPAVLQSSGLYSLSSVVTVPSSSLGTQTYIC
NVNHKPSNTKVDKKVEPKSCDKTHTCPPCPAPEAAGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALGAPIEKTISK
AKGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPG*
G9.2-17 hIgG4 Heavy Chain
(SEQ ID NO: 18)
EVQLVESGGGLVQPGGSLRLSCAASGFTVSSSSIHWVRQAPGKGLEWVAYISSSSGYTYYADSVKGRFT
ISADTSKNTAYLQMNSLRAEDTAVYYCARYWSYPSWWPYRGMDYWGQGTLVTVSSASTKGPSVFPLAPC
SRSTSESTAALGCLVKDYFPEPVTVSWNSGALTSGVHTFPAVLQSSGLYSLSSVVTVPSSSLGTKTYTC
NVDHKPSNTKVDKRVESKYGPPCPSCPAPEFLGGPSVFLFPPKPKDTLMISRTPEVTCVVVDVSQEDPE
VQFNWYVDGVEVHNAKTKPREEQFNSTYRVVSVLTVLHQDWLNGKEYKCKVSNKGLPSSIEKTISKAKG
QPREPQVYTLPPSQEEMTKNQVSLTCLVKGFYPSDIAVEWESNGQPENNYKTTPPVLDSDGSFFLYSRL
TVDKSRWQEGNVFSCSVMHEALHNHYTQKSLSLSPGK*
G9.2-17 hIgG4 Heavy Chain with No C-terminal Lysine Residue
(SEQ ID NO: 32)
EVQLVESGGGLVQPGGSLRLSCAASGFTVSSSSIHWVRQAPGKGLEWVAYISSSSGYTYYADSVKGRFT
ISADTSKNTAYLQMNSLRAEDTAVYYCARYWSYPSWWPYRGMDYWGQGTLVTVSSASTKGPSVEPLAPC
SRSTSESTAALGCLVKDYFPEPVTVSWNSGALTSGVHTFPAVLQSSGLYSLSSVVTVPSSSLGTKTYTC
NVDHKPSNTKVDKRVESKYGPPCPSCPAPEFLGGPSVFLFPPKPKDTLMISRTPEVTCVVVDVSQEDPE
VQFNWYVDGVEVHNAKTKPREEQFNSTYRVVSVLTVLHQDWLNGKEYKCKVSNKGLPSSIEKTISKAKG
QPREPQVYTLPPSQEEMTKNQVSLTCLVKGFYPSDIAVEWESNGQPENNYKTTPPVLDSDGSFFLYSRL
TVDKSRWQEGNVFSCSVMHEALHNHYTQKSLSLSPG*
G9.2-17 hIgG4 Heavy Chain
(SEQ ID NO: 22)
EVQLVESGGGLVQPGGSLRLSCAASGFTVSSSSIHWVRQAPGKGLEWVAYISSSSGYTYYADSVKGRFT
ISADTSKNTAYLQMNSLRAEDTAVYYCARYWSYPSWWPYRGMDYWGQGTLVTVSSASTKGPSVFPLAPC
SRSTSESTAALGCLVKDYFPEPVTVSWNSGALTSGVHTFPAVLQSSGLYSLSSVVTVPSSSLGTKTYTC
NVDHKPSNTKVDKRVESKYGPPCPSCPAPEFLGGPSVFLFPPKPKDTLMISRTPEVTCVVVDVSQEDPE
VQFNWYVDGVEVHNAKTKPREEQFNSTYRVVSVLTVLHQDWLNGKEYKCKVSNKGLPSSIEKTISKAKG
QPREPQVYTLPPSQEEMTKNQVSLTCLVKGFYPSDIAVEWESNGQPENNYKTTPPVLDSDGSFFLYSRL
TVDKSRWQEGNVFSCSVMHEALHNHYTQKSLSLSLGK*
G9.2-17 hIgG4 Heavy Chain with No C-terminal Lysine Residue
(SEQ ID NO: 33)
EVQLVESGGGLVQPGGSLRLSCAASGFTVSSSSIHWVRQAPGKGLEWVAYISSSSGYTYYADSVKGRFT
ISADTSKNTAYLQMNSLRAEDTAVYYCARYWSYPSWWPYRGMDYWGQGTLVTVSSASTKGPSVFPLAPC
SRSTSESTAALGCLVKDYFPEPVTVSWNSGALTSGVHTFPAVLQSSGLYSLSSVVTVPSSSLGTKTYTC
NVDHKPSNTKVDKRVESKYGPPCPSCPAPEFLGGPSVFLFPPKPKDTLMISRTPEVTCVVVDVSQEDPE
VQFNWYVDGVEVHNAKTKPREEQFNSTYRVVSVLTVLHQDWLNGKEYKCKVSNKGLPSSIEKTISKAKG
QPREPQVYTLPPSQEEMTKNQVSLTCLVKGFYPSDIAVEWESNGQPENNYKTTPPVLDSDGSFFLYSRL
TVDKSRWQEGNVFSCSVMHEALHNHYTQKSLSLSLG*
G9.2-17 hIgG4 Fab Arm Exchange mut Heavy Chain
(SEQ ID NO: 19)
EVQLVESGGGLVQPGGSLRLSCAASGFTVSSSSIHWVRQAPGKGLEWVAYISSSSGYTYYADSVKGRFT
ISADTSKNTAYLQMNSLRAEDTAVYYCARYWSYPSWWPYRGMDYWGQGTLVTVSSASTKGPSVFPLAPC
SRSTSESTAALGCLVKDYFPEPVTVSWNSGALTSGVHTFPAVLQSSGLYSLSSVVTVPSSSLGTKTYTC
NVDHKPSNTKVDKRVESKYGPPCPPCPAPEFLGGPSVFLFPPKPKDTLMISRTPEVTCVVVDVSQEDPE
VQFNWYVDGVEVHNAKTKPREEQFNSTYRVVSVLTVLHQDWLNGKEYKCKVSNKGLPSSIEKTISKAKG
QPREPQVYTLPPSQEEMTKNQVSLTCLVKGFYPSDIAVEWESNGQPENNYKTTPPVLDSDGSFFLYSRL
TVDKSRWQEGNVFSCSVMHEALHNHYTQKSLSLSPGK*
G9.2-17 hIgG4 Fab Arm Exchange mut Heavy Chain with No C-terminal Lysine Residue
(SEQ ID NO: 34)
EVQLVESGGGLVQPGGSLRLSCAASGFTVSSSSIHWVRQAPGKGLEWVAYISSSSGYTYYADSVKGRFT
ISADTSKNTAYLQMNSLRAEDTAVYYCARYWSYPSWWPYRGMDYWGQGTLVTVSSASTKGPSVFPLAPC
SRSTSESTAALGCLVKDYFPEPVTVSWNSGALTSGVHTFPAVLQSSGLYSLSSVVTVPSSSLGTKTYTC
NVDHKPSNTKVDKRVESKYGPPCPPCPAPEFLGGPSVFLFPPKPKDTLMISRTPEVTCVVVDVSQEDPE
VQFNWYVDGVEVHNAKTKPREEQFNSTYRVVSVLTVLHQDWLNGKEYKCKVSNKGLPSSIEKTISKAKG
QPREPQVYTLPPSQEEMTKNQVSLTCLVKGFYPSDIAVEWESNGQPENNYKTTPPVLDSDGSFFLYSRL
TVDKSRWQEGNVFSCSVMHEALHNHYTQKSLSLSPG*
G9.2-17 hIgG4 Fab Arm Exchange mut Heavy Chain
(SEQ ID NO: 23)
EVQLVESGGGLVQPGGSLRLSCAASGFTVSSSSIHWVRQAPGKGLEWVAYISSSSGYTYYADSVKGRFT
ISADTSKNTAYLQMNSLRAEDTAVYYCARYWSYPSWWPYRGMDYWGQGTLVTVSSASTKGPSVFPLAPC
SRSTSESTAALGCLVKDYFPEPVTVSWNSGALTSGVHTFPAVLQSSGLYSLSSVVTVPSSSLGTKTYTC
NVDHKPSNTKVDKRVESKYGPPCPPCPAPEFLGGPSVFLFPPKPKDTLMISRTPEVTCVVVDVSQEDPE
VQFNWYVDGVEVHNAKTKPREEQFNSTYRVVSVLTVLHQDWLNGKEYKCKVSNKGLPSSIEKTISKAKG
QPREPQVYTLPPSQEEMTKNQVSLTCLVKGFYPSDIAVEWESNGQPENNYKTTPPVLDSDGSFFLYSRL
TVDKSRWQEGNVFSCSVMHEALHNHYTQKSLSLSLGK*
G9.2-17 hIgG4 Fab Arm Exchange mut Heavy Chain with No C-terminal Lysine Residue
(SEQ ID NO: 35)
EVQLVESGGGLVQPGGSLRLSCAASGFTVSSSSIHWVRQAPGKGLEWVAYISSSSGYTYYADSVKGRFT
ISADTSKNTAYLQMNSLRAEDTAVYYCARYWSYPSWWPYRGMDYWGQGTLVTVSSASTKGPSVFPLAPC
SRSTSESTAALGCLVKDYFPEPVTVSWNSGALTSGVHTFPAVLQSSGLYSLSSVVTVPSSSLGTKTYTC
NVDHKPSNTKVDKRVESKYGPPCPPCPAPEFLGGPSVFLFPPKPKDTLMISRTPEVTCVVVDVSQEDPE
VQFNWYVDGVEVHNAKTKPREEQFNSTYRVVSVLTVLHQDWLNGKEYKCKVSNKGLPSSIEKTISKAKG
QPREPQVYTLPPSQEEMTKNQVSLTCLVKGFYPSDIAVEWESNGQPENNYKTTPPVLDSDGSFFLYSRL
TVDKSRWQEGNVFSCSVMHEALHNHYTQKSLSLSLG*

Any of the above heavy chain can be paired with a Light Chain of (SEQ ID NO: 15) shown below:

DIQMTQSPSSLSASVGDRVTITCRASQSVSSAVAWYQQKPGKAP
KLLIYSASSLYSGVPSRFSGSRSGTDFTLTISSLQPEDFATYYC
QQSSTDPITFGQGTKVEIKRTVAAPSVFIFPPSDEQLKSGTASV
VCLLNNFYPREAKVQWKVDNALQSGNSQESVTEQDSKDSTYSLS
STLTLSKADYEKHKVYACEVTHQGLSSPVTKSENRGEC*

In some embodiments, the anti-Galectin-9 antibody comprises a heavy chain IgG1 constant region that has at least 80% (e.g., 80%, 85%, 90%, 91%, 92%, 93%, 94%, 95%, 96%, 97%, 98%, or 99% and any increment therein) sequence identity to SEQ ID NO: 10. In one embodiment, the constant region of the anti-Galectin-9 antibody comprises a heavy chain IgG4 constant region comprising SEQ ID NO: 10. In one embodiment, the constant region of the anti-Galectin-9 antibody comprises a heavy chain IgG1 constant region consisting of SEQ ID NO: 10.

In some embodiments, the anti-Galectin-9 antibody comprises a heavy chain IgG4 constant region that has at least 80% (e.g., 80%, 85%, 90%, 91%, 92%, 93%, 94%, 95%, 96%, 97%, 98%, or 99% and any increment therein) sequence identity to SEQ ID NO: 20. In one embodiment, the constant region of the anti-Galectin-9 antibody comprises a heavy chain IgG4 constant region comprising SEQ ID NO: 20. In one embodiment, the constant region of the anti-Galectin-9 antibody comprises a heavy chain IgG4 constant region consisting of SEQ ID NO: 20.

In some embodiments, the constant region is from human IgG4. In one embodiment, the anti-Galectin-9 antibody comprises a heavy chain IgG4 constant region that has at least 80% (e.g., 80%, 85%, 90%, 91%, 92%, 93%, 94%, 95%, 96%, 97%, 98%, or 99% and any increment therein) sequence identity to SEQ ID NO: 13. In one embodiment, the anti-Galectin-9 antibody comprises a heavy chain IgG4 constant region comprising SEQ ID NO: 13. In one embodiment, the anti-Galectin-9 antibody comprises a heavy chain IgG4 constant region consisting of SEQ ID NO: 13.

In some embodiments, the constant region is from human IgG4. In one embodiment, the anti-Galectin-9 antibody comprises a heavy chain IgG4 constant region that has at least 80% (e.g., 80%, 85%, 90%, 91%, 92%, 93%, 94%, 95%, 96%, 97%, 98%, or 99% and any increment therein) sequence identity to SEQ ID NO: 20. In one embodiment, the anti-Galectin-9 antibody comprises a heavy chain IgG4 constant region comprising SEQ ID NO: 20. In one embodiment, the anti-Galectin-9 antibody comprises a heavy chain IgG4 constant region consisting of SEQ ID NO: 20.

In any of these embodiments, the anti-Galectin-9 antibody comprises a light chain constant region that has at least 80% (e.g., 80%, 85%, 90%, 91%, 92%, 93%, 94%, 95%, 96%, 97%, 98%, or 99% and any increment therein) sequence identity to SEQ ID NO: 11. In some embodiments, the anti-Galectin-9 antibody comprises a light chain constant region comprising SEQ ID NO: 11. In some embodiments, the anti-Galectin-9 antibody comprises a light chain constant region consisting of SEQ ID NO: 11.

In some embodiments, the IgG is a mutant with minimal Fc receptor engagement. In one example, the constant region is from a human IgG1 LALA. In one embodiment, the anti-Galectin-9 antibody comprises a heavy chain IgG1 constant region that has at least 80% (e.g., 80%, 85%, 90%, 91%, 92%, 93%, 94%, 95%, 96%, 97%, 98%, or 99% and any increment therein) sequence identity to SEQ ID NO: 12. In one embodiment, the anti-Galectin-9 antibody comprises a heavy chain IgG1 constant region comprising SEQ ID NO: 12. In one embodiment, the anti-Galectin-9 antibody comprises a heavy chain IgG1 constant region consisting of SEQ ID NO: 12.

In some embodiments, the anti-Galectin-9 antibody comprises a modified constant region. In some embodiments, the anti-Galectin-9 antibody comprise a modified constant region that is immunologically inert, e.g., does not trigger complement mediated lysis, or does not stimulate antibody-dependent cell mediated cytotoxicity (ADCC). ADCC activity can be assessed using methods disclosed in U.S. Pat. No. 5,500,362. In other embodiments, the constant region is modified as described in Eur. J. Immunol. (1999) 29:2613-2624; WO/1999/058572; and/or UK Patent Application No. 9809951.8. In some embodiments, the IgG4 constant region is a mutant with reduced heavy chain exchange. In some embodiments, the constant region is from a human IgG4 Fab Arm Exchange mutant S228P.

In one embodiment, the constant region of the anti-Galectin-9 antibody comprises a heavy chain IgG4 constant region that has at least 80% (e.g., 80%, 85%, 90%, 91%, 92%, 93%, 94%, 95%, 96%, 97%, 98%, or 99% and any increment therein) sequence identity to SEQ ID NO: 14. In one embodiment, the constant region of the anti-Galectin-9 antibody comprises a heavy chain IgG4 constant region comprising SEQ ID NO: 14. In one embodiment, the constant region of the anti-Galectin-9 antibody comprises a heavy chain IgG4 constant region consisting of SEQ ID NO: 14.

In one embodiment, the anti-Galectin-9 antibody comprises a heavy chain IgG4 constant region that has at least 80% (e.g., 80%, 85%, 90%, 91%, 92%, 93%, 94%, 95%, 96%, 97%, 98%, or 99% and any increment therein) sequence identity to SEQ ID NO: 21. In one embodiment, the anti-Galectin-9 antibody comprises a heavy chain IgG4 constant region comprising SEQ ID NO: 21. In one embodiment, the anti-Galectin-9 antibody comprises a heavy chain IgG4 constant region consisting of SEQ ID NO: 21.

In some embodiments, the anti-Galectin-9 antibody has chains corresponding to SEQ ID NO: 15 for the light chains; and the amino acid sequences of exemplary heavy chains correspond to SEQ ID NO: 10 (hIgG1); 12 (hIgG1 LALA); 13 (hIgG4); 20 (hIgG4); 14 (hIgG4 mut); and 21 (hIgG4 mut).

In some embodiments, the anti-Galectin-9 antibody has a light chain comprising, consisting essentially of, or consisting of SEQ ID NO: 15. In some embodiments, the anti-Galectin-9 antibody has a heavy chain comprising, consisting essentially of, or consisting of any one of the sequences selected from the group consisting of SEQ ID NO: 16-19, 22 and 23. In some embodiments, the anti-Galectin-9 antibody has a light chain comprising, consisting essentially of, or consisting of SEQ ID NO: 15 and a heavy chain comprising, consisting essentially of, or consisting of any one of the sequences selected from the group consisting of SEQ ID NO: 16-19. In some embodiments, the anti-Galectin-9 antibody has a light chain comprising SEQ ID NO: 15 and a heavy chain comprising any one of the sequences selected from the group consisting of SEQ ID NO: 16-19, 22 and 23. In some embodiments, the anti-Galectin-9 antibody has a light chain consisting essentially of SEQ ID NO: 15 and a heavy chain consisting essentially of any one of the sequences selected from the group consisting of SEQ ID NO: 16-19, 22 and 23. In some embodiments, the anti-Galectin-9 antibody has a light chain consisting of SEQ ID NO: 15 and a heavy chain consisting of any one of the sequences selected from the group consisting of SEQ ID NO: 16-19, 22 and 23. In one specific embodiment, the anti-Galectin-9 antibody has a light chain consisting essentially of SEQ ID NO: 15 and a heavy chain consisting essentially of SEQ ID NO: 19. In another specific embodiment, the anti-Galectin-9 antibody has a light chain consisting essentially of SEQ ID NO: 15 and a heavy chain consisting essentially of SEQ ID NO: 20.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 16. In one embodiment, the anti-Galectin-9 antibody comprises a heavy chain sequence comprising SEQ ID NO: 16. In one embodiment, the anti-Galectin-9 antibody comprises a heavy chain sequence consisting of SEQ ID NO: 16.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 17. In one embodiment, the anti-Galectin-9 antibody comprises a heavy chain sequence comprising SEQ ID NO: 17. In one embodiment, the anti-Galectin-9 antibody comprises a heavy chain sequence consisting of SEQ ID NO: 17.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 18. In one embodiment, the anti-Galectin-9 antibody comprises a heavy chain sequence comprising SEQ ID NO: 18. In one embodiment, the anti-Galectin-9 antibody comprises a heavy chain sequence consisting of SEQ ID NO: 18.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 22. In one embodiment, the anti-Galectin-9 antibody comprises a heavy chain sequence comprising SEQ ID NO: 22. In one embodiment, the anti-Galectin-9 antibody comprises a heavy chain sequence consisting of SEQ ID NO: 22.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 19. In one embodiment, the anti-Galectin-9 antibody comprises a heavy chain sequence comprising SEQ ID NO: 19. In one embodiment, the anti-Galectin-9 antibody comprises a heavy chain sequence consisting of SEQ ID NO: 19.

In one embodiment, the anti-Galectin-9 antibody comprises a heavy chain sequence having at least 80% (e.g., 80%, 85%, 90%, 91%, 92%, 93%, 94%, 95%, 96%, 97%, 98%, or 99% and any increment therein) sequence identity to SEQ ID NO: 23. In one embodiment, the anti-Galectin-9 antibody comprises a heavy chain sequence comprising SEQ ID NO: 23. In one embodiment, the anti-Galectin-9 antibody comprises a heavy chain sequence consisting of SEQ ID NO: 23.

In any of these embodiments, the anti-Galectin-9 antibody comprises a light chain sequence having at least 80% (e.g., 80%, 85%, 90%, 91%, 92%, 93%, 94%, 95%, 96%, 97%, 98%, or 99% and any increment therein) sequence identity to SEQ ID NO: 15. In some embodiments, the anti-Galectin-9 antibody comprises a light chain sequence comprising SEQ ID NO: 15. In some embodiments, the anti-Galectin-9 antibody comprises a light chain sequence consisting of SEQ ID NO: 15.

In specific examples, the anti-Galectin-9 antibody used in the treatment methods disclosed herein has a heavy chain of SEQ ID NO:19 and a light chain of SEQ ID NO:15. In some embodiments, the anti-Galectin-9 antibody used in the treatment methods disclosed herein is G9.2-17 IgG4. In some examples, such an anti-Galectin-9 antibody does not have the C-terminal lysine residue in its heavy chain.

### II. Preparation of Anti-Galectin-9 Antibodies

Antibodies capable of binding Galectin-9 as described herein can be made by any method known in the art, including but not limited to, recombinant technology. One example is provided below.

Nucleic acids encoding the heavy and light chain of an anti-Galectin-9 antibody as described herein can be cloned into one expression vector, each nucleotide sequence being in operable linkage to a suitable promoter. In one example, each of the nucleotide sequences encoding the heavy chain and light chain is in operable linkage to a distinct promoter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementarity ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters (M. Brown et al., Cell, 49:603-612 (1987); Gossen and Bujard (1992); M. Gossen et al., Natl. Acad. Sci. USA, 89:5547-5551 (1992)) combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(16):1392-1399 (2003)). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes both the heavy chain and the light chain of an anti-Galectin-9 antibody, as also described herein. The recombinant expression vector can be introduced into a suitable host cell (e.g., a dhfr− CHO cell) by a conventional method, e.g., calcium phosphate-mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

In one example, two recombinant expression vectors are provided, one encoding the heavy chain of the anti-Galectin-9 antibody and the other encoding the light chain of the anti-Galectin-9 antibody. Both of the two recombinant expression vectors can be introduced into a suitable host cell (e.g., dhfr− CHO cell) by a conventional method, e.g., calcium phosphate-mediated transfection. Alternatively, each of the expression vectors can be introduced into a suitable host cell. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of an anti-Galectin-9 antibody as described herein, vectors (e.g., expression vectors) containing such; and host cells comprising the vectors are within the scope of the present disclosure.

Anti-Galectin-9 antibodies thus prepared can be characterized using methods known in the art, whereby reduction, amelioration, or neutralization of Galectin-9 biological activity is detected and/or measured. For example, in some embodiments, an ELISA-type assay is suitable for qualitative or quantitative measurement of Galectin-9 inhibition of Dectin-1 or TIM-3 signaling.

The bioactivity of an anti-Galectin-9 antibody can verified by incubating a candidate antibody with Dectin-1 and Galectin-9, and monitoring any one or more of the following characteristics: (a) binding between Dectin-1 and Galectin-9 and inhibition of the signaling transduction mediated by the binding; (b) preventing, ameliorating, or treating any aspect of a hematologic malignancy; (c) blocking or decreasing Dectin-1 activation; (d) inhibiting (reducing) synthesis, production or release of Galectin-9. Alternatively, TIM-3 can be used to verify the bioactivity of an anti-Galectin-9 antibody using the protocol described above. Alternatively, CD206 can be used to verify the bioactivity of an anti-Galectin-9 antibody using the protocol described above.

In some embodiments, bioactivity or efficacy is assessed in a subject, e.g., by measuring peripheral and intra-tumoral T cell ratios, T cell activation, or by macrophage phenotyping.

Additional assays to determine bioactivity of an anti-Galectin-9 antibody include measurement of CD8+ and CD4+(conventional) T-cell activation (in an in vitro or in vivo assay, e.g., by measuring inflammatory cytokine levels, e.g., IFNgamma, TNFalpha, CD44, ICOS granzymeB, Perforin, IL2 (upregulation); CD26L and IL-10 (downregulation)); measurement of reprogramming of macrophages (in vitro or in vivo), e.g., from the M2 to the M1 phenotype (e.g., increased MHCII, reduced CD206, increased TNF-alpha and iNOS), Alternatively, levels of ADCC can be assessed, e.g., in an in vitro assay, as described herein.

### III. Methods of Treatment

The present disclosure provides methods for treating a hematologic malignancy, which are cancers that begin in blood-forming tissue, such as the bone marrow or in the cells of the immune system. Hematological malignancies include acute lymphoblastic leukemia, chronic lymphocytic leukemia, lymphomas, multiple myeloma, acute myelogenous leukemia, chronic myelogenous leukemia, myelodysplastic syndromes and the myeloproliferative neoplasms, such as essential thrombocythemia, polycythemia vera and myelofibrosis. A subject having a hematological malignancy can be identified by routine medical examination, e.g., laboratory tests, organ functional tests, CT scans, or ultrasounds.

### A. Pharmaceutical Compositions

Any of the anti-Gal9 antibodies disclosed herein (e.g., G9.2-17 (IgG4)), as well as the encoding nucleic acids or nucleic acid sets, vectors comprising such, can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™ PLURONICS™ or polyethylene glycol (PEG).

In some examples, the pharmaceutical composition described herein comprises liposomes containing the antibodies (or the encoding nucleic acids) which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The antibodies, or the encoding nucleic acid(s), may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

In some examples, the pharmaceutical composition is an injectable formulation, Injectable formulations may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous infusion, water soluble antibodies can be administered by the drip method, whereby a pharmaceutical formulation containing the antibody and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

### (B) Treatment of Hematological Malignancies

In some embodiments, the present disclosure provides a method for treating a hematologic malignancy, for example, a hematologic malignancy involving Gal9+ cancer cells using a combination of an effective amount of an anti-Galectin-9 antibody described herein, including but not limited to, G9.2-17 IgG4 (having a heavy chain of SEQ ID NO:19 and a light chain of SEQ ID NO:15), and an effective amount or effective amounts of one or more chemotherapeutics such as venetoclax and/or a hypomethylating agent (HMA) (e.g., azacytidine or decitabine).

### Exemplary Target Hematological Malignancies

In some examples, the method disclosed herein is applied to a human patient having a leukemia, for example, acute myelogenous leukemia (AML). In other examples, the method disclosed herein is applied to a human patient having a lymphoma, e.g., those disclosed herein. In yet another example, the method disclosed herein is applied to a human patient having myelodysplastic syndromes (MDS).

Acute myeloid leukemia (AML) is a blood cancer that starts in the blood and bone marrow and can progress rapidly. AML involves abnormal white blood cells produced by bone marrow, which fill the blood and bone marrow, leaving less space for healthy cells. Symptoms associated with AML include bleeding and anemia. Left untreated, AML can be life threatening.

Myelodysplastic syndromes (MDS) are conditions that can occur when the blood-forming cells in the bone marrow become abnormal. This leads to low numbers of one or more types of blood cells. In MDS, some of the cells in the bone marrow are abnormal (dysplastic) and have problems making new blood cells. Many of the blood cells formed by these bone marrow cells are defective. Defective cells often die earlier than normal cells, and the body also destroys some abnormal blood cells, leaving the patient without enough normal blood cells. Different cell types can be affected, although the most common finding in MDS is a shortage of red blood cells (anemia).

In some instances, the subject for treatment by the method disclosed herein is a human patient having high risk MDS. In some instances, the human patient may have very high risk MDS. See, e.g., Sekeres et al., “Prognosis of myelodysplastic neoplasms/syndromes (MDS) in adults” at uptodate.com/contents/prognosis-of-myelodysplastic-neoplasms-syndromes-mds-in-adults?topicRef=16121&source=see_link, the relevant disclosures of which are incorporated by reference for the subject matter and purpose referenced herein.

In some instances, the subject may be a human patient having a refractory disease, for example, a refractory AML, or a refractory MDS. As used herein, “refractory” refers to the tumor that does not respond to or becomes resistant to a treatment. In some instances, the subject may be a human patient having a relapsed disease, for example, a relapsed AML, or a relapsed MDS. As used herein, “relapsed” or “relapses” refers to the tumor that returns or progresses following a period of improvement (e.g., a partial or complete response) with treatment.

In some instances, the human patient for treatment by the method disclosed herein may be subject to frontline treatments such as conventional chemotherapy, immunotherapy, or surgery for treating the target disease as known in the art.

In some instances, the subject is a human patient having an elevated level of Galectin-9 as relative to a control level. The level of Galectin-9 can be a plasma or serum level of Galectin-9 in the human patient. In other examples, the level of Galectin-9 is the level of Galectin-9 of cancer cells within the tumor. In other examples, the level of Galectin-9 is the level of Galectin-9 of immune cells within the tumor. In other examples, the level of Galectin-9 can be the level of cell-surface Galectin-9, for example the level of Galectin-9 on cancer cells. In one example, the level of Galectin-9 can be the level of Galectin-9 expressed cancer cells, e.g., on the surface of cancer cells, or Galectin-9 expressed in immune cells. In some examples, the control level represents the level of Galectin-9 in healthy subjects. In some embodiments, the control level may be a baseline level prior to treatment.

Alternatively, or in addition, a human patient for the treatment provided herein may carry one or more molecular biomarkers such as one or more mutated genes, one or more genes with specific polymorphisms (e.g., single nucleotide polymorphisms or SNPs), or a combination thereof. Exemplary molecular biomarkers include, but are not limited to, ABL1, ASXL1 (e.g., C.1926_1927INSG (P.G646WFS*12) 15% VAF, BCOR (e.g., mutation at R342), BCORL1 (e.g., G1682RFS*4), BRAF (e.g., K601N mutant), CBFBMYH1, CBL (e.g., C.1096-1G>T 59.7% VAF (228X consensus coverage) or C.1096-1G>C 18.6% VAF (113X consensus coverage)), CBLB (AF=53.1% MF3+), CEBPA, CSF3R, CUX1, DDX41 (e.g., C.1574G>A (P.R525H) 26.9% VAF), DNMT3A (e.g., C.2478+1G>A 32.7% VAF, or at W305), EZH2, GATA2, IDH2 (e.g., mutation), JAK2, KMT1A (e.g., rearranged), KRAS (e.g., T58I mutation), MPL, MUTYH (e.g., G396D/splice region variant −LOF, NF1 (e.g., C.3198-3_3200DELINES(7) splice region variant −LOF, NPM1 (e.g., mutation), NRAS (e.g., mutation), NSD1, p53 (e.g., TP53stat mutation), PIHF6 (e.g., C.971+1G>C 33% VAF), PMLRARA, PD_1, RAD21 (e.g., splice site C.1621-2A>G mutation), RUNX1 (e.g., C.601C>T (P.R201*) 18.4% VAF), RUNX1T1; SETBP1 (e.g., G870S/missense variant-GOF and/or AF=9.1%), SF3B1, SRFS2, SRSF2 (e.g., mutation), STAG2 (e.g., E403SFS*8), TBL1XR1/MECOM fusion, TET2 (e.g., mutation), U2AF1 (e.g., mutation of S34F), WT1, and/or ZRSR2.

To identify such a subject, a suitable biological sample can be obtained from a subject who is suspected of having the hematologic malignancy and the biological sample can be analyzed to determine the level of Galectin-9 contained therein (e.g., free, cell-surface expressed, or total) using conventional methods, e.g., ELISA or FACS. In some embodiments, organoid cultures are prepared, e.g., as described herein, and used to assess Galectin-9 levels in a subject. Single cells derived from certain fractions obtained as part of the organoid preparation process are also suitable for assessment of Galectin-9 levels in a subject. In some instances, an assay for measuring the level of Galectin-9, either in free form or expressed on cell surface, involves the use of an antibody that specifically binds the Galectin-9 (e.g., specifically binds human Galectin-9). Any of the anti-Galectin-9 antibodies known in the art can be tested for suitability in any of the assays described above and then used in such assays in a routine manner. In some embodiments, an antibody described herein (e.g., a G9.2-17 antibody) can be used in such as assay. In some embodiments, an antibody described in U.S. Pat. No. 10,344,091 and WO2019/084553, the relevant disclosures of each of which are incorporated by reference for the purpose and subject matter referenced herein. In some examples, the anti-Galectin-9 antibody is a Fab molecule. Assay methods for determining Galectin-9 levels as disclosed herein are also within the scope of the present disclosure.

In some embodiments, a human patient to be treated by any of the methods disclosed herein may meet one or more of the inclusion and exclusion criteria listed in Example 1 below. For example, such a human patient may meet all of the inclusion and exclusion criteria listed in Example 1 below.

### Exemplary Treatment Conditions

In some embodiments, an effective amount of the anti-Gal9 antibody disclosed herein (e.g., G9.2-17 IgG4) in combination with one or more chemotherapeutics such as a Bcl2 inhibitor (e.g., venetoclax) and/or a hypomethylating agent (HMA) (e.g., azacytidine or decitabine) may be given to a subject (e.g., a human patient, who can be an adult or a child) in need of the treatment, e.g., at a suitable dosage and a suitable dosing frequency. In some embodiments, the antibody may be administered to the subject via a suitable route (e.g., intravenous infusion) at a dose of about 2 mg/kg to about 16 mg/kg once every week. Alternatively, the antibody may be administered to the subject via the suitable route at a dose of about 2 mg/kg to about 16 mg/kg once every two weeks. See also disclosures herein.

In some embodiments, the anti-Gal9 antibody (e.g., G9.2-17 IgG4), in combination with one or more of the chemotherapeutics disclosed herein, may be administered to the subject at a dose of about 2 mg/kg to about 16 mg/kg once every week by intravenous infusion. In one example, the anti-Gal9 antibody disclosed herein (e.g., G9.2-17 (IgG4)) may be administered to the subject at a dose of about 2 mg/kg to about 4 mg/kg once every week by intravenous infusion (e.g., about 2 mg/kg, about 3 mg/kg, about 4 mg/kg, or any increment therein). In another example, the anti-Gal9 antibody disclosed herein (e.g., G9.2-17 (IgG4)) may be administered to the subject at a dose of about 4 mg/kg to 7.5 mg/kg once every week by intravenous infusion (e.g., about 4 mg/kg, about 5 mg/kg, about 6 mg/kg, about 7.5 mg/kg, or any increment therein). In still another example, the anti-Gal9 antibody disclosed herein (e.g., G9.2-17 (IgG4)) may be administered to the subject at a dose of about 7.5 mg/kg to 12 mg/kg once every week by intravenous infusion (e.g., about 7.5 mg/kg, about 8 mg/kg, about 9 mg/kg, about 10 mg/kg, about 11 mg/kg, about 12 mg/kg, or any increment therein). In yet another example, the anti-Gal9 antibody disclosed herein (e.g., G9.2-17 (IgG4)) may be administered to the subject at a dose of about 12 mg/kg to about 16 mg/kg once every week by intravenous infusion (e.g., about 12 mg/kg, about 13 mg/kg, about 14 mg/kg, about 15 mg/kg, about 16 mg/kg, or any increment therein). In some specific examples, the anti-Gal9 antibody may be administered to the subject at a dose of about 2 mg/kg, about 4 mg/kg, about 7.5 mg/kg, about 12 mg/kg, or about 16 mg/kg once every week by intravenous infusion.

Alternatively, the anti-Gal9 antibody (e.g., G9.2-17 IgG4), in combination with one or more of the chemotherapeutics disclosed herein, may be administered to the subject at the same doses disclosed above once every two weeks by intravenous infusion.

The term “about” or “approximately” means within an acceptable error range for the particular value as determined by one of ordinary skill in the art, which are dependent in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

An effective amount of the pharmaceutical composition described herein can be administered to a subject (e.g., a human) in need of the treatment via a suitable route, systemically or locally. In some embodiments, the anti-Galectin-9 antibodies are administered by intravenous administration, e.g., as a bolus or by continuous infusion over a period of time, by intramuscular, intraperitoneal, intracerebrospinal, subcutaneous, intra-arterial, intra-articular, intrasynovial, intrathecal, intratumoral, sub-urothelial, oral, inhalation or topical routes. In one embodiment, the anti-Galectin-9 antibody is administered to the subject by intravenous infusion. In one embodiment, the anti-galectin-9 antibody is administered to the subject intraperitoneally.

As used herein, “an effective amount” refers to the amount of each active agent required to confer therapeutic effect on the subject, either alone or in combination with one or more other active agents. In some embodiments, the therapeutic effect is reduced Galectin-9 activity and/or amount/expression, reduced Dectin-1 signaling, reduced TIM-3 signaling, reduced CD206 signaling, or increased anti-tumor immune responses in the tumor microenvironment. Non-limiting examples of increased anti-tumor responses include increased activation levels of effector T cells or switching of the TAMs from the M2 to the M1 phenotype. In some cases, the anti-tumor response includes increased ADCC responses. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally contribute to the determination of the dosage. For example, antibodies that are compatible with the human immune system, such as humanized antibodies or fully human antibodies, are in some instances used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, a symptom of the disease or disorder, or the predisposition toward the disease or disorder.

Alleviating a target disease/disorder includes delaying the development or progression of the disease or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

In one example, dosages for an antibody as described herein are determined empirically in individuals who have been given one or more administration(s) of the antibody. Individuals are given incremental dosages of the antagonist. To assess efficacy of the antagonist, an indicator of the disease/disorder can be followed.

In some examples, the human patient who has AML is administered the anti-Gal9 antibody, venetoclax (e.g., VENCLEXTA®), and a hypomethylating agent (HMA), which is azacytidine (e.g., Vidaza®) or decitabine (e.g., DACOGEN®).

In some specific examples, the treatment regimen comprises one or more of a cycle of 28 days, wherein in each cycle: the anti-Gal9 antibody is administered to the human patient on Day 1, Day 8, Day 15, and Day 22 at a dose of about 4 mg/kg, about 7.5 mg/kg, about 12 mg/kg, or about 16 mg/kg once every week by intravenous infusion; the venetoclax is administered to the human patient orally at 100 mg on Day 1, at 200 mg on Day 2, and at 400 mg on each day of Days 3-28; and the azacytidine at 75 mg/m² subcutaneously or intravenously on each of Days 1-7.

In other specific examples, the treatment regimen comprises one or more of a cycle of 28 days, wherein in each cycle: the anti-Gal9 antibody is administered to the human patient on Day 1, Day 8, Day 15, and Day 22 at a dose of about 4 mg/kg, about 7.5 mg/kg, about 12 mg/kg, or about 16 mg/kg once every week by intravenous infusion; the venetoclax is administered to the human patient orally at 100 mg on Day 1, at 200 mg on Day 2, and at 400 mg on each of Days 3-28; and the decitabine at 20 mg/m2 intravenously on each day of Days 1-5. For this regimen, the administration of the decitabine is repeated every 28 days.

In any of the methods of treating AML described herein, venetoclax is administered at a reduced dose or is terminated when severe venetoclax-associated toxicity occurs.

In some examples, the human patient who has MDS is administered the anti-Gal9 antibody and a hypomethylating agent (HMA), which is azacytidine or decitabine.

In some specific examples, the treatment regimen comprises one or more of a cycle of 28 days, wherein in each cycle: the anti-Gal9 antibody is administered to the human patient on Day 1, Day 8, Day 15, and Day 22 at a dose of about 4 mg/kg, about 7.5 mg/kg, about 12 mg/kg, or about 16 mg/kg once every week by intravenous infusion; and the azacytidine at 75 mg/m² subcutaneously or intravenously on each of Days 1-7.

In other specific examples, the treatment regimen comprises one or more of a cycle of 28 days, wherein in each cycle: the anti-Gal9 antibody is administered to the human patient on Day 1, Day 8, Day 15, and Day 22 at a dose of about 4 mg/kg, about 7.5 mg/kg, about 12 mg/kg, or about 16 mg/kg once every week by intravenous infusion; and the decitabine at 20 mg/m2 intravenously on each day of Days 1-5. For this regimen, the administration of the decitabine is repeated every 28 days.

In any of the methods of treating MDS described herein, azacytidine or decitabine is administered at a reduced dose or terminated when drug-related toxicity occurs.

### Monitoring Treatment Responses

Treatment efficacy for a target hematologic malignancy as disclosed herein can be assessed by methods well-known in the art.

In some embodiments, anti-tumor activity of any of treatment methods disclosed herein may be monitored by conventional approaches. For example, before, during, and after the administration of the antibody and optionally the second therapeutic agent, cancerous cells and/or biomarkers in a subject can be measured in a biological sample, such as blood, serum, plasma, urine, peritoneal fluid, and/or a biopsy from a tissue or organ. Results thus obtained can be analyzed to assess treatment efficacy. For examples, the levels of Gal9+ cancer cells may be measured before, during, and after the treatment to assess the direct killing effect of the anti-Gal9 antibody such as G9.2-17(IgG4).

Alternatively or in addition, response to treatment can also be characterized by one or more of immunophenotype (e.g., to assess immune modulating effects) in blood and tumors, cytokine profile (serum), soluble galectin-9 levels in blood (serum or plasma), galectin-9 tumor tissue expression levels and pattern of expression by immunohistochemistry (tumor, stroma, immune cells), tumor mutational burden (TMB), mismatch repair status, or tumor markers relevant for the disease (e.g., as measured at approximately 3 months, 6 months or 12 months, or at a later time or at any other clinically indicated time point).

In some examples, changes in levels of immune cells and immune cell markers in the blood or in tumors, e.g., immune activation, may be determined before and after the treatment. Such changes can be measured in patient blood and tissue samples using methods known in the art, such as multiplex flow cytometry and multiplex immunohistochemistry. For example, a panel of phenotypic and functional PBMC immune markers can be assessed at baseline prior to commencement of the treatment and at various time point during treatment. Table 2 lists non-limiting examples of markers useful for these assessment methods. Flow cytometry (FC) is a fast and highly informative method of choice technology to analyze cellular phenotype and function and has gained prominence in immune phenotype monitoring. It allows for the characterization of many subsets of cells, including rare subsets, in a complex mixture such as blood, and represents a rapid method to obtain large amounts of data. Advantages of FC are high speed, sensitivity, and specificity. Standardized antibody panels and procedures can be used to analyze and classify immune cell subtypes. Multiplex IHC is a powerful investigative tool, which provides objective quantitative data describing the tumor immune context in both immune subset number and location and allows for multiple markers to be assessed on a single tissue section. Computer algorithms can be used to quantify IHC-based biomarker content from whole slide images of patient biopsies, combining chromogenic IHC methods and stains with digital pathology approaches.

TABLE 2
PBMC phenotyping markers

| | PBMC phenotyping | | PBMC phenotyping |
|---|---|---|---|
| | markers | | markers |
| CD3 | Total T cells | CD16 | NK cells |
| CD4 | CD4+ T cells | CD11b | Monocytes/macrophages |
| CD8 | CD8+ T cells | CD11c | Monocytes/macrophages, |
| | | | DCs |
| CD25 | Treg activation | CD14 | Monocyte subsets, |
| | | | macrophages |
| CD27 | T cell maturation; | CD33 | Total myeloid cells |
| | B cell | | |
| | naïve/memory | | |
| CD38 | T cell maturation; | FceR1 a | Antigen presenting DC |
| | B cell | | cells |
| | naïve/memory | | |
| CD45RA | Naïve/memory cells | CD19 | Total B cells |
| CD45RO | Naïve/memory cells | T-bet | T cells subsets |
| CD56 | NKT/NK cells | gdTCR | Gamma delta T cells |
| | (T cell subset) | | |
| CD127 | T cell subsets | CD274 | Checkpoint |
| | | (PDL-1) | |
| CD152 | Checkpoint | Tim-3 | Checkpoint |
| (CLTA-4) | | | |
| CD279 | Checkpoint | TCRVa24- | iNKT cells |
| (PD-1) | | Ja18 | |
| FoxP3 | Treg cells | Live/dead | General |
| HLA-DR | Activation/Antigen | CD45 | General |
| | presentation | | |

A subject being treated by any of the anti-Gal9 antibodies disclosed herein (e.g., G9.2-17(IgG4)) in combination with a second therapeutic agent (e.g., a chemotherapeutic agent) as disclosed herein, may be monitored for occurrence of adverse effects (for example, severe adverse effects). Exemplary adverse effects to monitor are provided in Example 1 below. If occurrence of adverse effects is observed, treatment conditions may be changed for that subject. For example, the dose of the anti-galectin-9 antibody may be reduced and/or the dosing interval may be extended. Suitability and extent of reduction may be assessed by a qualified clinician. In some embodiments, one or more dose reductions by about 10% to about 80% of a previous dose level can be implemented. In some embodiments, one or more dose reductions by about 10% to about 20%, about 20% to about 30%, about 30% to about 40%, about 40% to about 50%, about 50% to about 60%, or about 70% to about 80% of a previous dose level can be implemented. In some embodiments, one or more dose reductions by 10% to 20%, 20% to 30%, 30% to 40%, 40% to 50%, 50% to 60%, or 70% to 80% of a previous dose level can be implemented. In some embodiments, one or more dose reductions by about 10%, by about 20%, by about 30%, by about 40%, by about 50%, by about 60%, by about 70%, or by about 80% of a previous dose level can be implemented. In some embodiments, one or more dose reductions by 10%, by 20%, by 30%, by 40%, by 50%, by 60%, by 70%, or by 80% of a previous dose level are implemented. Alternatively, or in addition, the dose of the second therapeutic agent can be reduced and/or the dosing interval of the checkpoint inhibitor may be extended. In some instances (e.g., occurring of life-threatening adverse effects), the treatment may be terminated.

Any of the methods disclosed herein may involve reducing the dose of the anti-Gal9 antibody and/or the chemotherapeutic agent(s) when one or more adverse events (e.g., those disclosed herein) are observed during the treatment. For any of the treatment methods described herein, the subject is free of a treatment comprising an immunosuppressant CYP3A4 substrate, which optionally is tacrolimus, cyclosporine, everolimus, or sirolimus.

### IV. Kits for Use in Treatment of Diseases Associated with Galectin-9

The present disclosure also provides kits for use in treating or alleviating a hematologic malignancy such as those disclosed herein (e.g., AML or MDS). Such kits can include one or more containers comprising an anti-Gal9 antibody, e.g., any of those described herein (e.g., G9.2-17(IgG4), and a second therapeutic agent (e.g., a chemotherapeutic agent as disclosed herein) to be co-used with the anti-Gal9 antibody, which is also described herein.

In some embodiments, the kit can comprise instructions for use in accordance with any of the methods described herein. The included instructions can comprise a description of administration of the anti-Gal9 antibody, and the second therapeutic agent, to treat, delay the onset, or alleviate a target disease as those described herein. In some embodiments, the kit further comprises a description of selecting an individual suitable for treatment based on identifying whether that individual has the target disease, e.g., applying the diagnostic method as described herein. In still other embodiments, the instructions comprise a description of administering an antibody to an individual at risk of the target disease.

The instructions relating to the use of an anti-Gal9 antibody generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating the target hematologic malignancy. In some embodiments, instructions are provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. In some embodiments, a kit has a sterile access port (for example the container may be an intravenous solution bag or a vial having a stopper pierceable by a hypodermic injection needle). In some embodiments, the container also has a sterile access port (for example the container is an intravenous solution bag or a vial having a stopper pierceable by a hypodermic injection needle). At least one active agent in the composition is an anti-Gal9 antibody as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present invention employs, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: A Phase 1 Open-Label, Multi-Center Study of the Safety, Pharmacokinetics (PK), and Anti-Tumor Activity of G9.2-17(IgG4) in Patients with Relapsed/Refractory Acute Myeloid Leukemia (AML), or with Relapsed/Refractory, High-Risk Myelodysplastic Syndrome (MDS)

G9.2-17(IgG4) is a fully human immunoglobulin gamma (IgG)4 monoclonal antibody (mAb) targeting the carbohydrate recognition domain 2 (CRD2) of Galectin-9 (gal-9) protein and is being developed for the treatment of patients with relapsed and refractory solid tumors and hematologic malignancies. Gal-9 plays a critical regulatory role in the anti-tumor immune responses. Gal-9, which is overexpressed and/or secreted in many cancer types (solid tumors and hematologic malignancies), functions as an immunosuppressor, conferring immune privilege to tumor cells and disabling immune-mediated cancer attack by regulating macrophages, T cells, natural killer (NK) cells, and myeloid-derived suppressor cells, as well as hindering cancer cell susceptibility to cytotoxic T cell induced death. G9.2-17(IgG4) has been designed to induce blockade of gal-9 and to interfere with the immunosuppressive functions of gal-9, which can result in immune reactivation and inhibition/control of tumor growth.

While conventional chemotherapy, with or without stem cell transplant, is still pivotal in curative treatment and/or disease control for AML because of its high proliferation intensity, molecular targeted approaches remain a practical addition and/or alternative, especially for patients with relapsed/refractory disease or for AML patients unable to tolerate intensive treatment. Advances and clinical applications of successful IO therapy for AML are awaited.

### Summary of Nonclinical Studies

High gal-9 expression in human tumor tissue correlates with worse disease outcomes. In vitro, G9.2-17(IgG4) binds the CRD2 domain of gal-9 with low nanomolar/picomolar affinity, and binds gal-9 expressed on the cell surface of CRL-2134 human colon cancer cells with an affinity of 0.54 nM. In vitro, G9.2-17(IgG4) inhibited the secretion of TGF-β and IL-10 from human M2 immunosuppressive macrophages, and reactivated T-cells in PDOTs. In addition, G9.2-17(IgG4) induced apoptosis of AML cells, on which gal-9 surface expression is significantly increased (>100-fold) compared to healthy blood cells.

In peripheral blood mononuclear cells (PBMC) derived from healthy donors and donors with non-hematologic solid tumor malignancies, G9.2-17(IgG4) does not induce cytokine release; G9.2-17(IgG4) also did not induce antibody-dependent cellular cytotoxicity (ADCC) and had moderate antibody-dependent cellular phagocytosis (ADCP) activity in vitro.

In vivo, G9.2-17(IgG4) exhibited anti-tumor activity in murine models of PDAC (pancreatic ductal adenocarcinoma), melanoma, and colorectal cancer (CRC), and, as both a single agent and in combination with chemotherapy, conferred a survival benefit in a murine AML xenograft model.

In a Good Laboratory Practice (GLP)-compliant, repeat-dose toxicity study in rats, G9.2-17(IgG4) administered intravenously (IV) at 100 and 300 mg/kg/day on Days 1, 8, 15, 22, and 29 was well-tolerated. There were no G9.2-17(IgG4)-related changes in evaluated parameters and as such, the no observed adverse effect level (NOAEL) was considered to be 300 mg/kg/day. A follow-up GLP-compliant study to evaluate potential toxicity of G9.2-17(IgG4) administered by IV injection to Sprague Dawley Rats once weekly for 13 consecutive weeks also found no G9.2-17(IgG4)-related changes in evaluated parameters and confirmed the NOAEL of 300 mg/kg/day.

In a GLP-compliant IV infusion toxicity study in cynomolgus monkeys, the toxicity and toxicokinetics (TK) of G9.2-17(IgG4) following once weekly 30-minute IV infusions for 5 consecutive doses at 100 and 300 mg/kg was assessed. These doses were well-tolerated with no adverse findings. The high dose was selected as the maximal feasible dose based on test article formulation concentrations and maximal volume allowed by IV infusion over 30 minutes. Therefore, under the conditions of this study the NOEL was 300 mg/kg, the highest dose level evaluated.

### Summary of Clinical Studies

Preliminary data from an ongoing clinical study with G9.2-17(IgG4) indicate that, following a single 60-minute IV infusion in Cycle 1, serum G9.2-17(IgG4) Cₘₐₓ and AUC₀₋₃₃₆ₕ increased approximately dose proportionally. Median Cmax values after dose 1 ranged from 4.09 μg/mL in the 0.2 mg/kg dose group to 289 μg/mL in the 16 mg/kg dose group, with Tₘₐₓ observed at or shortly after the end of infusion. Across the dose cohorts, the median CL values after the first dose ranged from 0.251 mL/hr/kg to 0.633 mL/hr/kg and the median V_{z} ranged between 41.8 and 83.0 mL/kg, which is consistent with blood volume (~65 to 75 mL/kg), indicating limited extravascular distribution. Median T_{1/2} values ranged from 57.9 to 115 hours (2.41 to 4.79 days) after the first dose across all dose groups; no dose-dependent differences in T_{1/2} were observed. For patients with Cycle 3 PK parameters only minimal accumulation was observed; the accumulation ratios comparing dose 1 (Cycle 1) exposure to dose 5 (Cycle 3) exposure for AUC₀₋₃₃₆ₕ and Cₘₐₓ were less than 1.5.

Preliminary safety results from Study G9.2-17(IgG4)-2020-01 showed no apparent trends in adverse event (AE) frequency between dosing groups, and no dose-limiting toxicities (DLTs) were observed. Of the 17 total patients, 7 patients (41.2%) had 11 serious adverse events (SAEs), none of which were assessed as related to G9.2-17(IgG4). The only SAE term reported for more than 1 patient was pleural effusion, which was reported for 1 patient each in the 10 mg/kg IV every week (QW) and 16 mg/kg IV Q2W groups. One patient in the 16 mg/kg IV Q2W group had a Grade 5 SAE of intestinal perforation that was unrelated to G9.2-17(IgG4). Overall, no DLTs and no G9.2-17(IgG4) and no G9.2-17(IgG4)-related SAEs were observed.

No immune-mediated adverse reactions (IMARs) nor any cytokine release/tumor lysis-type side effects have been observed at dose levels ranging from 0.2 to 16 mg/kg Q2W and at 10 mg/kg IV QW.

G9.2-17(IgG4) has signals of single agent clinical benefit with dose escalation continuing. See the results summarized in Table 10 below.

TABLE 10
G9.2-17(IgG4) Dose Escalation Results

| Cohorts | Patient Number | Results |
|---|---|---|
| Cohort 1: 2 mg/kg | 5 | 2 stable disease (SD) |
| | | 3 progressive disease (PD) |
| Cohort 2: 4 mg/kg | 4 | 2 stable disease (SD) |
| | | 2 progressive disease (PD) |
| Cohort 3: 7.5 mg/kg | 4 | 4 stable disease (SD) |
| Cohort 4: 12 mg/kg | 4 | Response assessment pending |

Further, four heavily pretreated AML patients have been successfully treated with G9.2-17(IgG4) at 7.5 mg/kg for median of 80 days. All four patients achieved stable disease and three patients had blast reduction. One patient has seen clearance of blasts from blood and two patients have achieved significant blast reduction in bone marrow. The results of the clinical data is provided in Table 11 below.

TABLE 11
Treatment of Heavily Pretreated AML Patients by G9.2-17(IgG4) at 7.5 mg/kg

| Number of | | Disease Best | | |
|---|---|---|---|---|
| Prior Lines | Treatment | Response per | Blasts | Blasts |
| of Therapy | Duration | ELN guidelines | at Baseline (%) | Restaging (%) |
| 3 | 91 days | Stable Disease | Peripheral: 9.8% | Peripheral: 1.4% at C3 |
| 3 | 68 days | Stable Disease | BM: 50-60% | BM: 20-30% at C2 |
| 4 | 147 days | Stable Disease | BM: 15-20% | BM: 5-10% at C3 |
| 4 | 50 days | Stable Disease | BM: 23% | BM: 44% at C2 |

In a study of single agent venetoclax in relapse/refractory AML, the median time on study was about two months (60 days). In this case, the median treatment with G9.2-17(IgG4) for 80 days is a favorable result.

### Expected Benefits of G9.2-17(IgG4) in AML and MDS

Expected benefits of G9.2-17(IgG4) in the AML/MDS relapsed/refractory disease setting are as follows:
Favorable safety profile based on the observations from the solid tumor study, as well as the 3-month rat toxicity study and the 4-week non-human primate repeat-dose study.
Potential immunomodulation peripherally and within the bone marrow are monitored by PD and efficacy assessments.
Potential direct anti-leukemic effect.
Potential clinical improvement of patients.

### I. Study Objectives

### Primary Objectives

The primary objectives are:
To establish the safety and tolerability of G9.2-17(IgG4) as a single agent and in combination with venetoclax and/or an HMA
To recommend the dose or doses of G9.2-17(IgG4) for Phase 2 (RP2D) in Part 1 and Part 2
To determine the incidence of DLTs during Cycle 1 (28 days) across dose levels in Part 1 and Part 2

### Secondary Objectives

The secondary objectives are:
To determine the preliminary efficacy of G9.2-17(IgG4) as a single treatment and in combination with venetoclax and/or a HMA
To characterize the PK profile of G9.2-17(IgG4) in Part 1 and Part 2

### Exploratory Objectives

The exploratory objectives are:
To study PD markers, including immunological and molecular changes in the peripheral blood and bone marrow in Part 1 and Part 2
To assess the immunogenicity of G9.2-17(IgG4) in Part 1 and Part 2

### II. Study Endpoints

### Primary Endpoints in Part 1 and Part 2

The primary endpoints are:
Evaluation of safety parameters including AEs, vital sign measurements, clinical safety laboratory tests, 12-lead electrocardiogram (ECG), echocardiography/cardiac ultrasound (ECHO), physical examinations
Determination of the biologically effective dose for G9.2-17(IgG4)
Determination of the maximum tolerated dose (MTD) for G9.2-17(IgG4)
Determination of the RP2D dose or doses for G9.2-17(IgG4)
Evaluation of DLTs during Cycle 1

### Secondary Endpoints in Part 1 and Part 2

The secondary endpoints to determine preliminary efficacy (disease response) are shown in Tables 3 and 4 below.

TABLE 3
AML Efficacy Endpoints and Definitions in Part 1 and Part 2

| | |
|---|---|
| Efficacy Endpoint | Definition |

Natural History

| | |
|---|---|
| Rate of composite complete remission | CR: |
| (CR + CR with incomplete | Bone marrow blasts ≤5% |
| hematological recovery [CRi]) | Absence of circulating blasts with Auer rods |
| | Absence of extramedullary disease |
| | Platelets ≥100 × 10⁹/L |
| | ANC ≥1 × 10⁹/L |
| | Red blood cell (RBC) transfusion independent |
| | CRi |
| | All CR criteria and transfusion independence but |
| | with persistence of neutropenia, i.e., absolute |
| | neutrophil count of <1 × 10⁹/L and/or platelet |
| | count <100 × 10⁹/L |
| Rate of Molecular Residual Disease | MRD: |
| (MRD) clearance among patients with: | RT-qPCR (genetic lesions) |
| CR, CRi, CR + CRi, Morphologic | MFC (immunophenotype markers) |
| Leukemia-free State (MLFS) | MLFS: |
| | Bone marrow blasts ≤5% |
| | Absence of blasts with Auer rods |
| | Absence of extramedullary disease |
| | No hematologic recovery required |
| | CR: defined above |
| | CRi: defined above |
| Rate of CR | CR: defined above |
| Overall Remission Rate (ORR = CR + | See above for definitions of CR, CRi, MLFS |
| CRi + Rate of Partial | PR: |
| Remission (PR) + MLFS) | All hematologic criteria of CR |
| | Decrease of pretreatment bone marrow blast |
| | percentage by at least half to a value 5% to 25% (if |
| | the pretreatment bone marrow blast % was 20% |
| | to <50%) |
| Rate of Stable Disease (SD) | Absence of CR_{MRD}, CR, CRᵢ, PR, MLFS, and |
| | criteria for PD not met |
| Time-to-event Endpoints: | OS: |
| Overall survival (OS) | Time to death from any cause, measured from |
| Event-free survival (EFS) | the date of entry into the trial to the date of death |
| Cumulative incidence of relapse | from any cause; patients not known to have died at |
| (CIR) | last follow-up are censored on the date they were last |
| | known to be alive |
| | EFS: |
| | Measured from the date of entry into the trial to |
| | the date of refractory disease, or relapse from CR or |
| | CRᵢ, or death from any cause |
| | CIR: |
| | Defined for all patients achieving CR or CRᵢ; |
| | measured from the date of achievement of a |
| | remission until the date of relapse; patients not |
| | known to have relapsed are censored on the date they |
| | were last examined; patients who died without |
| | relapse are counted as a competing cause of failure |
| Transfusion Independence: | Transfusion Independence for minimum of 8 |
| Rate of Transfusion | weeks |
| Independence | Overall duration of transfusion independence |
| Duration of Transfusion | |
| Independence | |

Hematological Improvement (HI)

| | |
|---|---|
| Erythroid (HI-E) | HI-E (pretreatment <11 g/dL): |
| Platelet (HI-P) | Hgb increased by ≥1.5 g/dL |
| Neutrophil (HI-N) | Relevant reductions of units of RBC |
| Proportion of patients who | transfusions by an absolute number of at least 4 RBC |
| achieve HI, transfusion independence | transfusions/8 weeks compared with pretreatment |
| or CR | transfusion number in the previous 8 weeks. Only |
| Duration of response (DoR) for | RBC transfusions given for an Hgb ≤9.0 g/dL |
| participants who achieved HI or better | pretreatment count in the RBC transfusion response |
| Percentage of patients with | evaluation |
| clinical benefit and/or absence of | HI-P (pretreatment <100 × 10⁹/L): |
| hematologic progression per | Absolute increase of ≥30 × 10⁹/L for patients |
| assessment | starting with >30 × 10⁹/L platelets |
| | Increase from <20 × 10⁹/L >20 × 10⁹/L and by |
| | at least 100% |
| | HI-N (pretreatment <1.0 × 10⁹/L): |
| | At least 100% increase and an absolute |
| | increase >0.5 × 10⁹/L |

MRD

| | |
|---|---|
| Rate of patients achieving MRD | MRD negativity |
| negative status | Calculated for patients MRD positive at |
| | baseline |

TABLE 4
MDS Efficacy Endpoints and Definitions in Part 1 and Part 2

| Efficacy Endpoint | Definition |
|---|---|
| Natural History | |
| ORR: (CR, Marrow CR) PR, | CR: |
| HI | Bone marrow ≤5% myeloblasts with normal maturation |
| | of all cell lines |
| | Persistent dysplasia is noted |
| | Hgb ≥11 g/dL |
| | Platelets ≥100 × 10⁹/L |
| | Neutrophils ≥1 × 10⁹/L |
| | Blasts 0% |
| | Marrow CR: |
| | Bone marrow ≤5% myeloblasts and ≥50% decrease |
| | over pretreatment |
| | Peripheral blood |
| | Marrow CR should be noted as a response with or |
| | without HI |
| | PR: |
| | All CR if abnormal before treatment except: |
| | Bone marrow blasts decreased by ≥50% over |
| | pretreatment but still >5% |
| | Cellularity and morphology not relevant |
| | HI: defined above |
| Rate of CR | Defined above |
| Rate of CR Marrow | Defined above |
| Rate of PR | Defined above |
| Rate of SD | Failure to achieve PR, but no evidence of progression |
| | for >8 weeks |
| Durability of response (CR, PR | Time from response to relapse |
| and morphologic complete | |
| remission (mCR) rate) | |
| Cytogenetic Response | Complete: |
| | For patients with chromosome abnormality, |
| | disappearance of the chromosome abnormality without |
| | appearance of new ones |
| | Response must last at least 4 weeks |
| | Partial: |
| | At least 50% reduction of the chromosome abnormality |
| | Response must last at 4 weeks |
| Survival Endpoints: | OS: |
| OS | The date of death from any cause, measured from the |
| EFS | date of entry into the trial to the date of death from any cause; |
| | patients not known to have died at last follow-up are censored |
| | on the date they were last known to be alive |
| | EFS: |
| | Measured from the date of entry into the trial to the date |
| | of treatment failure or death from any cause |
| Transfusion Independence | Transfusion independence for MDS patients |
| | Duration of transfusion independence minimum of 8 |
| | weeks |

Hematological Improvement

| | |
|---|---|
| Erythroid (HI-E) | HI-E (pretreatment <11 g/dL): |
| Platelet (HI-P) | Hgb increased by ≥1.5 g/dL |
| Neutrophil (HI-N) | Relevant reductions of units of RBC transfusions by an |
| Proportion of patients | absolute number of at least 4 RBC transfusions/8 weeks |
| who achieve HI, transfusion | compared with pretreatment transfusion number in the |
| independence or CR | previous 8 weeks. Only RBC transfusions given for an |
| DoR for participants | Hgb ≤9.0 g/dL pretreatment will count in the RBC transfusion |
| who achieved HI or better | response evaluation |
| Percentage of patients | HI-P (pretreatment <100 × 10⁹/L): |
| with clinical benefit and/or | Absolute increase of ≥30 × 10⁹/L for patients starting |
| absence of hematologic | with >30 × 10⁹/L platelets |
| progression per assessment | Increase from <20 × 10⁹/L to >20 × 10⁹/L and by at |
| | least 100% |
| | HI-N (pretreatment <1.0 × 10⁹/L): |
| | At least 100% increase and an absolute increase >0.5 × |
| | 10⁹/L |

### PK Endpoints in Part 1 and Part 2

The secondary PK endpoints are:
Evaluation of PK parameters of G9.2-17(IgG4) (including but not limited to, area under the curve from time zero until 168 h [AUC₀₋₁₆₈ₕ], maximum observed serum concentration [Cₘₐₓ], time to reach Cₘₐₓ [Tₘₐₓ]), and trough serum concentration [C_{trough}])

### Exploratory Endpoints in Part 1 and Part 2

TABLE 5
Exploratory Efficacy Endpoints: AML

| | |
|---|---|
| Rate of Progressive | PD: Evidence for increase in bone marrow blast percentage and/or |
| Disease (PD) | absolute increase in bast count in the blood: |
| | >50% increase in bone marrow blasts over baseline (a minimum |
| | 15% point increase is required in cases with <30% blasts at baseline), |
| | or persistent marrow blast percentage of >70% over at least 3 months, |
| | without at least a 100% improvement in ANC to an absolute level |
| | (>0.5 × 10⁹/L), and/or platelet count to >50 × 10⁹/L, non-transfused, or |
| | >50% increase in peripheral blasts (white blood cells [WBC] × % |
| | blasts) to >25 × 10⁹/L (in the absence of differentiation syndrome) |
| | New extramedullary disease |
| Rate of Treatment | Death in aplasia: |
| Failure: | Death occurring ≥7 days following completion of initial treatment |
| Death in | while cytopenic, with an aplastic or hypoplastic bone marrow obtained |
| aplasia | within 7 days of death, without evidence of persistent leukemia |
| Death from | Death from indeterminate cause: |
| indeterminate | Death occurring before completion of therapy, but no bone |
| cause | marrow examination available |
| | Death occurring ≥7 days following completion of initial therapy |
| | with no blasts in the blood, but no bone marrow examination available |
| Rate of Relapse | Molecular relapse |
| | Progression or relapse after HI: |
| | Proportion of patients who achieve HI, transfusion independence |
| | or CR |
| | DoR for participants who achieved HI or better |
| | Percentage of patients with clinical benefit and/or absence of |
| | hematologic progression per assessment |

TABLE 6
Exploratory Efficacy Endpoints: MDS

| | |
|---|---|
| Rate of PD | Patients with: |
| | <5% blasts: ≥50% increase in blasts to >5% blasts |
| | 5% to 10% blasts: ≥50% increase in blasts to >10% blasts |
| | 10% to 20% blasts: ≥50% increase in blasts to >20% blasts |
| | 20% to 30% blasts: ≥50% increase in blasts to >30% blasts |
| | Any of the following: |
| | At least 50% decrease from maximum remission/response in |
| | granulocytes or platelets |
| | Reduction in Hgb by >2 g/dL |
| | Transfusion dependence |
| Rate of | Death during treatment or disease progression characterized by |
| Treatment | worsening of cytopenia increase in percentage of bone marrow blasts, or |
| Failure | Progression to a more advanced MDS FAB subtype than pretreatment |
| | Primary refractory disease |
| | Death in aplasia |
| | Death from indeterminate cause |
| Rate of | Molecular relapse |
| Relapse | Progression or relapse after HI: |
| | Proportion of patients who achieve HI, transfusion independence or CR |
| | DoR for participants who achieved HI or better |
| | Percentage of patients with clinical benefit and/or absence of |
| | hematologic progression per assessment |

Exploratory Immunological and Molecular Changes in Peripheral Blood and Bone Marrow Endpoints:
Multiplexing of serum/plasma cytokines
Mass cytometry whole blood immune profiling
Gene array from whole blood
Exploratory Immunogenicity Endpoints
Evaluation of ADA

### III. Study Plan

This is an open-label, non-randomized, multi-center, Phase 1, single agent (Part 1) or combination agents (Part 2) dose escalation study in patients with AML whose disease is relapsed/refractory to at least one line of prior therapy, with or without an allogeneic stem cell transplant, or in patients with a documented diagnosis of relapsed/refractory, high-risk MDS relapsed/refractory to at least one line of treatment and for whom no standard therapy that may provide clinical benefit is available. The 4+2 algorithm-based dose-escalation design is used to establish DLTs and to help identify the RP2D (Lin et al., Biostatistics, 2001, 2, 203-15; Wheeler et al., PLoS One, 2016, 11, e0159026). A total of 5 planned dosage levels (Part 1 single agent) and 4 planned dosage levels (Part 2 combination agents) may be evaluated within the 4+2 design.

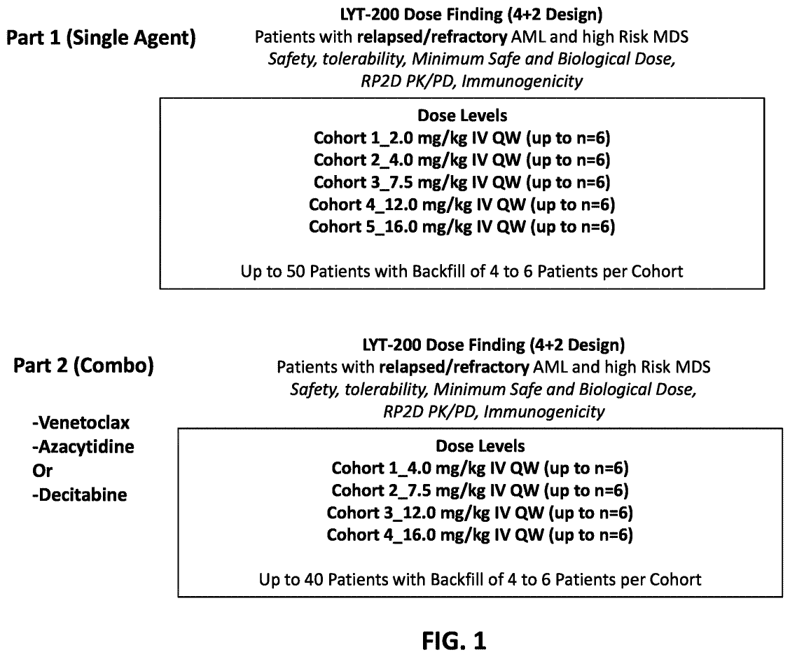

This study is conducted in approximately 10 sites in the United States. The study duration is estimated to be 24 months. Follow-up for survival continues for up to 2 years. The study design is outlined in FIG. 1, and the visit schedule and planned assessments at each visit are detailed in Table 7.

Dose Escalation and De-escalation
Treatment Period
The study consists of the following periods in both Part 1 and Part 2:

| | |
|---|---|
| Screening period: | Up to 2 weeks prior to first dose (Day −14 to Day −1) |
| Treatment period: | 28-day treatment cycles as presented in the Schedule |
| | of Assessments (SoA) (Table 7) |
| Post treatment | 30 days after last treatment (End of Treatment |
| period: | Visit/Early Termination Visit) |
| Follow-up period: | Long term follow-up for up to 2 years |
| | (every 3 months) |

### Part 1: Single Agent Dose Escalation

This dose-finding study is conducted using a 4+2 algorithm-based dose-escalation design to determine the MTD or the biologically active dose that is used to help identify the RP2D (Lin et al., Biostatistics, 2001, 2, 203-15; Wheeler et al., PLoS One, 2016, 11, e0159026). Four patients are initially enrolled at each dose level.

A total of 5 dose levels may be evaluated within the 4+2 design, with the starting dose of 2.0 mg/kg IV QW:
Dose Escalation Cohort 1=2.0 mg/kg IV QW (up to n=6)
Dose Escalation Cohort 2=4.0 mg/kg IV QW (up to n=6)
Dose Escalation Cohort 3=7.5 mg/kg IV QW (up to n=6)
Dose Escalation Cohort 4=12.0 mg/kg IV QW (up to n=6)
Dose Escalation Cohort 5=16.0 mg/kg IV QW (up to n=6)
One cycle (28 days) is 4 dose administrations of G9.2-17(IgG4).

If at any given dose level of single agent G9.2-17(IgG4), at least one patient of n=4 initially enrolled, or at least n=2 patients of n=6 enrolled (if in the first 4 patients there is a DLT), achieve at least stable disease based on bone marrow aspirate/biopsy analyses and/or hematological and/or clinical benefit (as per PI assessment), lasting for at least 2 cycles, then those patients may be intra-patient dose escalated to a higher dose level of G9.2-17(IgG4) than the one they enrolled into the study on, and up to the highest dose level of G9.2-17(IgG4) that has been declared safe and tolerated by the SMC, as per protocol.

TABLE 7
Schedule of Assessments

| | | Treatment Phase | | | | | | | | | | |
|---|---|---|---|---|---|---|---|---|---|---|---|---|
| | Pre-Dose | Cycle 1 | | | | | Cycle 2 | | | | Cycle 3 | |
| | Screening | (28 days) | | | | | (28 days) | | | | (28 days) | |
| | Study Day | | | | | | | | | | | |
| | −14 | | | | | | | | | | | |
| | to −1 | 1 | 2 | 8 | 15 | 22 | 1 | 8 | 15 | 22 | 1 | 8 |
| | Cycle Day | | | | | | | | | | | |
| | | C 1 | C 1 | C 1 | C 1 | C 1 | C 2 | C 2 | C 2 | C 2 | C 3 | C 3 |
| | | D 1 | D 2 | D 8 | D 15 | D 22 | D 1 | D 8 | D 15 | D 22 | D 1 | D 8 |
| +/−days | | | | 1 | 1 | 1 | 1 | 1 | 1 | 1 | 1 | 1 |
| allowable | | | | | | | | | | | | |
| Study drug | | X | | X | X | X | X | X | X | X | X | X |
| administration ^{A} | | | | | | | | | | | | |
| Eligibility | X | | | | | | | | | | | |
| assessment | | | | | | | | | | | | |
| and written | | | | | | | | | | | | |
| informed | | | | | | | | | | | | |
| consent | | | | | | | | | | | | |
| Demographics ^{B} | X | | | | | | | | | | | |
| Medical | X | | | | | | | | | | | |
| history ^{C} | | | | | | | | | | | | |
| Previous and | X | X | X | X | X | X | X | X | X | X | X | X |
| concomitant | | | | | | | | | | | | |
| medications ^{D} | | | | | | | | | | | | |
| AEs ^{E} | X | X | X | X | X | X | X | X | X | X | X | X |
| ECHO/MUGA ^{F} | X | | | | | | | | | | | |
| 12-lead ECG | X | | | | | | X | | | | | |
| Pre-dose only | | | | | | | | | | | | |
| Physical exam ^{G} | X | X | X | X | X | X | X | X | X | X | X | X |
| ECOG | X | X | | X | X | X | X | X | X | X | X | X |
| Vital Signs^{H} | X | X | X | X | X | X | X | X | X | X | X | X |
| Bone marrow | X | | | | | | X | | | | X | |
| aspiration^{I} | | | | | | | | | | | | |
| Pregnancy | X | | | | | | X | | | | X | |
| test ^{J} | | | | | | | | | | | | |
| Hematology ^{K} | X | X | X | X | X | X | X | X | X | X | X | X |
| Serum | X | X | X | X | X | X | X | X | X | X | X | X |
| Chemistry ^{L} | | | | | | | | | | | | |
| TSH, Free | X | | | | | | X | | | | X | |
| T4, serum | | | | | | | | | | | | |
| lipase, | | | | | | | | | | | | |
| amylase, | | | | | | | | | | | | |
| PTH, FSH, LH, | | | | | | | | | | | | |
| free cortisol | | | | | | | | | | | | |
| Blood | X | X | | X | X | X | X | X | X | X | X | X |
| Coagulation ^{M} | | | | | | | | | | | | |
| Urinalysis ^{N} | X | X | | | | | X | | | | X | |
| PD | | X | | | | | X | | | | X | |
| biomarkers | | | | | | | | | | | | |
| blood | | | | | | | | | | | | |
| sampling ^{O} | | | | | | | | | | | | |
| Hematology | | X | | | | | X | | | | X | |
| blood film | | | | | | | | | | | | |
| (local)^{P} | | | | | | | | | | | | |
| Central MRD | | X | | | | | X | | | | X | |
| blood | | | | | | | | | | | | |
| sampling ^{Q} | | | | | | | | | | | | |
| PK blood | | X | X | X | X | X | X | | | | X | X |
| sampling^{R} | | | | | | | | | | | | |
| Immunogenicity | | X | | | X | | X | | | | X | |
| (ADA)^{S} | | | | | | | | | | | | |
| Survival | | | | | | | | | | | | |
| Follow-up | | | | | | | | | | | | |

| | | | | | | | Post-Treatment | |
|---|---|---|---|---|---|---|---|---|
| | | | | | | | | Long |
| | | | | | | | | Term |
| | | | | | | | | Follow- |
| | Treatment Phase | | | | | | End of | Up (2 |
| | | | Cycle 4 | | | | Treatment | years) |
| | Cycle 3 | | (28 days) and | | | | or Early | Every 3 |
| | (28 days) | | beyond | | | | Termination | months |
| | Study Day | | | | | | | |
| | | | | | | | 30 days | |
| | | | | | | | after last | |
| | 15 | 22 | 1 | 8 | 15 | 22 | dose | |
| | Cycle Day | | | | | | | |
| | C 3 | C 3 | C 4 | C 4 | C 4 | C 4 | | |
| | D 15 | D 22 | D 1 | D 8 | D 15 | D 22 | — | — |
| +/−days | 1 | 1 | 1 | 1 | 1 | 1 | 3 | n/a |
| allowable | | | | | | | | |
| Study drug | X | X | X | X | X | X | | |
| administration ^{A} | | | | | | | | |
| Eligibility | | | | | | | | |
| assessment | | | | | | | | |
| and written | | | | | | | | |
| informed | | | | | | | | |
| consent | | | | | | | | |
| Demographics ^{B} | | | | | | | | |
| Medical | | | | | | | | |
| history ^{C} | | | | | | | | |
| Previous and | X | X | X | X | X | X | X | |
| concomitant | | | | | | | | |
| medications ^{D} | | | | | | | | |
| AEs ^{E} | X | X | X | X | X | X | X | |
| ECHO/MUGA ^{F} | | | X | | | | | |
| 12-lead ECG | | | | | | | | |
| Pre-dose only | | | | | | | | |
| Physical exam ^{G} | X | X | X | X | X | X | X | |
| ECOG | X | X | X | X | X | X | X | |
| Vital Signs^{H} | X | X | X | X | X | X | X | |
| Bone marrow | | | | | | | | |
| aspiration^{I} | | | | | | | | |
| Pregnancy | | | X | | | | X | |
| test ^{J} | | | | | | | | |
| Hematology ^{K} | X | X | X | X | X | X | X | |
| Serum | X | X | X | X | X | X | X | |
| Chemistry ^{L} | | | | | | | | |
| TSH, Free | | | X | | | | X | |
| T4, serum | | | | | | | | |
| lipase, | | | | | | | | |
| amylase, | | | | | | | | |
| PTH, FSH, LH, | | | | | | | | |
| free cortisol | | | | | | | | |
| Blood | X | X | X | X | X | X | X | |
| Coagulation ^{M} | | | | | | | | |
| Urinalysis ^{N} | | | X | | | | X | |
| PD | | | X | | | | | |
| biomarkers | | | | | | | | |
| blood | | | | | | | | |
| sampling ^{O} | | | | | | | | |
| Hematology | | | X | | | | X | |
| blood film | | | | | | | | |
| (local)^{P} | | | | | | | | |
| Central MRD | | | X | | | | X | |
| blood | | | | | | | | |
| sampling ^{Q} | | | | | | | | |
| PK blood | X | X | X | | | | | |
| sampling^{R} | | | | | | | | |
| Immunogenicity | | | X | | | | X | |
| (ADA)^{S} | | | | | | | | |
| Survival | | | | | | | | X |
| Follow-up | | | | | | | | |

ADA: anti-drug antibodies; AE: adverse event; ALT: alanine aminotransferase; APTT: activated partial thromboplastin time; AST: aspartate aminotransferase; C: cycle; CPK: creatine phosphokinase; COVID-19: Coronavirus SARS-CoV-2; CRP: C-reactive protein; D or d: day(s); ECG: electrocardiogram; ECOG: Eastern Cooperative Oncology Group; ECHO: echocardiography/cardiac ultrasound; FSH: follicle-stimulating hormone; IMAR: immune-mediated adverse reaction; INR: international normalized ratio; LDH: lactate dehydrogenase; LH: luteinizing hormone; min: minute(s); MRD; molecular residual disease: MUGA: multigated acquisition scan; PD: pharmacodynamics; PK: pharmacokinetics; PT: prothrombin time; PTH: parathyroid hormone; PTT: partial thromboplastin time; QTcF: QT interval, Fridericia's Correction Formula; RBC: red blood cell; SAE; serious adverse event; SGOT: serum glutamic-oxaloacetic transaminase; SGPT: serum glutamic pyruvic transaminase; TSH: thyroid stimulating hormone; WBC: white blood cell count
A) Study drug administration: G9.2-17(IgG4) treatment is administered on CXD1, CXD8, CXD15, and CXD22 on every weekly cycle. In the combination cohorts, venetoclax is administered daily, azacitidine is administered CXD1-7 (28-day cycles), decitabine is administered CXD1-5 (28-day cycles). See Dose Escalation and De-escalation for single agent and combination agent dosing information.
B) Demographics: Data include age, gender, race, and ethnicity.
C) Medical history: In addition to general medical history, data collection also includes oncology history, transfusion history within 8 weeks prior to study initiation surgical/transplant and radiation therapy history, and COVID-19 history and testing.
D) Previous and concomitant medications (including vaccines and complementary treatments/supplements): Data to include name, indication, dose, route, start and end dates for each. Allergies and intolerances and dose modifications while on study should also be obtained.
E) Adverse events: See Adverse Events.
F) ECHO/MUGA: This assessment of heart function is conducted at Screening and repeated on Day 1 of Cycle 4; the assessment window is +5 days. It should be conducted more frequently when clinically indicated and once every 3 months.
G) Physical exam: Include height at screening for determination of body surface area. Include weight at all scheduled exam times. A neurological exam is conducted only on patients who have stable and/or pre-treated brain metastases.
H) Vital Signs: temperature, heart rate, blood pressure, respiratory rate. Note: To monitor for infusion reactions on Cycle 1 Day 1, vital signs must be recorded every 15 minutes during the 1-hour infusion of G9.2-17(IgG4), and every 30 minutes through 1-hour post-infusion, and hourly thereafter if applicable.
I) Bone marrow aspiration: Monthly for the first 3 months (screening and then Day 1, Cycles 2 and 3 [+3 days]), then Q3M thereafter. Evaluated locally for disease response. Additionally, bone marrow aspirate to be sent to central lab for MRD analysis.
J) Pregnancy test (blood or urine): Only for women of childbearing potential with uterus in situ. Test results must be available before scheduled dosing.
K) Hematology: Analysis includes complete blood count, differential, platelets, hemoglobin. Collect blood samples pre-dose. Note: Cycle 1 Day 2 sampling has been added to provide for additional monitoring of TLS laboratory parameters.
L) Serum chemistry: Analysis includes glucose, total protein, albumin, electrolytes [sodium, potassium, chloride, magnesium, phosphorus], calcium, bilirubin (total, direct), SGPT (ALT) or SGOT (AST), alkaline phosphatase, (gamma GT, LDH, creatinine, HgbA1c (only if history of Type 1 or Type 2 diabetes mellitus and collected quarterly, more frequently if needed), blood urea nitrogen, and CPK. Fasting glucose to be assessed only if clinically indicated. Collect blood samples pre-dose. Note: Cycle 1 Day 2 sampling has been added to provide for additional monitoring of TLS laboratory parameters.
M) Blood Coagulation: Collect blood samples pre-dose. Analysis includes PT, PTT, and INR (if on allowable anticoagulants), CRP, and troponin.
N) Urinalysis: Analysis includes color, appearance, dipstick for specific gravity, protein, white blood cell-esterase, glucose, ketones, urobilinogen, nitrite, WBC, RBC, and pH (urine culture and sensitivity to be run only if patient is clinically symptomatic). Note: Urinalysis may be omitted on C1 D1 if collected during screening.

### O) PD Biomarkers: See Pharmacodynamic Biomarkers.

P) Hematology blood film (local): minimum assessments to capture: % of blasts. Additional assessments to capture if available: morphology of blasts with a particular focus on whether Auer rods are present, blood cell dysplasia.
Q) MRD assessment is conducted at the central lab.
R) PK blood sampling: Cycle 1 and Cycle 3 Day 1: blood samples are collected pre-dose, end of study drug infusion (EOI) and 1 h (+15 min) post-study drug administration. Cycle 1 Day 2 only a PK sample is also collected. Cycle 1 and Cycle 3 Day 8, Day 15, and Day 22, blood samples are collected pre-dose and at EOI only. Cycle 2 and every even cycle thereafter: blood samples are collected Day 1 only and should occur pre-dose and at EOI. Blood samples for PK are not collected on every odd cycle after Cycle 3. PK sampling may be updated based on emerging PK data in this study.
S) ADA blood sampling: Blood samples are collected pre-dose on Day 1 of every cycle and on Cycle 1 Day 15.

### Part 2: Combination Agents Dose Escalation

This dose-finding study is conducted using a 4+2 algorithm-based dose-escalation design to determine the MTD or the biologically active dose that is used to help identify the RP2D (Lin et al., Biostatistics, 2001, 2, 203-15; Wheeler et al., PLoS One, 2016, 11, e0159026). Four patients are initially enrolled at each dose level.

A total of 4 dose levels may be evaluated within the 4+2 design, with the starting dose of 4.0 mg/kg IV QW:
Dose Escalation Part 2 Cohort 1=4.0 mg/kg IV QW (up to n=6)
Dose Escalation Part 2 Cohort 2=7.5 mg/kg IV QW (up to n=6)
Dose Escalation Part 2 Cohort 3=12.0 mg/kg IV QW (up to n=6)
Dose Escalation Part 2 Cohort 4=16.0 mg/kg IV QW (up to n=6)
One cycle (28 days) is 4 dose administrations of G9.2-17(IgG4).
   Combination agent therapies in AML is as follows:
Oral venetoclax, Day 1, 100 mg, Day 2, 200 mg, Days 3-28, 400 mg
   One of the following HMAs:
Azacitidine, 75 mg/m² subcutaneously daily for 7 days (Days 1 through 7) in a 28-day cycle, or
Decitabine 20 mg/m² IV over 1 hour daily for 5 days (Days 1 through 5). Repeat cycles every 28 days
   Combination agent therapies in high-risk MDS are as follows:

One of the following HMAs:
Azacitidine, 75 mg/m² subcutaneously daily for 7 days (Days 1 through 7) in a 28-day cycle, or
Decitabine 20 mg/m² IV over 1 hour daily for 5 days (Days 1 through 5). Repeat cycles every 28 days

A cycle consists of 28 days. DLT period is 1 cycle, i.e. the first 28 days on study drugs. If during Cycle 1 or thereafter, in AML patients, venetoclax needs to be discontinued or dose modified, as per PIs assessment, because of venetoclax-related toxicities, patient are allowed to receive G9.2-17(IgG4)+HMA until their next bone marrow reassessment as per SoA or an unscheduled bone marrow reassessment, if required by the PI. If both venetoclax and HMA in AML patients or HMA in MDS patients need to be discontinued after Cycle 1 because of venetoclax/HMA-related toxicities, as per PI assessment, G9.2-17(IgG4) single agent can continue to be given only provided the PIs determine the patient may continue to derive clinical benefit from G9.2-17(IgG4) alone. G9.2-17(IgG4) related toxicity stopping rules apply throughout.

### Part 1 and Part 2: Dose Escalation, DLT, MTD, Biologically Effective Dose

Four patients are enrolled at each dose level. If 1 of 4 patients experiences a DLT, the dose level is expanded to a maximum of 6 patients. If ≥2 patients in a dose level of 4 to 6 patients experience a DLT, then the MTD is exceeded. The MTD is the highest dose at which 0-1 of 6 patients experience a DLT. In the absence of an observed MTD, treatment will continue until a biologically effective dose is observed.

The definition of a biologically effective dose is the dose at which 100% of patients in a cohort experience:
For AML: either MRD negative status or reduction in bone marrow blasts to ≤5% with absence of circulating blasts with Auer rods.
For MDS: either Transfusion independence duration minimum of 8 weeks or reduction in bone marrow myeloblasts to ≤5%.

In the absence of demonstrated clinical benefit, the time on study drug is limited to 6 cycles, i.e., 6 months. If a patient has not achieved a minimum of a PR as clinical benefit, the patient is taken off study drug, even in the absence of unacceptable toxicity. If a patient has demonstrated at least a PR as clinical benefit within 6 cycles (6 months) of starting study drug, that patient may continue dosing beyond 6 months until disease progression, patient withdrawal, or unacceptable toxicity.

Patients who withdraw for reasons other than toxicity or tolerability issues during the first treatment cycle only, are replaced.

### Part 1 and Part 2: Backfill Cohorts and Stopping Rules for Backfill Cohorts

Patient safety is established at a specific dose level prior to adding additional patients and/or allowing the backfill cohort to be opened.
If a DLT occurs in any patient during the first 28 days of treatment, that patient is permanently discontinued from study drug administration.
If 1 of 4 patients experiences a DLT, the dose level is expanded with 2 additional patients to a maximum of 6 patients. If ≥2 patients in a dose level of 4 to 6 patients experience a DLT, then the MTD is reached. The backfill cohort is not allowed to open at this dose level in this scenario.
If, however, any signs of clinical benefit are observed in any dosing cohort, determined based on secondary endpoints and exploratory endpoints, and the safety of that cohort has been established by not having observed a DLT in ≥2 patients in a dose level of 4 to 6 patients, a backfill of 4 to 6 patients within that dosing cohort may be added.
If a cohort is expanded to include backfill patients and 3 or more of the total patients at that dose level exhibit a DLT, then that dose level is the MTD, and the cohort is not expanded.
After the DLT period of 28 days, stopping rules for toxicity is guided by the G9.2-17(IgG4) dose modification/cessation guidelines presented in Table 8 (Management IMARS) and Table 9 (G9.2-17(IgG4) Dose Modifications).
For patients who experience toxicities, including IMARs outside of the DLT window, dose reduction is allowed only if clinical benefit is being derived and may continue to be derived at a lower dose level of G9.2-17(IgG4). The dose of G9.2-17(IgG4) is initially reduced by 50%, and potentially by a further reduction of 50%, as defined by the dose modification guidance provided in the protocol. No further dose reductions are allowed. In cases where patients withdraw for reasons other than toxicity or tolerability issues during the first treatment cycle only, they are replaced.

### Part 1 and Part 2: Dose De-Escalation (Cohort-1 Only)

In Part 1, during single agent Cycle 1 of Cohort 1 (2.0 mg/kg G9.2-17(IgG4)) only, a new Cohort-1 may be opened at a lower dose of G9.2-17(IgG4).

In Part 2, during combination agents Cycle 1 of Cohort 1 (4.0 mg/kg G9.2-17(IgG4)) only, a new Cohort-1 may be opened at a lower dose of 2.0 mg/kg G9.2-17(IgG4).

For both Part 1 and Part 2, the following applies:
If 2 or more patients reach DLT, then a lower dose Cohort-1 may be opened at a lower dose.
Q If any sign of clinical benefit is observed in Cohort 1 based on secondary, exploratory endpoints, or reductions in other markers of disease activity such as bone marrow blasts decreased by ≥50% over pretreatment, a lower dose Cohort 1 may be added for the purposes of exploring dose response relationships that will factor into dose selection for further development. Selection of the lower dose is based on available PK, PD data as well as markers of disease activity.

### Part 1 and Part 2: Completion of 4+2 Algorithm-Based Dose-Escalation

In both Part 1 and Part 2, the 4+2 algorithm-based dose-escalation is completed when one or more of the G9.2-17(IgG4) doses evaluated has been identified as the RP2D dose or doses.

### Dose-Limiting Toxicity Criteria

DLTs assessed in this trial are defined as a clinically significant hematologic and/or non-hematologic AE or abnormal laboratory value not clearly resulting from the underlying leukemia or extraneous causes occurring during the first cycle (28 days) on study and are at least possibly related to G9.2-17(IgG4). Any patient that experiences a DLT during the first 28 days of treatment is permanently discontinued from study drug administration.

A DLT is a toxicity that meets any of the following criteria:
Any death not clearly due to the underlying leukemia or extraneous causes
Indications of potential drug-induced liver injury (Hy's Law cases) as follows:
   Alanine aminotransferase (ALT) or AST >3× the upper limit of normal (ULN) with confirmation by repeat testing 24 hours later, AND
   Serum total bilirubin (TBL) >2×ULN with confirmation by repeat testing 24 hours later
   No other explanation can be found for the elevated TBL and/or aminotransferases (ATs), such as viral hepatitis (A, B, or C), alcoholic or autoimmune hepatitis, pre-existing or acute liver disease, gall bladder obstruction or bile duct disease, Gilbert syndrome, disease progression, or another medication capable of causing the observed effect
Grade ≥4 hematologic toxicities not clearly resulting from the underlying leukemia or extraneous causes, lasting >7 days. Events that exceed beyond the 28-day DLT monitoring period are also counted. All Grade 3-5 non-hematologic toxicity not clearly resulting from the underlying leukemia or extraneous causes, with the following exceptions:
   Grade 3 fatigue, asthenia, fever, anorexia (Grade 3 anorexia can be excluded only if it does not result in hospitalization, tube feeding, or use of total parenteral nutrition), or constipation
   Grade 3 nausea, vomiting or diarrhea not requiring tube feeding, total parenteral nutrition, or requiring or prolonging hospitalization
   Infection, bleeding, or other expected direct complication of cytopenias due to active underlying leukemia
   Grade 3 infusion reaction including CRS, if successfully managed and which resolves within 72 hours
   Grade 3 or 4 TLS if it is successfully managed clinically and resolves within 7 days without end-organ damage
   Grade 3 or 4 isolated electrolyte abnormalities that last <72 hours will not be considered a DLT

### End of Study for Part 1 and Part 2

End of study is defined at the point when the RP2D or doses have been identified, all patients have been treated with G9.2-17(IgG4) until confirmed disease progression or come off treatment for other reasons, and all patients have completed OS follow-up. Patients are followed for OS for up to 2 years following the last dose of G9.2-17(IgG4). The end of the study is defined as the date of the last patient's last visit.

### Trial Stopping Rules for Part 1 and Part 2

Dose-escalation phase of 4+2 design is completed when one of the doses from Cohorts 1 to 5 or intermittent dose has been identified as the RP2D. The RP2D is determined based on the MTD, DLTs, PK, PD, and additional safety data observed in each dose level and any other factors that might need to be considered. The trial is stopped for safety if the lower limit of an Agresti and Coull binomial CI for the lowest study dose level exceeds the target DLT rate (Agresti et al., Am Stat, 1998, 52, 119-126).

### IV. Study Population

Prospective approval of protocol deviations to recruitment and enrollment criteria, also known as protocol waivers or exemptions, is not permitted.

### Inclusion Criteria for Part 1 and Part 2

The following inclusion criteria must be met for a patient to be eligible for inclusion in the study:
1. Patients ≥18 years of age at the time of obtaining informed consent.
2. Patients with morphologically documented primary or secondary AML by the World Health Organization (WHO) criteria, whose disease is relapsed/refractory to at least one line of prior therapy, with or without an allogeneic stem cell transplant and for whom no standard therapy that may provide clinical benefit is available, or for patients who decline available standard of care.
3. Patients with a documented diagnosis of high-risk MDS, whose disease is relapsed/refractory post at least one line of treatment based on the revised International Prognostic Scoring System (IPSS-R), and for whom no standard therapy that may provide clinical benefit is available.
4. Patients are able to understand, sign, and date the written informed consent form (ICF) at screening visit prior to any protocol-specific procedures.
5. Patients are able and willing to comply with study procedures as per protocol (PP), including bone marrow biopsies.
6. Patient has an Eastern Cooperative Oncology Group (ECOG) performance status 2.
7. Patient's interval from prior treatment to time of study drug administration is at least 2 weeks, or at least 5 half-lives for agents. Upon discussion with the Medical Monitor, shorter than stated washout period may be considered provided that the patient has recovered from any clinically relevant safety issue and recovered to Grade ≤1 toxicity from prior therapies.
8. Patient must meet the following criteria as indicated on the clinical laboratory tests:
   WBC count at the time of the first dose of <25,000/uL.
   AST or ALT ≤3×ULN (≤5.0×ULN if considered to be due to leukemic involvement).
   Total bilirubin ≤2×ULN (≤3×ULN if considered to be due to leukemic involvement or Gilbert's syndrome).
   Creatinine clearance of ≥60 mL/min.
9. Patient has minimum life expectancy ≥3 months.
10. Female patient must be either: i) of non-child bearing potential: post-menopausal (defined as at least 1 year without any menses) prior to screening, or documented surgically sterile or status post hysterectomy (at least 1 month prior to screening); ii) if of childbearing potential, must have a negative serum or urine pregnancy test at screening, and must use 2 forms of birth control (at least one of which must be a barrier method) starting at screening and throughout the study period and for 90 days after the final study drug administration.
11. Female patient must not be breastfeeding at screening and during the study period, and for 90 days after the final study drug administration.
12. Female patient must not donate ova starting at screening and throughout the study period, and for 90 days after the final study drug administration.
13. Male patients and their female spouse/partners who are of childbearing potential must be using highly effective contraception consisting of 2 forms of birth control (one of which must be a barrier method) starting at screening.

### Exclusion Criteria for Part 1 and Part 2

A patient who meets any of the following exclusion criteria is not eligible for inclusion in the study:
1. Patient diagnosed with acute promyelocytic leukemia (APL).
2. Patient has active malignant tumors other than AML. Participants with a history of adequately treated malignancy including antecedent hematological malignancy for which no anticancer systemic therapy (namely chemotherapy, immunotherapy, small molecule inhibitor, radiotherapy, or surgery) is ongoing or required during the course of the study are eligible.
3. Patient has persistent non-hematological toxicities of ≥Grade 2 (CTCAE v5.0), with symptoms and objective findings from prior anti-cancer treatment (including chemotherapy, kinase inhibitors, immunotherapy, experimental agents, radiation, hematopoietic stem cell transplant [HSCT] or surgery).
4. Patient has had HSCT and meets any of the following: has undergone HSCT within the 6-month period prior to the first study dose; has ≥Grade 2 persistent non-hematological toxicity related to the transplant donor lymphocytes infusion.
5. Patient has active graft versus host disease (GVHD) and patients receiving immunosuppressive treatment for GVHD. Calcineurin inhibitors must be withdrawn for at least 4 weeks before Screening. Treatment with prednisolone 10 mg/daily p.o. or equivalent is allowed.
6. Patient with symptomatic central nervous system (CNS) involvement of leukemia or other CNS diseases related to underlying and secondary effects of malignancy.
7. Known inherited or acquired bleeding disorders and/or active bleeding, including disseminated intravascular coagulation abnormality (DIC).
8. Patient has had major surgery within 4 weeks prior to the first study dose. Consult with Medical Monitor to assess determination of major surgery if not clear.
9. Patient has had radiation therapy within 4 weeks prior to the first study dose.
10. Patient has congestive heart failure New York Heart Association (NYHA) class 3 or 4, or patient with a history of congestive heart failure NYHA class 3 or 4 in the past, unless a screening echocardiogram or multigated acquisition (MUGA) scan performed within 3 months prior to study entry results in a left ventricular ejection fraction (LVEF) that is ≥45%.
11. Patient has any clinically important abnormalities in: i) rhythm; ii) conduction; iii) morphology on the resting ECG (e.g., complete left bundle bunch block, third degree heart block, second degree heart block, PR interval >250 msec); and/or IV) any factors that increase the risk of QTc prolongation or risk of arrhythmic events, such as congenital long QT syndrome or family history of long QT syndrome, that are medically unmanaged.
12. Patient is known to have serious symptomatic active infection. Upon review with Medical Monitor, well controlled active infection may be permitted.
13. Patient has known active hepatitis B or C, or other active hepatic disorder.
14. Patient has any condition which makes the patient unsuitable for study participation.
15. Patients unwilling or unable to comply with the protocol.
16. Live vaccine administered within 30 days prior to start of treatment.
17. History of severe hypersensitivity reactions to any monoclonal antibodies (mAbs) or any of the combination therapies in Part 2 and/or their excipients.
18. History of pulmonary embolism, stroke, or transient ischemic attack within 2 months prior to Cycle 1, Day 1.
19. Drug abuse or long-term alcohol abuse that would affect the evaluation results.
20. Active autoimmune disease requiring systemic therapy (except autoimmune hypothyroidism treatable with synthetic thyroid hormone).

### V. Description of Study Assessments (Part 1 and Part 2)

### Demographics and Other Screening Assessments

At screening, patient demographic data is collected. These include age, gender, race, and ethnicity. Safety assessments that are also part of the screening assessments are described herein.

### Medical History

The medical history includes oncology history, transfusion history within 8 weeks prior to study initiation, surgical/transplant history, radiation therapy history, and COVID-19 history and testing.
Personal medical history, including prior treatments/surgeries, record of any implants in situ or past implants, prior and/or current use of medical devices, concomitant medications (name, indication, dose, route, start and end dates dose modifications if any and reason), pre-existing symptoms and AEs, hereditary diseases at risk of based on family history and complete family history to the best knowledge of the patient
Record of any dental work performed in the past 12 months
For patients with previously resected pancreatic adenocarcinoma, record whether the primary tumor was localized to the head of pancreas, pancreatic body or the pancreatic tail
Bowel habits/typical frequency and consistency
Record of any dietary requirements or preferences (for example, practice of a particular diet regimen: intermittent fasting, keto diet, etc.)
Record of any allergies past and present (allergen, severity)

### ECHO/MUGA

ECHO and/or MUGA is obtained at the timepoints indicated in the SoA (Table 7). If clinically indicated, the assessment is to be repeated once every 3 months.

### ECOG

ECOG performance status is assessed at the timepoints indicated in the SoA (Table 7) using the following grading (Oken et al., Am J Clin Oncol, 1982, 5, 649-655):
Grade 0: Fully active, able to carry on all pre-disease performance without restriction
Grade 1: Restricted in physically strenuous activity but ambulatory and able to carry out work of a light or sedentary nature, e.g., light housework, office work
Grade 2: Ambulatory and capable of all self-care but unable to carry out any work activities. Up and about more than 50% of waking hours
Grade 3: Capable of only limited self-care, confined to bed or chair more than 50% of waking hours
Grade 4: Completely disabled. Cannot carry on any self-care. Totally confined to bed or chair
Grade 5: Dead

### Efficacy Assessments

### Efficacy Assessments (Secondary)

Efficacy assessments are derived from updated International Working group (IWG) and key opinion leader recommendations for AML and MDS (Dohner et al., Blood, 2017, 129, 424-448; Cheson et al., Blood, 2006, 108, 419-425).

Efficacy endpoints and definitions are located in Table 3 for AML and Table 4 for MDS.

All endpoints for both AML and MDS are derived from 4 types of assessment, collected according to the SoA (Table 7):
Hematology (routine component of clinical laboratory evaluation,)
MRD
Bone marrow aspirate, and
Hematology blood smears.

### Molecular Residual Disease (MRD)

MRD comprises central multiparameter flow cytometry (MFC) in bone marrow for immunophenotype markers and is conducted according to the SoA (Table 7). This is evaluated at the central lab.

### Bone Marrow Aspirate

Bone marrow aspirates are evaluated locally for disease response. Additionally, bone marrow aspirate is sent to the central lab for MRD analysis by RT-qPCR/NGS molecular genetic analysis, and may also include MFC, according to the SoA (Table 7).

Note: After Cycle 3, it is allowed to not complete the bone marrow aspirate if not medically feasible, in consultation with the Medical Monitor.

### Hematology Blood Film

The hematology blood film using local laboratory standards/methods to analyze:
% of blasts
If available with local laboratory capabilities, to assess morphology of blasts with a particular focus on whether Auer rods are present (which is typical for AML)
If available with local laboratory capabilities, to assess blood cell dysplasia Hematology blood films are conducted locally according to the SoA (Table 7).

### Safety Assessments

### Adverse Events

Described herein.

### Clinical Laboratory Evaluations

Patients have blood samples collected for routine clinical laboratory testing (approximately 5 mL at each timepoint), according to the SoA (Table 7); additional tests may be performed at any time during the study as determined necessary or required by local regulations. Protocol-specific requirements for inclusion or exclusion of patients are detailed in Section 5 of the protocol.

The clinical laboratory parameters are analyzed at the site's local laboratory. Laboratory assessments to be completed include hematology and serum chemistry and are defined as following:
Serum Chemistry: To include glucose, total protein, albumin, electrolytes [sodium, potassium, chloride, magnesium, phosphorus], calcium, bilirubin (total, direct), SGPT (ALT) or SGOT (AST), alkaline phosphatase, gamma glutamyl transferase (gamma GT), lactate dehydrogenase (LDH), creatinine, hemoglobin A1c (HgbA1c) (only if history of Type 1 or Type 2 diabetes mellitus and collected quarterly, more frequently if needed), blood urea nitrogen, creatine phosphokinase (CPK)
   Thyroid stimulating hormone (TSH), fT4, lipase, amylase, parathyroid hormone (PTH), follicle-stimulating hormone (FSH), luteinizing hormone (LH), free cortisol additionally at specified visits
   Fasting glucose is assessed only if clinically indicated
Hematology: To include complete blood count (WBCs, RBCs, platelets, hemoglobin), hematocrit, differential (neutrophils, lymphocytes, monocytes, eosinophils, basophils)
Coagulation: To include PT and partial thromboplastin time (PTT) and international normalized ratio (INR) (if on allowable anticoagulants) C-reactive protein (CRP), and troponin
Urinalysis: Patients have urine samples collected for routine urinalysis. The urinalysis includes color, appearance, and dipstick for specific gravity, protein, white blood cell-esterase, glucose, ketones, urobilinogen, nitrite, WBC, RBC, and pH, and urine culture (if patient is clinically symptomatic).

The laboratory report must be reviewed, the review documented, and any clinically significant changes occurring during the study as an AE recorded. The laboratory reports must be filed with the source documents.

Abnormal laboratory findings associated with the underlying disease are not considered clinically significant unless deemed to be more severe than expected for the participant's condition.

All laboratory tests with values considered clinically significantly abnormal during participation in the study or within 30 days after the last dose of study drug should be repeated until the values return to normal or baseline or are no longer considered clinically significant. If clinically significant values do not return to normal/baseline or Grade 1 within a period of time, the etiology should be identified.

All protocol-required laboratory tests must be conducted in accordance with the laboratory manual and the SoA (Table 7).

If laboratory values from non-protocol-specified laboratory tests performed at the institution's local laboratory require a change in participant management or are considered clinically significant (e.g., SAE or AE or dose modification), then the results must be recorded.

### 12-Lead Electrocardiogram

12-lead ECG is obtained as outlined in the SoA (Table 7) using an ECG machine that automatically calculates the heart rate and measures heart rate, PR interval, QRS duration, distance in time on the ECG tracing from the start of the QRS complex to the end of T-wave (QT) interval, and QTcF intervals.

### Vital Signs

Vital signs are measured in a post-supine position after 5 minutes rest and include temperature, blood pressure (systolic and diastolic), heart rate, and respiratory rate, according the SoA (Table 7).

Note: To monitor for G9.2-17(IgG4) infusion reactions on Cycle 1 Day 1, vital signs must be recorded every 15 minutes during the 1-hour infusion of G9.2-17(IgG4), and every 30 minutes through 1-hour post-infusion, and hourly thereafter if applicable.

### Physical Examination

Medical and physical examinations must be performed by a qualified physician, nurse practitioner, or physician assistant, and should include a thorough review of all body systems. Additionally, height is measured (screening only for determination of body surface area). Include weight at all scheduled exam times. A neurological exam is conducted only on patients who have stable and/or pre-treated brain metastases.

### Monitoring and Treatment for Tumor Lysis Syndrome

As described herein, the following precautions are implemented to monitor and treat potential TLS:
In order to provide for adequate monitoring of laboratory parameters associated with TLS during the first week of G9.2-17(IgG4) treatment, in addition to the scheduled blood draw on Cycle D1 Day 1, a second blood draw is collected on Cycle 1 Day 2.
Patients may be risk assessed for TLS prior to each G9.2-17(IgG4) infusion and, where clinically indicated (e.g., TLS experienced with prior therapy regimens), treated according to institutional standard of care, such as with adequate hydration and uricostatics prior to the start of the G9.2-17(IgG4) infusion.
Where a patient has a high disease burden and is at greater risk of TLS, it may also be appropriate to keep/admit them for additional monitoring following G9.2-17(IgG4) administration.
   TLS should be treated according to standard institutional practice.

### Pharmacokinetics

The following serum PK parameters are calculated for G9.2-17(IgG4), if possible:
AUC₀₋₁₆₈ₕ
Cₘₐₓ
Tₘₐₓ
Serum concentration vs. time profiles

Blood samples of approximately 5 mL are collected and processed to serum at each timepoint as specified in the SoA (Table 7). The actual date and time of collection of each sample is recorded.

A complete list of PK parameters is provided in the statistical analysis plan (SAP).

### Pharmacodynamic Biomarkers

Planned time points for biomarker assessments are provided in the SoA (Table 7); sampling may be decreased to every third cycle after 6 months of treatment.

The following samples for biomarker research are required and are collected from all participants in this study as specified in the SoA.
Blood samples, to be collected prior to study drug administration (approximately 15 mL pre-dose)
Samples are tested for PD biomarkers (by flow cytometry, enzyme-linked immunosorbent assay (ELISA), IHC, and/or multiplex phenotyping) to evaluate their association with the observed clinical responses to G9.2-17(IgG4) and G9.2-17(IgG4)+venetoclax and/or HMA, using validated assays. Samples are stored at the central laboratory and analyzed if analysis is determined to be appropriate to support other study findings.

The following biomarkers are planned to be assessed for in this study (note the listing below includes expected biomarkers but are not limited to only these):
Multiplexing of serum/plasma cytokines
Mass cytometry whole blood immune profiling
Gene array from whole blood

Exploratory biomarker changes, if any, are correlated with safety and response outcomes. Samples may be stored for a maximum of 2 years (or according to local regulations) following the last patient's last visit for the study to enable further analysis of the effect of G9.2-17(IgG4) on PD biomarkers.

### Immunogenicity Assessments

Blood samples (approximately 3 mL) are collected from all participants according to the SoA (Table 7) and processed to serum. Additionally, serum samples should also be collected at the End of Treatment/Early Termination visit from patients who discontinued study drug or were withdrawn from the study.

### VI. Study Drugs

### G9.2-17(IgG4)

All patients receive G9.2-17(IgG4). G9.2-17(IgG4) is administered via IV infusion QW over approximately 60 minutes. Patients receive G9.2-17(IgG4) as a single agent at sequentially increasing doses starting at 2.0 mg/kg, in accordance with the 4+2 design (described herein). Patients who experience a DLT in Part 1 do not resume treatment.

### G9.2-17(IgG4): Preparation, Handling, Storage, and Accountability

G9.2-17(IgG4) is supplied in single use vials. It must be confirmed that appropriate temperature conditions have been maintained during transit for all study drug received and any discrepancies are reported and resolved before use of the study drug.

Only patients enrolled in the study may receive study drug and only authorized site staff may supply or administer study drug. All study drug must be stored in a secure, environmentally controlled, and monitored (manual or automated) area in accordance with the labeled storage conditions.

### G9.2-17(IgG4): Preparation

Manufacture and packaging of the investigational medicinal product (IMP) G9.2-17(IgG4) is in accordance with applicable current Good Manufacturing Practice (cGMP) and the product meets applicable criteria for use in humans.

G9.2-17(IgG4) liquid drug product is diluted to the target dose prior to administration. All dilutions should be performed in a controlled and sterile environment (patient dose is prepared for and delivered via an approximately 60 minutes IV infusion).

### G9.2-17(IgG4): Dose and Administration

All patients receive G9.2-17(IgG4). G9.2-17(IgG4) is administered via IV infusion for approximately 60 minutes, QW. The infusion start and duration of infusion is recorded. Patients receive G9.2-17(IgG4) as a single agent at sequentially increasing doses starting at 2.0 mg/kg, in accordance with the 4+2 design (described herein). Patients who experience a DLT in Part 1 do not resume treatment.

### G9.2-17(IgG4): Dose Administration

If an IRR is encountered, interrupt the infusion and if clinically indicated, administer relevant medication(s) (e.g., anti-histamine, anti-emetic, steroids, anti-pyretics, beta-blocker(s), etc.). If it is deemed appropriate to resume the infusion, resume at a slower infusion rate. Upon re-initiation of infusion, the new rate, as well as the stop and restart times for the infusion are recorded.

For subsequent cycles for the same patient, apply the appropriate pre-medications (anti-histamine, anti-emetic, steroids, anti-pyretics, beta-blocker(s), etc., as clinically indicated) and consider utilizing a slower infusion rate.

### G9.2-17(IgG4): Dose Modification

The decision to proceed to the next dose level of G9.2-17(IgG4) is made based on safety, tolerability, and potential preliminary PK data obtained in at least 4 patients at the prior dose level. The dosing schedule may also be adjusted based on PK data obtained.

Detailed dose modification instructions are available as follows:

TABLE 8
Management of IMARs caused by G9.2-17(IgG4)

| IMAR | Guidance for Management |
|---|---|
| Immune-mediated | Monitor patients for signs with radiographic imaging and for clinical |
| pneumonitis | symptoms of pneumonitis. |
| | Withhold G9.2-17(IgG4) for moderate and permanently discontinue |
| | for severe or life-threatening pneumonitis. |
| | Administer corticosteroids at a dose of 1 to 2 mg/kg/day prednisone |
| | equivalents for moderate (Grade 2) or more severe (Grade 3-4) |
| | pneumonitis, followed by corticosteroid taper. |
| | Withhold G9.2-17(IgG4) until resolution to <Grade 2. For moderate |
| | (Grade 2) pneumonitis. Resume treatment at a 50% dose reduction. |
| | Upon recurrence of Grade 2 pneumonitis at the reduced dose of |
| | G9.2-17(IgG4), permanently discontinue G9.2-17(IgG4). |
| Immune-mediated | Monitor patients for signs and symptoms of colitis. |
| colitis | Administer corticosteroids at a dose of 0.5 to 1 mg/kg/day |
| | prednisone equivalents followed by corticosteroid taper for moderate |
| | (Grade 2) colitis of more than 5 days duration. |
| | If worsening or no improvement occurs despite initiation of |
| | corticosteroids, increase dose to 1 to 2 mg/kg/day prednisone |
| | equivalents. |
| | When initial Grade 2 colitis resolves to <Grade 2, resume G9.2- |
| | 17(IgG4) at a 50% dose reduction. |
| | If³ Grade 2 colitis recurs, withhold G9.2-17(IgG4) until colitis |
| | resolves to <Grade 2. Resume G9.2-17(IgG4) at an additional 50% |
| | dose reduction. No further dose reductions for G9.2-17(IgG4) are |
| | allowed. |
| | Administer corticosteroids at a dose of 1 to 2 mg/kg/day prednisone |
| | equivalents followed by corticosteroid taper for severe (Grade 3) or |
| | life-threatening (Grade 4) colitis. |
| | When Grade 3 colitis resolves to ≤Grade 2, resume G9.2-17(IgG4) |
| | at a 50% dose reduction. |
| | Permanently discontinue G9.2-17(IgG4) for life-threatening (Grade |
| | 4) or for recurrent colitis of ≥Grade 2 upon re-initiation of G9.2- |
| | 17(IgG4). |
| Immune-mediated | Monitor for changes in liver function. |
| hepatitis | Administer corticosteroids at a dose of 0.5 to 1 mg/kg/day |
| | prednisone equivalents for moderate (Grade 2) transaminase |
| | elevations. |
| | Withhold G9.2-17(IgG4) for moderate (Grade 2) immune-mediated |
| | hepatitis. When resolved to <Grade 2 resume G9.2-17(IgG4) at a |
| | 50% dose reduction. |
| | If ≥Grade 2 hepatitis recurs, permanently discontinue G9.2- |
| | 17(IgG4). |
| | Administer corticosteroids at a dose of 1 to 2 mg/kg/day prednisone |
| | equivalents followed by corticosteroid taper for severe (Grade 3) or |
| | life-threatening (Grade 4) transaminase elevations, with or without |
| | concomitant elevation in total bilirubin. |
| | Permanently discontinue G9.2-17(IgG4) for severe (Grade 3) or life- |
| | threatening (Grade 4) immune-mediated hepatitis. |
| Immune-mediated | Administer hormone replacement as clinically indicated and |
| endocrinopathies: | corticosteroids at a dose of 1 mg/kg/day prednisone equivalents |
| Hypophysitis | followed by corticosteroid taper for moderate (Grade 2) or greater |
| | hypophysitis. |
| | Withhold G9.2-17(IgG4) for moderate (Grade 2) or severe (Grade 3) |
| | and when resolved to <Grade 2, resume G9.2-17(IgG4) at a 50% |
| | dose reduction. |
| | If ≥Grade 2 toxicity recurs, permanently discontinue G9.2-17(IgG4). |
| | Permanently discontinue G9.2-17(IgG4) for life-threatening (Grade |
| | 4) hypophysitis. |
| Adrenal insufficiency | Monitor patients for signs and symptoms of adrenal insufficiency. |
| | Withhold G9.2-17(IgG4) for moderate (Grade 2) and once resolved |
| | to <Grade 2, resume G9.2-17(IgG4) at a 50% dose reduction. |
| | If ≥Grade 2 toxicity recurs, permanently discontinue G9.2-17(IgG4). |
| | Administer corticosteroids at a dose of 1 to 2 mg/kg/day prednisone |
| | equivalents followed by a corticosteroid taper for severe (Grade 3) or |
| | life-threatening (Grade 4) adrenal insufficiency. |
| | Permanently discontinue G9.2-17(IgG4) for severe (Grade 3) or life- |
| | threatening (Grade 4) adrenal insufficiency, or for recurrent colitis |
| | of ≥Grade 2 upon re-initiation of G9.2-17(IgG4). |
| Hypothyroidism and | Monitor thyroid function prior to and periodically during |
| Hyperthyroidism | tislelizumab treatment. |
| | Administer hormone replacement therapy for hypothyroidism. |
| | Initiate medical management for control of hyperthyroidism. |
| | There are no recommended dose adjustments of G9.2-17(IgG4) |
| | for <Grade 3 hypothyroidism or hyperthyroidism. |
| | Discontinue G9.2-17(IgG4) for ≥Grade 3 hypothyroidism or |
| | hyperthyroidism. |
| Type 1 Diabetes | Withhold G9.2-17(IgG4) in cases of severe (Grade 3) hyperglycemia |
| Mellitus | until metabolic control is achieved. |
| | Upon resolution to ≤Grade 2, resume G9.2-17(IgG4) at a 50% dose |
| | reduction. |
| | If ≥Grade 2 toxicity recurs, permanently discontinue G9.2-17(IgG4). |
| | Permanently discontinue G9.2-17(IgG4) for life-threatening (Grade |
| | 4) hyperglycemia. |
| Immune-mediated | Monitor patients for elevated serum creatinine prior to and |
| nephritis and renal | periodically during treatment. |
| dysfunction | Administer corticosteroids at a dose of 0.5 to 1 mg/kg/day |
| (Defined as renal | prednisone equivalents for moderate (Grade 2) or severe (Grade 3) |
| dysfunction or ≥Grade | increased serum creatinine, if worsening or no improvement occurs, |
| 2 increased creatinine, | increase dose of corticosteroids to 1 to 2 mg/kg/day prednisone |
| requirement for | equivalents. |
| corticosteroids, and no | Withhold G9.2-17(IgG4) for moderate (Grade 2) or severe (Grade 3) |
| clear alternate | increased serum creatinine. When resolved to <Grade 2, resume |
| etiology) | G9.2-17(IgG4) at a 50% dose reduction. |
| | If ≥Grade 2 toxicity recurs, permanently discontinue G9.2-17(IgG4). |
| | Administer corticosteroids at a dose of 1 to 2 mg/kg/day prednisone |
| | equivalents followed by corticosteroid taper for life-threatening |
| | (Grade 4) increased serum creatinine. |
| | Permanently discontinue G9.2-17(IgG4) for life-threatening (Grade |
| | 4) increased serum creatinine. |
| Immune-mediated skin | Withhold for severe and permanently discontinue for life-threatening |
| adverse reactions | rash. |
| (Immune-mediated | For symptoms or signs of SJS or TEN, withhold G9.2-17(IgG4) and |
| rash, including | refer the patient for specialized care for assessment and treatment. |
| Stevens-Johnson | If SJS or TEN is confirmed, permanently discontinue G9.2-17(IgG4). |
| syndrome [SJS] and | For immune-mediated rash ≥Grade 2, administer corticosteroids at a |
| toxic epidermal | dose of 1 to 2 mg/kg/day prednisone equivalents followed by a |
| necrolysis [TEN]) | corticosteroid taper for severe (Grade 3) or life-threatening (Grade 4) |
| | rash. |
| | Withhold G9.2-17(IgG4) for moderate (Grade 2) and severe (Grade |
| | 3) rash and when resolved to <Grade 2, resume G9.2-17(IgG4) at a |
| | 50% dose reduction. |
| | If ≥Grade 2 rash recurs, withhold G9.2-17(IgG4) until resolved |
| | to <Grade 2 and resume G9.2-17(IgG4) at an additional 50% dose |
| | reduction. No further dose reductions are allowed. |
| | Permanently discontinue G9.2-17(IgG4) for life-threatening (Grade |
| | 4) rash. |
| Immune-mediated | Monitor for changes in neurologic function. |
| encephalitis | Evaluation of patients with neurologic symptoms may include, but |
| | not be limited to, consultation with a neurologist, brain MRI, and |
| | lumbar puncture. |
| | Withhold G9.2-17(IgG4) in patients with new-onset moderate to |
| | severe neurologic signs or symptoms and evaluate to rule out |
| | infectious or other causes of moderate to severe neurologic |
| | deterioration. |
| | If other etiologies are ruled out, administer corticosteroids at a dose |
| | of 1 to 2 mg/kg/day prednisone equivalents for patients with |
| | immune-mediated encephalitis, followed by corticosteroid taper. |
| | Permanently discontinue G9.2-17(IgG4) for immune-mediated |
| | encephalitis. |
| Other IMARs | If uveitis occurs in combination with other IMARs, consider a Vogt- |
| (May include: | Koyanagi-Harada-like syndrome, and may require treatment with |
| myocarditis, | systemic steroids to reduce the risk of permanent vision loss. |
| rhabdomyolysis, | For any suspected IMARs, exclude other causes. |
| myositis, uveitis, iritis, | Based on the severity (Grade) of the adverse reaction, permanently |
| pancreatitis, facial and | discontinue or withhold G9.2-17(IgG4), administer high-dose |
| abducens nerve paresis, | corticosteroids, and if appropriate, initiate hormone replacement |
| demyelination, | therapy. |
| polymyalgia | Upon improvement to Grade 1 or less, initiate corticosteroid taper |
| rheumatica, | and continue to taper over at least 1 month. |
| autoimmune | Consider restarting G9.2-17(IgG4) after completion of corticosteroid |
| neuropathy, Guillain- | taper based on the severity of the event, resuming at a 50% dose |
| Barré syndrome, | reduction. |
| hypopituitarism, | |
| systemic inflammatory | |
| response syndrome, | |
| gastritis, duodenitis, | |
| sarcoidosis, histiocytic | |
| necrotizing | |
| lymphadenitis (Kikuchi | |
| lymphadenitis), motor | |
| dysfunction, vasculitis, | |
| aplastic anemia, | |
| pericarditis, and | |
| myasthenic syndrome) | |

TABLE 9
Recommended Dose Modifications for G9.2-17(IgG4)

| Adverse Reaction | Severity | G9.2-17(IgG4) Dose Modification |
|---|---|---|
| Non-hematologic | ≥Grade 4 | Permanently discontinue treatment |
| and hematologic | | with G9.2-17(IgG4). |
| toxicities | | |
| Hematologic | Grade 3 | No dose reduction. |
| toxicities | | |
| Non-hematologic | Grade 3 | Withhold treatment until toxicity |
| toxicities | | resolves to ≤Grade 2. |
| (except those listed | | Resume G9.2-17(IgG4) at 50% |
| in Table 8 | | reduction from previous dose. |
| (Management of | | If recurs again at ≥Grade 3 severity, |
| IMARS), | | hold until resolved to ≤Grade 2 and |
| Management of | | implement a further 50% dose |
| IMARs Caused by | | reduction. |
| G9.2-17(IgG4)) | | No further dose reductions are |
| | | allowed. |
| Gastrointestinal | Grade 3 | Withhold G9.2-17(IgG4) until toxicity |
| (Nausea, Vomiting) | Not requiring hospitalization or | reverses to ≤Grade 2 |
| | parenteral nutrition support; | Resume G9.2-17(IgG4) with no dose |
| | managed by supportive care | reduction |
| Electrolyte | Grade 3 corrected to ≤Grade 2 | If recurrent, withhold G9.2-17(IgG4) |
| Abnormalities | within 24 hours | until toxicity reverses to ≤Grade 2 |
| Electrolyte | ≥Grade 3 lasting <24-72 hours | Then resume G9.2-17(IgG4) at a 50% |
| Abnormalities | Not clinically complicated; | reduction from the previous dose |
| | resolves spontaneously or | If recurrent, withhold G9.2-17(IgG4) |
| | responds to conventional | until toxicity reverses to ≤Grade 2 |
| | medical interventions | Resume G9.2-17(IgG4) at an |
| Amylase or Lipase | ≥Grade 3 | additional 50% dose reduction |
| Elevation | Not associated with symptoms or | No further dose reductions are allowed |
| | clinical manifestations of | |
| | pancreatitis | |

(for AEs outside the DLT window and other than IMARs)

### G9.2-17(IgG4): Dose Delay

If any clinically meaningful AE of Grade ≥3 possibly related or related to one or more study drugs occurs, it is discussed before continuing with dosing. A dose delay may be necessary for ≥Grade 3 AE.

### G9.2-17(IgG4): Dose Reduction

No dose reductions are allowed for any patient who is being evaluated for DLTs (within the 28-day DLT window). Dose reduction is allowed only if clinical benefit is being derived and may continue to be derived under dose reduced conditions; see Table 8 (IMAR Management) (for IMARs) or Table 9 (LYTDose mods) (for other AEs).

### G9.2-17(IgG4): Dose Modification for IMARs

If an IMAR occurs, refer to Table 8 (IMAR Management) for guidance on dose management of G9.2-17(IgG4). All relevant medical exam(s)/test(s) should be carried out in order to confirm that the AE is an IMAR.

Agents Administered in Combination with G9.2-17(IgG4)

Compared to frontline treatment, limited data of HMA-VEN combination exist on its effect in the relapsed/refractory (r/r) setting. Equally, VEN+HMA is approved in frontline treatment context, therefore official frequencies of toxicities in R/R setting are discernable from investigational studies and from real-world evidence, which is still sparce. It is expected that in the r/r patient population, the frequency of toxicities, across all types and grades may be considerably higher than in frontline patients, in AML and MDS.

Because clinical trials are conducted under widely varying conditions, adverse reaction rates observed in the clinical trials of a drug cannot be directly compared to rates in the clinical trials of another drug and may not reflect the rates observed in practice.

### (A). Bcl-2 Inhibitor: Venetoclax

Venetoclax (VENCLEXTA®) is a bcl-2 (B-cell lymphoma 2) inhibitor, and it helps restore the process of apoptosis in AML cells. Approximately 80% of patients with AML overexpress a bcl-2 pro-survival protein. Initiation of venetoclax in patients with AML does not require biomarker or cytogenetic testing results.

### (i). Venetoclax as Monotherapy

The most common adverse reactions (≥20%) of any grade were neutropenia, diarrhea, nausea, anemia, thrombocytopenia, fatigue, upper respiratory tract infection, and cough. Serious adverse reactions were reported in 56.5% of patients. The most frequently reported serious adverse reactions (≥2%) were pneumonia, febrile neutropenia, sepsis, pyrexia, and autoimmune hemolytic anemia. Deaths due to adverse reactions not related to disease progression were reported in 17 patients, most commonly (2 patients each) from septic shock and cardiopulmonary failure. Discontinuations due to adverse reactions occurred in 11% of patients. The most frequently reported adverse reactions (≥2 patients) were thrombocytopenia, autoimmune hemolytic anemia, fatigue, and multiple organ dysfunction syndrome. Dosage reductions due to adverse reactions occurred in 14% of patients. The most frequently reported adverse reactions (≥5 patients) leading to dose reductions was neutropenia. Dose interruptions due to adverse reactions occurred in 40% of patients. The most frequently reported adverse reactions (≥5 patients) leading to dose interruption were neutropenia, nausea, diarrhea, pneumonia, vomiting, febrile neutropenia, thrombocytopenia, hyperphosphatemia, pyrexia, TLS, and blood creatinine increased.

(ii). Acute Myeloid Leukemia Venetoclax in Combination with Azacitidine (in Frontline)

The safety of venetoclax (400 mg daily dose) in combination with azacitidine (n=283) versus placebo in combination with azacitidine (n=144) was evaluated in a double-blind randomized Phase 3 study in patients with newly diagnosed AML. At the time of the primary analysis, the median duration of treatment was 7.6 months (range: <0.1 to 30.7 months) in the venetoclax+azacitidine arm and 4.3 months (range: 0.1 to 24.0 months) in the placebo+azacitidine arm. The most common adverse reactions (≥15%) of any grade, reported by a higher percentage of patients (≥5% of patients) receiving venetoclax+azacitidine compared to those with placebo+azacitidine were thrombocytopenia, neutropenia, nausea, febrile neutropenia, diarrhea, vomiting, anemia, decreased appetite, peripheral edema, leukopenia, and asthenia. The most common (≥5%) Grade ≥3 AEs reported in a higher percentage of patients (by ≥2%) in the venetoclax+azacitidine arm compared to the placebo+azacitidine arm were thrombocytopenia, neutropenia, febrile neutropenia, anemia, leukopenia, and atrial fibrillation.

In the venetoclax+azacitidine arm, serious adverse reactions were reported in 83% of patients, with most frequent (≥5%) being febrile neutropenia (30%), pneumonia (23%), sepsis (16%), and hemorrhage (9%). Deaths due to AEs were reported in 23% of patients who received venetoclax in combination with azacitidine, with the most frequent (≥2%) being pneumonia (4%), sepsis (3%), and hemorrhage (2%). Adverse reactions led to venetoclax treatment discontinuations in 24% of patients, venetoclax dose reductions in 2%, and venetoclax dose interruptions in 72%. Among patients who achieved bone marrow clearance of leukemia, 53% underwent dose interruptions for ANC <500/μL. The most frequent adverse reactions (≥5%) leading to venetoclax dose interruptions in the venetoclax+azacitidine arm were febrile neutropenia (20%), neutropenia (20%), pneumonia (14%), thrombocytopenia (10%), and sepsis (8%).

Venetoclax treatment is associated with common hematological toxicities, including Grade 3 to 4 ORR of venetoclax around 40%, SD around 40%, and PD around 20%, with responses that can be significantly better in NPM1/IDH1,2 patients than in FLT3-ITD mutation carriers.

### (B). Hypomethylating Agents (HMAs): Azacitidine, Decitabine

HMAs are the current standard of care in high-risk MDS. However, only ~50% of frontline patients with MDS respond to HMAs and most responding patients eventually progress. Outcomes after HMA failure are particularly poor, with a median OS of 4 to 6 months.

### (i). HMA: Azacitidine

Azacitidine is a chemical analogue of the nucleoside cytidine, which is present in DNA and RNA. It is thought to have antineoplastic activity via two mechanisms—at low doses, by inhibiting of DNA methyltransferase, causing hypomethylation of DNA, and at high doses, by its direct cytotoxicity to abnormal hematopoietic cells in the bone marrow through its incorporation into DNA and RNA, resulting in cell death.

Adverse Reactions Described in Other Labeling Sections: anemia, neutropenia, thrombocytopenia, elevated serum creatinine, renal failure, renal tubular acidosis, hypokalemia, and hepatic coma. Most Commonly Occurring Adverse Reactions (SC or IV Route): nausea, anemia, thrombocytopenia, vomiting, pyrexia, leukopenia, diarrhea, injection site erythema, constipation, neutropenia, and ecchymosis. The most common adverse reactions by IV route also included petechiae, rigors, weakness and hypokalemia. Adverse reactions most frequently (>2%) resulting in clinical intervention (SC or IV route): discontinuation: leukopenia, thrombocytopenia, and neutropenia. Dose held: leukopenia, neutropenia, thrombocytopenia, pyrexia, pneumonia, and febrile neutropenia. Dose Reduced: leukopenia, neutropenia, and thrombocytopenia.

### (ii). HMAs: Decitabine (DACOGEN®)

Decitabine inhibits DNMT1 after incorporating into DNA during S phase. Although its mechanism of action is similar to azacitidine, the key difference lies in azacitidine's ability to incorporate into RNA, while decitabine does not.

### (C). Venetoclax, Azacitidine: Dose Modification

Note:
All dose reductions are calculated as a percentage of the starting dose.
The hematological dose modifications in this section are based on venetoclax product information.
Any abnormal blood chemistry identified before or during dose escalation for venetoclax should be corrected promptly per institutional guidelines. Risk assessment for TLS is done as per institutional guidelines and medical judgement of the treating physician. For AML patients, if the treating physician establishes that the patient is not able to tolerate venetoclax, the patient may remain on study and continue to receive G9.2-17(IgG4) with HMA only. This may be allowed in any cycle of treatment.

Tumor lysis syndrome (TLS)

| | |
|---|---|
| Blood chemistry | Withhold the next day's venetoclax dose, and if |
| suggestive of TLS | resolution occurs within 24 to 48 hours of last |
| | dose resume at the same dose. |
| Clinical TLS | Manage TLS per institutional guidelines and |
| OR | withhold venetoclax dose until recovery. |
| Blood chemistry changes | |
| for more than 48 hours | |

Hematological toxicity

| | |
|---|---|
| Bone marrow assessment | Recommend a bone marrow assessment between Days 21-28 of the |
| at the end of cycle | first cycle (C2D1 per SoA). If marrow blasts have been cleared |
| | (<5%), withhold venetoclax until commencement of Cycle 2. If |
| | persistent AML is detected, Cycle 2 should commence and C3D1 |
| | bone marrow repeated. |
| Incomplete count | If the patient has responded to treatment with <5% marrow blasts, |
| recovery after Cycle 1 | withhold the next cycle until ANC ≥0.5 × 10⁹/L and platelets ≥50 × |
| | 10⁹/L or up to 14 days after cycle completion. If neutrophils have |
| | not recovered by Day 35, commence G-CSF three times a week to |
| | assist marrow recovery. If hematological recovery has not occurred |
| | by Day 42, further treatment is at the discretion of the treating |
| | hematologist. |
| Cycle 2 onwards: | Withhold venetoclax and commence next cycle when ANC≥ 0.5 × |
| ANC <0.5 × 109/L | 10⁹/L and platelets ≥50 × 10⁹/L. Commence next cycle at the same |
| for ≥1 week (not due to | venetoclax dose level if recovery occurs within 14 days of ceasing |
| underlying disease) | venetoclax. |
| | If the time to recovery after ceasing venetoclax exceeds 14 days, |
| | reduce the duration of venetoclax by 7 days for subsequent cycles |
| | (e.g., from 28 days to 21 days to 14 days to 10 days). |
| | Venetoclax may be interrupted at the discretion of the hematologist |
| | and intermittent G-CSF commenced if ANC <0.2 × 10⁹/L until |
| | recovery to ANC ≥0.5 × 10⁹/L. |

Cycle 5 onwards:

| | |
|---|---|
| If hematological recovery | Withhold next cycle and monitor and assess counts every 7 days or |
| has not occurred 14 days | as needed. |
| after completing therapy | If hematological recovery takes >21 days after completing therapy, |
| (ANC <0.5 × 10⁹/L or | consider a reduction in the azacitidine dose according to bone |
| platelets <50 × 10⁹/L) | marrow cellularity. |

Recovery >21 days

| Bone marrow cellularity | % dose of azacitidine next cycle |
|---|---|
| 15 to 50% | 50% |
| <15% | 33% |

Hepatic impairment
Hepatic dysfunction

| | |
|---|---|
| Severe | Reduce venetoclax dose by 50% and monitor |
| (Child-Pugh C) | for signs of toxicity |

No formal studies have been conducted in patients with hepatic impairment receiving azacitidine

Renal impairment

| | |
|---|---|
| Unexplained clinically | Delay next cycle until values return to normal or baseline and reduce |
| significant elevations | azacitidine by 50% for the next cycle |
| in serum creatinine or | |
| blood urea nitrogen | |
| (BUN) | |
| Unexplained reduction | Reduce azacitidine by 50% |
| in serum bicarbonate | No dose adjustments of venetoclax necessary, however patients are at |
| levels to less than 20 | greater risk of TLS. |
| mmol/L | |
| Severe renal | Azacitidine is contraindicated |
| impairment (creatinine | However, its use has been reported in patients with CrCl <30 mL/min |
| clearance <30 | and is associated with a higher incidence of toxicity.4 |
| mL/min) | There is no data available for venetoclax in patients with severe renal |
| | impairment. |
| | Updated PK studies included in the product information for venetoclax |
| | suggest no dose modifications are required if CrCl >15 mL/min. |

For CYP3A4 or P-pg inhibitors, the following venetoclax dose reductions should be used as follows:

Dose escalation (Cycle 1 Days 1 to 3)

| | |
|---|---|
| Strong CYP3A4 inhibitor (e.g., | Modify venetoclax dose to: |
| azole antifungals) | Day 1: 10 mg |
| | Day 2: 20 mg |
| | Day 3: 50 mg |
| Moderate CYP3A4 inhibitor or | Reduce daily venetoclax dose by at |
| P-gp inhibitor | least 50% |

Post escalation (Cycle 1 Day 4 onwards)

| | |
|---|---|
| Strong CYP3A4 inhibitor (e.g., | Reduce venetoclax dose to 50 mg* |
| azole antifungals) | |
| Moderate CYP3A4 inhibitor or | Reduce venetoclax dose by at |
| P-gp inhibitor | least 50% |

*Note:
Consider an early bone marrow assessment on Day 21 of Cycle 1. Withhold venetoclax if blast clearance is documented, until commencement of Cycle 2.

Concomitant administration of investigational agents other than G9.2-17(IgG4), for any indication.
Systemic immunosuppressive treatment, including but not limited to cyclophosphamide, azathioprine, methotrexate, thalidomide, tacrolimus, cyclosporine, everolimus and sirolimus, and anti-TNF agents. However, patients are allowed to take acute, low dose systemic immunosuppressant medications (e.g., ≤10 mg/day of prednisone or equivalent).
Replacement therapy (e.g., thyroxine, insulin, physiologic corticosteroid replacement therapy [e.g., ≤10 mg/day of prednisone equivalent] for adrenal or pituitary insufficiency) is not considered a form of systemic treatment.

### (D). Venetoclax: Drug Interaction

| Venetoclax | Interaction | Clinical management |
|---|---|---|
| CYP3A4 inhibitors | Increased toxicity of | Avoid concomitant use with CYP3A |
| (e.g., aprepitant, azole | venetoclax and | inhibitors and consider alternative |
| antifungals, | increased risk of TLS | treatments. |
| clarithromycin, | at initiation and | If combination must be used, monitor |
| erythromycin, | during dose | patients closely and reduce venetoclax dose. |
| fluvoxamine, grapefruit | escalation possible | Any venetoclax dose used prior to initiating |
| juice, ritonavir, seville | due to increased | a CYP3A4 inhibitor may be resumed 2 to 3 |
| oranges etc.) | exposure | days after discontinuation of the inhibitor. |
| | | Avoid grapefruit products, seville oranges, |
| | | and starfruit during venetoclax treatment. |
| CYP3A4 inducers (e.g. | Reduced efficacy of | Avoid combination with strong or moderate |
| carbamazepine, | venetoclax due to | CYP3A4 inducers, or consider alternative |
| phenytoin, | increased clearance | agents with less CYP3A4 induction. |
| phenobarbitone, | | |
| rifampicin, St John's | | |
| wort etc.) | | |
| P-glycoprotein | Increased toxicity of | Avoid combination. |
| inhibitors (e.g., | venetoclax possible | If combination must be used monitor |
| verapamil, ciclosporin) | due to reduced | patients closely and reduce venetoclax dose. |
| | clearance | |
| P-glycoprotein | Venetoclax may alter | Avoid combination with narrow therapeutic |
| substrates (e.g., digoxin, | the absorption of the | index drugs. |
| morphine, topotecan) | substrate | If combination must be used administer at |
| | | least 6 hours before venetoclax. |
| Warfarin | Increased risk of | Monitor INR closely when used in |
| | bleeding | combination. |

### Discontinuation of Study Drug

In rare instances, it may be necessary for a patient to permanently discontinue study drug. If study drug is permanently discontinued due to reasons other than disease progression, and the patient is not being treated with other anticancer therapy(ies), the patient continues to be evaluated for disease progression for up to 2 years. See the SoA for data to be collected at the time of discontinuation of study drug and follow-up and for any further evaluations that need to be completed.

Every effort must be made by study personnel to keep patients on study treatment until one of the reasons for study treatment termination are met (disease progression, toxicity related to the study drug, withdrawal of consent). If the patient has radiographic progression but no unequivocal clinical progression and alternate treatment is not initiated, the patient may continue on study treatment. However, if patients have unequivocal clinical progression without radiographic progression, study treatment should be stopped and patients advised regarding available treatment options.

A patient may be discontinued prior to disease progression for any of the following reasons:
A DLT per definition described herein
An AE occurs/recurs outside of the DLT window that requires discontinuation of study treatment(s)
An IMAR occurs/recurs that requires discontinuation of study treatment(s)
Termination of the study by PureTech Health LLC
Intercurrent illness or medical condition that prevents further administration of treatment or may jeopardize the patient's safety if they continue on study treatment
Patient's decision
Pregnancy
Use of a non-protocol anticancer therapy
Significant deviation from protocol on the part of the patient (includes lack of compliance)
Significant protocol violation

The explanation of why the patient is discontinuing study treatment should be documented in the CRY. If the patient discontinues study treatment due to toxicity, “Dose-Limiting Toxicity” or “Adverse Event” is recorded as the primary reason for withdrawal. If a patient is prematurely discontinued from the study at any time due to an AE or SAE, the patient must be followed until resolution to Grade 2 or less, unless it is unlikely to improve because of the underlying disease.

### Concomitant Therapies

Prior and concomitant medications, including vaccines and complementary treatments/supplements, are documented for each patient at each scheduled visit (Table 7). Any medication or vaccine (including over-the-counter or prescription medicines, recreational drugs, vitamins, and/or herbal supplements) that the participant is receiving at the time of enrollment or receives during the study must be recorded along with:
Reason for use
Dates of administration including start and end dates
Dosage information including dose and frequency

### (A). Permitted Medications/Therapies

The reduction of the venetoclax dose with concomitant use of CYP3A4 inhibitors or P-gp inhibitors is summarized herein.

In addition, the following concomitant medications are allowed:
In patients on-study exceeding WBC counts of 25,000/uL, the use of hydroxyurea is permitted according to standard institutional practice for the first 2 cycles, concomitantly with G9.2-17(IgG4).
Any of the following should be treated according to standard institutional practice:
   Infusion reactions
   TLS
   CRS
The use of inhaled corticosteroids and mineralocorticoids (e.g., fludrocortisone), topical steroids, intranasal steroids, intra-articular steroids, and ophthalmic steroids.
The use of IV steroids given within the blood product transfusion protocol.
Prophylactic or therapeutic use of anticoagulants.
Vaccination for COVID-19, common flu and/or other common clinically required indications (e.g., tetanus, pneumococcus, HBV, etc.) is allowed before or during the study period. The timing and type of vaccine must be recorded.

### (B). Prohibited Medications/Therapies

The following medications are not allowed while on this study:

For G9.2-17(IgG4), closely monitor sensitive narrow therapeutic index CYP3A4 substrates such as, but not limited to, alfentanil, dihydroergotamine, ergotamine, fentanyl, pimozide, and quinidine and have doses adjusted as necessary given that G9.2-17(IgG4) can potently inhibit TGF-β and IL-10 in vitro. Reducing cytokine levels from patient baseline level may change CYP expression/activity and exposure for sensitive CYP3A4 substrates. Note: Immunosuppressant CYP3A4 substrates like tacrolimus, cyclosporine, everolimus, and sirolimus are prohibited (see below).

For venetoclax, avoid combination of venetoclax with strong or moderate CYP3A4 inducers, or consider alternative agents with less CYP3A4 induction.

### (C). Supportive Care

Patients should receive full supportive care during the study, including transfusions of blood and blood products, and treatment with antibiotics, antiemetics, antidiarrheals, and analgesics, and other care as deemed appropriate, and in accordance with institutional guidelines.

### VII. Adverse Events

### Definitions

### Adverse Events (AEs)

An AE is any event, side effect, or other untoward medical occurrence that occurs in conjunction with the use of a medicinal product in humans, whether or not considered to have a causal relationship to this treatment. An AE can, therefore, be any unfavorable and unintended sign (that could include a clinically significant abnormal laboratory finding), symptom, or disease temporally associated with the use of a medicinal product, whether or not considered related to the medicinal product.

Events meeting the definition of an AE include:
Exacerbation of a chronic or intermittent pre-existing condition including either an increase in frequency and/or intensity of the condition
New conditions detected or diagnosed after study drug administration that occur during the reporting periods, even though it may have been present prior to the start of the study
Signs, symptoms, or the clinical sequelae of a suspected interaction
Signs, symptoms, or the clinical sequelae of a suspected overdose of either study drug or concomitant medications (overdose per se will not be reported as an AE/SAE)

Events that do not meet the definition of an AE include:
Medical or surgical procedure (e.g., endoscopy, appendectomy); the condition that leads to the procedure should be reported as an AE if it meets the criteria of an AE
Situations where an untoward medical occurrence did not occur (e.g., social and/or convenience admission to a hospital)
Anticipated day-to-day fluctuations of pre-existing disease(s) or condition(s) present or detected at the start of the study that do not worsen

If there is evidence of an AE through report or observation, the following information should be further evaluated and recorded:
Time of onset and resolution
Severity
Causality/relation to study treatment
Action taken regarding study drug
Outcome

When an AE occurs, all documentation (e.g., progress notes, laboratory results, diagnostic reports) relevant to the event will be reviewed.

### Serious Adverse Events

An SAE is any event that meets any of the following criteria:
Results in death.
Is life-threatening.
Requires inpatient hospitalization or prolongation of existing hospitalization.
Results in persistent or significant disability/incapacity.
Is a congenital anomaly/birth defect.
Is an important medical event that may not result in death, be life-threatening, or require hospitalization. The event is considered an SAE when, based upon appropriate medical and scientific judgment, the event may jeopardize the patient and may require medical or surgical intervention to prevent one of the outcomes listed above. Examples of such events include: intensive treatment in an emergency room or at home for allergic bronchospasm, blood dyscrasias or convulsions that do not result in inpatient hospitalization, or the development of drug dependency or drug abuse.

### Definition of Terms

Life-threatening: an AE is life-threatening if the patient was at immediate risk of death from the event as it occurred; i.e., it does not include a reaction that, if it had occurred in a more severe form, might have caused death. For example, drug-induced hepatitis that resolved without evidence of hepatic failure would not be considered life-threatening even though drug-induced hepatitis can be fatal. It does not include an AE or suspected adverse reaction that, had it occurred in a more severe form, might have caused death.
Hospitalization: AEs requiring hospitalization should be considered SAEs. Hospitalization for elective surgery, or for procedures planned prior to the patient providing informed consent, or routine clinical procedures that are not the result of an AE (e.g., elective surgery for a pre-existing condition that has not worsened, surgical insertion of central line) need not be considered AEs or SAEs. However, the medical condition for which the procedure was performed should be reported if it meets the definition of an AE. For example, an acute appendicitis that begins during the AE reporting period should be reported as the AE, and the resulting appendectomy should be recorded as treatment of the AE. If anything untoward is reported during the procedure, that occurrence must be reported as an AE, either “serious” or “nonserious” according to the usual criteria.
In general, hospitalization signifies that the patient has been detained (usually involving at least an overnight stay) at the hospital or emergency ward for observation and/or treatment that would not have been appropriate in the physician's office or outpatient setting. Exceptions include: rehabilitation facilities, hospice facilities, respite care (e.g., caregiver relief), skilled nursing facilities, nursing homes; all of which do not constitute as hospitalization. When in doubt as to whether hospitalization occurred or was necessary, the AE should be considered serious.
Disability/incapacity: an AE is incapacitating or disabling if the experience results in a substantial and/or permanent disruption of the patient's ability to carry out normal life functions.

### Suspected Unexpected Serious Adverse Reactions (SUSARs)

A SUSAR is an AE that is both unexpected (i.e., not listed in the Reference Safety Information for the investigational agent) and meets the definition of a serious adverse drug reaction, the specificity or severity of which is not consistent with those noted in the Reference Safety Information (i.e., the IB for an investigational agent).

### (i). Immune-Mediated Adverse Reactions

G9.2-17(IgG4) is considered an immunomodulatory monoclonal antibody and as such, in the early phase of clinical development, may be classified within a broader class of immune checkpoint inhibitors (CPIs). IMARs, which may be severe or fatal, can occur in any organ system or tissue in patients receiving immune active therapy.

### (ii). Infusion-Related Reactions (IRRs)

IRRs (to include cytokine release syndrome [CRS]) are a known side effect of mAb therapy and a potential expected side effect of G9.2-17(IgG4) treatment. Symptoms can include rash, fever, rigors, pain, bronchospasm, and hypotension. This type of reaction is anticipated in patients receiving monoclonal antibodies.

If an IRR occurs during or after any infusion, all supportive measures consistent with optimal patient care should be administered according to institutional standards and in adherence with the implemented clinical study protocols. In G9.2-17(IgG4) trials, patients may receive premedication as described in the clinical study protocols prior to G9.2-17(IgG4) infusions and are closely monitored for signs of acute infusion reaction. In the present study, G9.2-17(IgG4)-2022-02, IRRs are considered to be adverse events of special interest (AESIs).

(iii). Tumor Lysis Syndrome

G9.2-17(IgG4) induces apoptosis of AML cells by blocking gal-9 and not by an Fc-mediated G9.2-17(IgG4) function. As such, G9.2-17(IgG4) could pose a risk for tumor lysis syndrome (TLS). No TLS-like side effects have been observed in mouse models engrafted with AML cells treated with G9.2-17(IgG4). In the present study, G9.2-17(IgG4)-2022-02, the following precautions are implemented to monitor and treat potential TLS:
In order to provide for adequate monitoring of laboratory parameters associated with TLS during the first week of G9.2-17(IgG4) treatment, in addition to the scheduled blood draw on Cycle 1 Day 1, a second blood draw is collected on Cycle 1 Day 2.
Patients may be risk assessed for TLS prior to each G9.2-17(IgG4) infusion and, where clinically indicated (e.g., TLS experienced with prior therapy regimens), treated according to institutional standard of care, such as with adequate hydration and uricostatics prior to the start of the G9.2-17(IgG4) infusion.
Where a patient has a high disease burden and is at greater risk of TLS, it may also be appropriate to keep/admit them for additional monitoring following G9.2-17(IgG4) administration.

### (iv). Anti-Drug Antibodies (ADAs)

Development of anti-G9.2-17(IgG4) antibodies (ADAs) is possible, and the potential risks associated with ADAs are unknown. In the present study, patients are monitored and treated according to standard institutional practice.

### Clinical Laboratory Abnormalities and Other Abnormal Assessments

Abnormal laboratory findings (e.g., clinical chemistry, hematology, and urinalysis) or other abnormal assessments (e.g., ECGs or vital signs) as clinically significant is recorded as AEs and SAEs if they meet the definition of an AE or SAE. Clinically significant abnormal laboratory findings or other abnormal assessments that are detected during the study or are present at screening and significantly worsen following the start of the study is reported as AEs or SAEs. However, clinically significant abnormal laboratory findings or other abnormal assessments that are associated with the disease being studied, unless considered as more severe than expected for the patient's condition, or that are present or detected at the start of the study and do not worsen, are reported as AEs or SAEs.

Laboratory measurements that deviate clinically significantly from previous measurements may be repeated. If warranted, additional or more frequent testing than is specified in the protocol should be done to provide adequate documentation of AEs and the resolution of AEs. It will be decided whether an abnormal laboratory finding, or other abnormal assessment is clinically significant.

### Adverse Events of Special Interest

An AESI is an AE of scientific and medical concern specific to the product or program, which requires additional monitoring. Such an event might warrant further investigation to better characterize and understand it.

For this study the following events is considered AESIs if the event has severity of Grade 3 or higher:
IMARs
IRRs
CRS
TLS

AESIs must be reported to PureTech Health Pharmacovigilance department or its designee within the same timeframe as that which applies for SAEs. Dose modification instructions for these AESIs are described herein and found in Tables 8 and 9 (IMAR management and LYT dose mods).

### Assessment of Adverse Events

### Severity

All AEs are graded by severity using CTCAE version 5.0.

If an AE is not listed in the CTCAE criteria, a corresponding grading is to be performed based on his/her best medical judgment as follows:
Mild (Grade 1): asymptomatic or mild symptoms; clinical or diagnostic observations only; intervention not indicated
Moderate (Grade 2): minimal, local, or noninvasive intervention indicated; limited age appropriate instrumental activities of daily living (ADL)
Severe (Grade 3): medically significant but not immediately life-threatening; hospitalization or prolongation of hospitalization indicated; disabling; or limiting self care ADL
Life-threatening (Grade 4): life-threatening consequences; urgent intervention indicated
Death (Grade 5): death related to an AE

Note: The terms “severe” and “serious” are not synonymous. Severity is a measure of intensity (as characterized above) whereas seriousness as defined above, defines the requirements for reporting obligations to applicable regulatory authorities.

### Causality Assessment

The causal relationship between and the occurrence of all AEs should be assessed using best clinical judgement and taking into consideration the natural history of the underlying disease, concomitant therapies, biological plausibility, the mechanism of action of the study drug, and the temporal relationship between the event and the administration of study drug.

Each AE should be assessed based on all available information at the time of reporting. Causality assessments may be modified in the context of additional information or details pertaining to the event, after the initial causality assessment is made. The relationship of the AE to the study treatment is assessed following the definitions below:
Unrelated: any event that does not follow a reasonable temporal sequence from administration of study drug AND that is likely to have been produced by the patient's clinical state or other modes of therapy administered to the patient.
Unlikely Related: any event that does not follow a reasonable temporal sequence from administration of study drug OR that is likely to have been produced by the patient's clinical state or other modes of therapy administered to the patient.
Possibly Related: any reaction that follows a reasonable temporal sequence from administration of study drug OR that follows a known response pattern to the suspected drug AND that could not be reasonably explained by the known characteristics of the patient's clinical state or other modes of therapy administered to the patient.
Related: any reaction that follows a reasonable temporal sequence from administration of study drug AND that follows a known response pattern to the suspected drug AND that recurs with re-challenge, AND/OR is improved by stopping the drug or reducing the dose.

In addition, if an SAE is associated with study procedures, this causal relationship must be recorded in the source documents and CRF, as appropriate, and report such an assessment in accordance with the SAE reporting requirements, if applicable.

### Documenting and Reporting Adverse Events

All observed or volunteered AEs are recorded regardless of treatment group or causal relationship to study drug is recorded on the AE page(s) of the CRF. AEs are coded using the Medical Dictionary for Regulatory Activities (MedDRA) coding system and all AEs are graded according to the National Cancer Institute Common Terminology Criteria for Adverse Events, version 5.0 (NCI-CTCAE).

For all AEs, information must be pursued and obtained that is adequate to both determine the outcome of the AE and to assess whether it meets the criteria for classification as an SAE requiring immediate notification. For all AEs, sufficient information should be obtained to determine the causality of the AE. For AEs with a causal relationship to the IP, follow-up is required until the event resolves or stabilizes at a level acceptable.

Events meeting the criteria and definition of an AE or SAE, as provided in this protocol, are detected and documented. When there is a safety evaluation during the study, AEs and SAEs, as detailed in this section of the protocol, are detected, documented, and reported.

The CRF should include the following information: start and stop dates, CTCAE v. 5.0 grading, relationship to study medication, action taken, and outcome. AEs should be recorded from the time of signing informed consent to 30 days following the last dose of study medication regardless of the causal relationship to the study medication. AEs considered related or possibly related to study medication should be recorded at any time regardless of when they occurred.

Possible relationship between the AE and any of the study medications are assessed as well as any concomitant medications according to the criteria presented herein

### Adverse Event Outcomes

AE outcomes are recorded using the following categories:
Recovered/resolved
Recovering/resolving
Not recovered/not resolved
Recovered/resolved with sequelae
Fatal
Unknown

### Reporting of Serious Adverse Events

All SAEs, whether or not considered related to study drug, must be reported when the site becomes aware of the event(s) by completing the SAE form electronically in the electronic data capture (EDC) system for the study within 24 hours of first awareness. Any AESI or SAEs assessed as related to study participation (e.g., study intervention, protocol-mandated procedures, invasive tests, or change in therapy) are recorded from the time a patient consents to participate in the study.

For any other experience or condition that meets the definition of an SAE, recording of the event must begin from Day 1 of study treatment and continue through the 30 day follow-up period after treatment is discontinued.

In the event EDC is unavailable, the paper SAE form must be completed. As soon as EDC is available, the event from the paper SAE report form, including any updated information, must be entered into EDC.

SAEs related to G9.2-17(IgG4) are promptly reported to the FDA and other applicable regulatory agencies in accordance with Title 21 of the Code of Federal Regulations (CFR), Part 312.32 (21 CFR 312.32) and local regulatory requirements.

### Adverse Event and Serious Adverse Event Follow-Up

After the initial AE/SAE report, each participant should be proactively followed at subsequent visits/contacts. All SAEs are followed until resolution, stabilization, the event is otherwise explained, or the participant is lost to follow-up (as defined herein).

The conduct of supplemental evaluations to elucidate as fully as possible the nature and/or causality of any event may be performed or arranged.

### Pregnancy

If a female patient becomes pregnant while receiving or within 180 days of discontinuing study treatment, OR the female partner of a male patient becomes pregnant while he is receiving or within 180 days of discontinuing study treatment, a pregnancy report form must be completed to facilitate outcomes follow-up. While pregnancy itself is not considered an AE, any pregnancy complication or elective termination of a pregnancy for medical reasons are recorded as an AE. An abortion whether therapeutic or spontaneous must be reported as an SAE.

For pregnancies taken to term, details on the status of the mother and child are recorded, with follow-up generally no longer than 6 to 8 weeks after the delivery date. Any congenital anomaly in a child born to a patient exposed to study treatment must be reported as a SAE.

Details of all pregnancies in female participants and, if indicated, female partners of male participants are collected after the start of study intervention and until 180 days after last treatment.

If a pregnancy is reported, pregnancy information is recorded on the appropriate form within 24 hours (one calendar day) of learning of the [female participant or female partner of male participant (after obtaining the necessary signed informed consent from the female partner)]pregnancy.

While pregnancy itself is not considered to be an AE or SAE, any pregnancy complication or elective termination of a pregnancy for medical reasons is reported as an AE or SAE.

Abnormal pregnancy outcomes (e.g., spontaneous abortion, fetal death, stillbirth, congenital anomalies, ectopic pregnancy) are considered SAEs and are reported as such.

The participant/pregnant female partner is followed to determine the outcome of the pregnancy. follow-up information is collected on the participant/pregnant female partner and the neonate.

Any female participant who becomes pregnant while participating in the study discontinues the study drug or is withdrawn from the study.

Disease-Related Events and/or Disease-Related Outcomes Qualifying as SAEs

It is anticipated that some patients will experience disease progression before study discontinuation, and the disease progression may result in hospitalization or death. Where possible, the specific event defining the disease progression should be reported, such as pleural effusion, intestinal obstruction or cardiac failure. Only if it is not possible to discern the specific event leading to disease progression should “disease progression” be reported as the event. In addition, SAEs that are assessed as possibly or related to study drug and occurring greater than 30 days after the last dose of study drug also requires reporting to the PureTech Health Pharmacovigilance department or its designee within 24 hours, or when the site becomes aware of the event.

### VIII. Statistics

### General Procedures

A database lock and primary analysis is performed after the last patient has had their primary endpoint event. A final study analysis is performed after study completion. All analyses are descriptive. Unless otherwise stated, descriptive statistics are presented by dose and overall.

Unless otherwise specified, all continuous endpoints are summarized using descriptive statistics, which includes the number of patients (n), mean, standard deviation, median, minimum, and maximum. All categorical endpoints are summarized using frequencies and percentages, with 95% CI. The baseline measurement is the last value on or before the date of first study treatment. Survival curves for time-to-event endpoints, e.g., EFS and OS for all cohorts combined, is generated by the method of Kaplan-Meier from the day treatment was initiated. Waterfall and Swimmers plots are used to graphically present the ORR and DoR for all patients in all cohorts combined. All statistical analyses are performed using SAS® version 9.4 (SAS, Cary, NC) or higher.

### Protocol Deviations

In general, a protocol deviation is an inadvertent excursion to or non-compliance with the IRB approved protocol. Any changes to the protocol should not be implemented unless they are required to eliminate an immediate hazard to the patient. Deviations are captured electronically. Whether a protocol deviation is major or minor is determined prior to database lock.

### Analysis Populations

The intent-to-treat (ITT) population is defined as those patients who received at least one dose of G9.2-17(IgG4), unless otherwise specified. The efficacy analyses are performed for the ITT. Patient disposition is performed for the ITT.

The Efficacy population is defined as all patients in the ITT and having at least one measurable overall response at 1 month. This population is used for a sensitivity analysis of efficacy endpoint(s) if appropriate.

The PP population includes any patient who received at least one full cycle of G9.2-17(IgG4) and without major protocol deviations.

The safety population (SAF) includes all patients who receive at least one dose of G9.2-17(IgG4). The safety analyses is performed for the SAF.

The PK population includes any patients who received at least one dose of G9.2-17(IgG4) and have at least one evaluable post-dose concentration.

The PD population is defined as those patients who have received at least one full cycle of G9.2-17(IgG4) treatment and who have at least one post-treatment PD assessment along with a baseline PD assessment.

### Sample Size

This dose escalation study establishes either the MTD and/or the minimum safe and effective biological dose to determine the RP2D dose or doses for G9.2-17(IgG4) using a 4+2 algorithm-based dose-escalation design (Lin et al., Biostatistics, 2001, 2, 203-15; Wheeler et al., PLoS One, 2016, 11, e0159026).

With the potential inclusion of patient backfill, it is anticipated that a total sample size of up to 90 patients are enrolled into the study in Part 1 and Part 2. Part 1 up to 50 patients (5 cohorts), Part 2 up to 40 patients (4 cohorts).

### Statistical Methods

### Primary Endpoints

The primary endpoints are:

Evaluation of safety parameters including AEs, vital sign measurements, clinical safety laboratory tests, 12-lead ECG, echocardiography/cardiac ultrasound (ECHO), physical examinations.

### (i) Evaluation of First 4 Patients for Each Cohort for Dose Escalation

DLT 0/4: Progress to next dose level.

DLT ¼: Evaluate 2 additional patients at the same dose level.

DLT ⅙: Progress to next dose level.

If ≥2 patients in a dose level of 4 to 6 patients experiences DLT, then the (MTD) will have been exceeded.

### (ii) Determination of RP2D

The RP2D is determined based on DLTs, PK, PD, and additional safety data observed in each dose level and any other factors that may need to be considered.

### Evaluation of DLTs During Cycle 1

Refer to the safety section.

(iii) Primary Endpoint Analysis: Safety

All safety analyses are made on the SAF unless otherwise specified.

a. Adverse Events

Treatment-emergent adverse events (TEAEs) are defined as events that occur on or after the first dose of study medication. The MedDRA coding dictionary is used for the coding of AEs. TEAEs, serious or CTCAE Grade 3 or Grade 4 TEAEs, and TEAEs related to treatment are summarized overall and by system organ class (SOC) and preferred term (PT) by treatment group. These summarize the number of events and the number and percent of patients with a given event. In addition, the number and percent of patients with TEAEs is provided by maximum severity. A summary of all TEAEs by SOC and PT occurring in ≥5% of patients in either treatment group is provided.

Number and frequencies of patients with events as well as number of events is given by SOC and by PT within each SOC for the following:
Overview of AEs and TEAEs
All AEs by SOC and PT
All TEAEs by SOC and PT
Serious TEAEs by SOC and PT
Most frequent TEAEs by SOC and PT, defined as occurring in at least 5% of patients in any dose cohort
TEAEs of CTCAE grade 3 or higher by SOC and PT
TEAEs considered at least possibly related to study drug by SOC and PT
Serious TEAEs considered related to study drug by SOC and PT
TEAEs by maximum severity and by SOC and PT
DLTs by SOC and PT
TEAEs leading to study discontinuation of study drug by SOC and PT
IRR by SOC and PT
IMARs by SOC and PT

Patient listings are provided for patients with AEs, TEAEs, SAEs and for all patients with AEs leading to discontinuation of study drug, AEs leading to discontinuation of study, and DLTs.

b. Laboratory Assessments

All laboratory-based data is presented as listings of all values as well as of abnormal results judged to be clinically significant, which are also reported as AEs. Numeric summaries of all observed findings and changes from baseline screening laboratory evaluations are provided by visit and treatment group, including chemistry, hematology, and urinalysis results. No inferential comparisons are planned.

c. Vital Signs

Numeric summaries of all observed findings and changes from baseline screening vital signs are provided by time point and treatment group, including blood pressure, heart rate, respiratory rate, and temperature. No inferential analyses are planned for vital signs.

d. ECGs, ECHO/MUGA, and Physical Examination

Physical examination data and changes are presented as listings. ECG results are presented as listings and summarized by treatment group and visit, based on incidence of clinically significant abnormalities. No inferential comparisons across treatment groups are planned.

e. Primary Endpoint Analysis: DLTs, RP2D

DLTs are summarized.

### Secondary Endpoints

### (i) Preliminary Efficacy (Disease Response)

Disease response assessed are summarized descriptively for the ITT and PP populations. A sensitivity analysis is performed for the EFF population if appropriate.

For efficacy endpoints, see Section 3.2.

### (ii) Pharmacokinetics

The PK endpoints are:
Evaluation of PK parameters of G9.2-17(IgG4) (including but not limited to, area under the curve from time zero until 168 h [AUC₀₋₁₆₈ₕ], maximum observed serum concentration [Cₘₐₓ], time to reach Cₘₐₓ [Tₘₐₓ], trough serum concentration [C_{trough}])

PK parameters are summarized descriptively for the PK population. Details on PK analysis are described in a separate analysis plan. Population PK analysis may be performed and the relationships between PK, PD, clinical labs, safety, and/or efficacy observations may be explored.

### Exploratory Endpoint(s)

The exploratory PD immunological and molecular changes in peripheral blood and bone marrow endpoints are:
Multiplexing of serum/plasma cytokines
Mass cytometry whole blood immune profiling
Gene array from whole blood
   PD endpoints are summarized descriptively for the PD population.
   The exploratory immunogenicity endpoints are:
Evaluation of ADA

Immunogenicity is summarized descriptively for the SAF.

### Results

Results from the above-described clinical trial show that single agent G9.2-17(IgG4) in relapsed refractory AML/high risk MDS was well tolerated at all dose levels from 2-16 mg/kg dosed weekly with no dose limiting toxicities or treatment related SAEs. Same was seen in single agent G9.2-17(IgG4) in relapsed refractory AML/high risk MDSat dose levels of G9.2-17(IgG4) at 4-12 mg/kg. The combination of G9.2-17(IgG4) with venetoclax and HMA (azacitidine or decitabine) did not enhance side effects associated with venetoclax or the HMA. The safety profile of the G9.2-17(IgG4) single agent cohorts and the combination cohorts is provided in Table 12 below.

TABLE 12
Safety Profile

| | Single Agent | Combination |
|---|---|---|
| | (N = 39) | (N = 62) |
| Parameter | N (%) | N (%) |
| Patients with any TEAEs | 38 (97.4) | 62 (100) |
| Serious TEAEs | 26 (66.7) | 41 (66.1) |
| TEAEs related to G9.2- | 19 (48.7) | 29 (46.8) |
| 17(IgG4) | | |
| Serious TEAEs related to G9.2- | 0 | 0 |
| 17(IgG4) | | |
| Infusion-Related Reactions to | 0 | 0 |
| G9.2-17(IgG4) | | |
| Immune-Mediated Adverse | 0 | 0 |
| Reactions | | |
| TEAEs leading to Dose- | 0 | 0 |
| Limiting Toxicities | | |
| TEAEs Leading to Study | 0 | 0 |
| Treatment Discontinuation | | |
| ≥Grade 3 TEAEs Related to | 1 (2.6)¹ | 6 (9.7) |
| G9.2-17(IgG4) | | |
| Patients with TEAEs Leading | 0 | 7 (11.3) |
| to Death | | |
| Patients with TEAEs Leading | 0 | 0 |
| to Death Related to G9.2- | | |
| 17(IgG4) | | |

* TEAEs = treatment-emergent adverse event
¹Grade 3 asymptomatic troponinemia, reversable and transient

Adverse events observed in the single agent cohorts include hypokalemia, fatigue, febrile neutropenia, blood creatinine increase, cough, decreased appetite, hyponatremia, nausea, diarrhea, ALT increase, hypoalbuminemia, and edema peripheral. Adverse events observed in the combination cohorts include hypokalemia, fatigue, anemia, febrile neutropenia, nausea, thrombocytopenia, hyperbilirubinemia, constipation, diarrhea, dyspnea, neutropenia, decreased appetite, AST increase, pneumonia, and vomiting. These adverse events are not related to the anti-Gal9 antibody.

Single agent G9.2-17(IgG4) in relapsed refractory AML/high risk MDS showed clinical efficacy, for example, at dose levels of 7.5 mg/kg and above and produced partial responses, hematological improvement, transfusion independence, reduction of blasts in bone marrow and periphery, as well as increase in % donor chimerism in a patient treated with singe agent G9.2-17(IgG4) post transplant. G9.2-17(IgG4) as a single agent also showed long time on treatment, indicating that G9.2-17(IgG4) can prolong survival in relapsed and/or refractory AML/MDS patients.

The combination of G9.2-17(IgG4), venetoclax, and HMA (azacitidine or decitabine) in AML patients and the combination of G9.2-17(IgG4) and HMA (azacitidine or decitabine) in MDS patients resulted in complete response with minimal residual disease (CR MRD⁺), as well as long term stable disease.

The combined treatment efficacy (AML+MDS) is provided in Table 13A (single agent cohorts) and Table 13B (combination cohorts) below.

TABLE 13A
Combined Treatment Efficacy in Single Agent Cohorts

| G9.2- | | | | | |
|---|---|---|---|---|---|
| 17(IgG4) | 2 | 4 | | | |
| Dose | mg/kg | mg/kg | 7.5 mg/kg | 12 mg/kg | 16 mg/kg |
| Combined | SD: | SD: | cCR: 14.3% | ORR: 25% | ORR: 10% |
| Efficacy | 50% | 50% | ORR: 28.6% | SD: 25% | SD: 60% |
| | | | SD: 57.1% | DCR: 50% | DCR: 70% |
| | | | DCR: 85.7% | | |

* SD = stable disease;
ORR = overall response rate;
cCR = combined complete response (CRMRD+, CRMRD− and CRi for AML, and CRMRD+, CRMRD−, mCR, marrow CR for MDS; CRi = CR with incomplete hematological recovery);
DCR = disease control rate

TABLE 13B
Combined Treatment Efficacy in Single Agent Cohorts

| G9.2- | | | |
|---|---|---|---|
| 17(IgG4) | | | |
| Dose | 4 mg/kg | 7.5 mg/kg | 12 mg/kg |
| Combined | SD: 75% | cCR: 20% | cCR: 32.4% |
| Efficacy | | ORR: 30% | ORR: 43.2% |
| | | SD: 40% | SD: 45.9% |
| | | DCR: 70% | DCR: 89.1% |

* SD = stable disease;
ORR = overall response rate;
cCR = combined complete response (CRMRD+, CRMRD− and CRi for AML, and CRMRD+, CRMRD−, mCR, marrow CR for MDS; CRi = CR with incomplete hematological recovery);
DCR = disease control rate

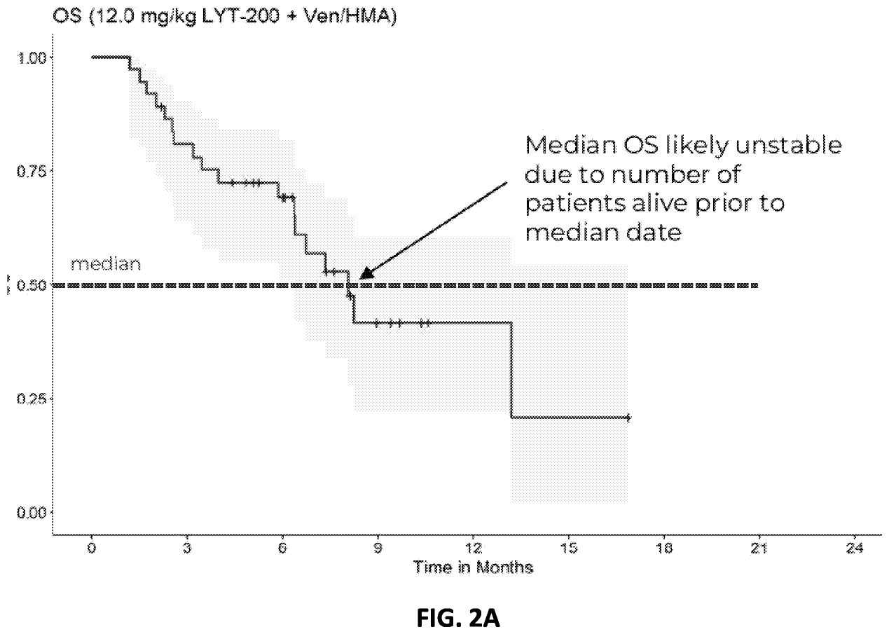

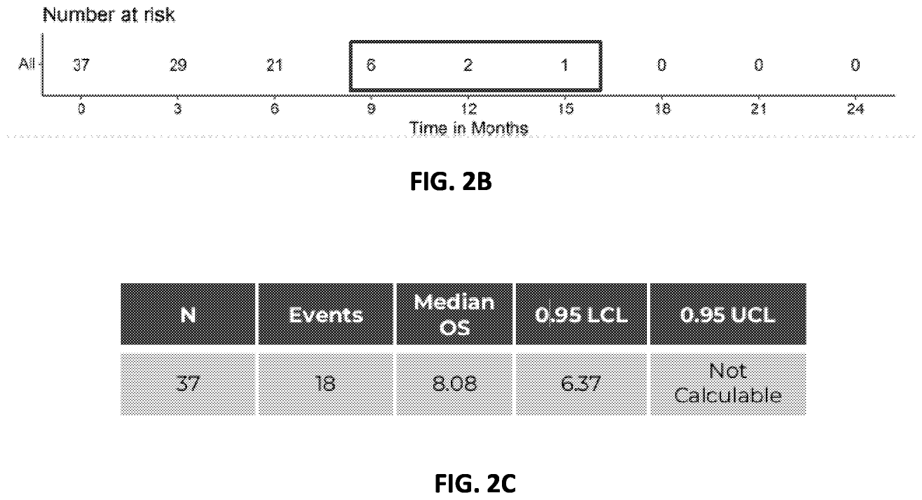

See also FIGS. 2A-2C for results from the 12 mg/kg combination cohort.

### AML Results

All resistant and/or refractory AML patients subjected to the treatments disclosed herein had at least 2 median prior lines of therapy. 84.6% of the patients were pre-treated with venetoclas and hypomethylating agents (4 patients were naïve to venetoclax and 4 patients were naïve to hypomethylating agents). Some of the patients carry mutations associated with venetoclax resistance (such patients are responders to the treatment disclosed herein). All patients have intermediate and adverse cytogenetics.

As shown in Table 14 below, the 12 mg/kg combination cohort met the KOL success criteria.

TABLE 14
Treatment Outcome of 12 mg/kg Combination
Cohort Meets KOL Success Criteria (N = 26)

| | | AML Only | KOL-defined |
|---|---|---|---|
| | | N (%) | Success |
| Efficacy | CR + CRi | 8 (30.8) | 20-30% |
| | | 3 CR MRD+; 1 CR | |
| | | MRD−, and 4 CRi | |
| | PR | 2 (7.7) | |
| | MLFS | 1 (3.8) | |
| | ORR | 11 (42.3) | ≥30% |
| | SD | 12 (46.2) | |
| | Conversion to Transplant | 5 (19.2) | |
| | Rate | | |
| Survival | mOS (data not mature as | 8.2 months | 6-9 months |
| | study ended with many | | |
| | patients still alive and no | | |
| | survival data are | | |
| | collected). | | |

* CR = complete response; CRi = complete response with incomplete hematological recovery; PR = partial response; MLFS = morphologic leukemia-free state; ORR = overall response rate; SD = stable disease; mOS= median overall survival.

It is estimated that the expected outcomes in venetoclax-naïve AML patients are 30-40% of CR+CRi, which would drop to 10-15% in rechallenge patients. The expected outcomes in hypomethylating agent rechallenge AML patients are 11% CR and 5.3 CRi (total 16.3%). Given the percentage of AML patients with prior venetoclax and/or hypomethylating agent treatments, the results observed in the combination cohort indicate that G9.2-17(IgG4) has a contribution of effect in AML (resistant and/or refractory) patients.

### MDS Results

MDS is characterized by accumulation of mutations and genetic rearrangements that cause ineffective hematopoiesis and occurrence of myeloid blasts. MDS can transform into AML, which is characterized by marrow blasts often significantly higher than 10% that deplete and displace normal cells in the marrow. MDS is a devastating disease with poor survival outcomes.

Among the high-risk MDS patients subjected to the treatment disclosed herein, all patients had at least 3 median prior lines of therapy and were pretreated with hypomethylating agents (HMA); 45% of the patients were pretreated with venetoclax as well, and all have high-risk cytogenetics.

As shown in Table 15 below, the 12 mg/kg combination cohort met the KOL success criteria. KOL stands for “Key Opinion Leader.”

TABLE 15
Treatment Outcome of Combination Cohort
Meets KOL Success Criteria (N = 11)

| | | AML Only | KOL-defined |
|---|---|---|---|
| | | N (%) | Success |
| Efficacy | CR + CRi | 3 (27.3) | 7% |
| | | 1 CR MRD+; 2 CR | |
| | | MRD− | |
| | Marrow CR¹ | 1 (9) | |
| | PR | 1 (9) | |
| | CR + PR | 4 (36.3) | |
| | ORR | 5 (45.5) | ≥30% |
| | SD | 2 (18) | |
| | Conversion to Transplant | 5 (19.2) | |
| | Rate | | |
| Survival | mOS (data not mature | 6.4 months | 6-9 months |
| | due to the timing of when | | |
| | the study was ended) | | |

* CR = complete response;
CRi = CR with incomplete hematological recovery;
ORR = overall response rate;
SD = stable disease;
mOS = median overall survival
¹2025 FDA draft MDS 2026 advise not to count marrow CR into ORR but to focus on CR or CR + PR

Prior reports indicate that HMA has little contribution of effects in MDS patients. Braun et al., Blood (2013) 122(21): 2796; Duong et al., Leuk Lymphoma (2015) 56(6): 1718-1722; Apuri et al., Clin. Lymphoma, Myeloma, Leuk. (2017) 17(4): 211-214; and Borthakur et al., Leuk Lymphoma (2008) 49(4): 690-695. Rigorous statistical analysis, including Fisher's exact test and Bayesian model for binomial response, shows that the treatment efficacy observed in the MDS patients is unlikely due to random chance. Tipping point analysis indicates high probability that anti-galectin-9 antibodies such as G9.2-17(IgG4) would show significant improvement over HMA treatment in MDS patients (specifically high risk MDS patients).

Preliminary Data Suggesting Pathways Affected by Anti-Gal9 Antibody-Containing Treatment Regimen, which May Work to Address Venetoclax and/or Hypomethylating Agent Resistance

Table 16 below lists genes that are (i) up-regulated in responders to G9.2-17(IgG4) and down-regulated in non-responders, and (ii) down-regulated in responders to G9.2-17(IgG4) and up-regulated in non-responders, and the biological pathways associated with such genes.

TABLE 16
Differentiated Expression of Genes
in Responders and Non-Responders

| | | | | |
|---|---|---|---|---|
| | | | P* | |
| Genes | Log2FC | P | adj | Biological Pathway |

Increased in responders/decreased in non-responders

| | | | | |
|---|---|---|---|---|
| CD3G | 0.59 | 0.001 | 0.038 | T-cell activation and signal |
| | | | | transduction |
| CD4 | 0.56 | 0.000 | 0.014 | MHC II-restricted antigen |
| | | | | recognition |
| CTSS | 0.56 | 0.005 | 0.096 | MHC Class II antigen processing |
| CD48 | 0.55 | 0.000 | 0.014 | T cell, NK cell activation |
| ENTPD1 | 0.55 | 0.001 | 0.034 | Immune suppression via adenosine |
| (CD38) | | | | production |
| LILRA1 | 0.55 | 0.003 | 0.084 | Innate immunity |
| DPP4 | 0.54 | 0.003 | 0.084 | T-cell activation and cytokine |
| (CD26) | | | | processing |
| NOD2 | 0.53 | 0.000 | 0.014 | Innate immunity |
| CD3D | 0.52 | 0.004 | 0.092 | T cell development and signaling |
| KLRB1 | 0.52 | 0.003 | 0.084 | Receptor expressed on NK cells, |
| | | | | MAIT cells, and memory cells |
| LY96 | 0.50 | 0.004 | 0.034 | TLR4 co-receptor, innate immunity |
| CD3E | 0.50 | 0.004 | 0.094 | T-cell activation |
| GIMAP4 | 0.49 | 0.004 | 0.094 | T-cell survival, differentiation, and |
| | | | | pro-apoptotic |
| SOCS1 | 0.49 | 0.000 | 0.030 | Suppressor of cytokine and |
| | | | | JAK-STAT signaling |
| TNFAIP3 | 0.48 | 0.004 | 0.092 | Negative regulator of NFκb, |
| | | | | inflammation and excessive |
| | | | | immune activation |
| TNFSF8 | 0.46 | 0.003 | 0.084 | T-cell activation and proliferation |
| CD27 | 0.45 | 0.003 | 0.086 | T-cell survival and memory |
| | | | | formaiton |
| CCR4 | 0.45 | 0.003 | 0.086 | Chemokine receptor involved in |
| | | | | T-cell trafficking |
| CASP1 | 0.44 | 0.002 | 0.084 | Activator of inflammatory |
| | | | | cytokines and cell death |
| TNFSF12 | 0.36 | 0.003 | 0.086 | Inflammation, tissue remodeling, |
| | | | | angiogenesis, and pro-apoptotic |
| IL12RA | 0.34 | 0.002 | 0.084 | Marker of activated T cells and |
| | | | | regulatory T cells |
| GZMB | 0.33 | 0.001 | 0.055 | Cytotoxic protease used by |
| | | | | cytotoxic T cells and NK cells |
| UBE2T | 0.24 | 0.004 | 0.093 | Anti-DNA damage cycle |

Increased in non-responders/decreased in responders

| | | | | |
|---|---|---|---|---|
| NECTIN2 | −0.63 | 0.002 | 0.084 | Cell adhesion, immune ligand |
| (CD112) | | | | modulating T-cell and NK cell |
| | | | | responses |
| FAM30A | −0.63 | 0.003 | 0.086 | Immune-regulatory IncRNA |
| GLS | −0.55 | 0.001 | 0.034 | Metabolism |
| ITGAV | −0.49 | <0.001 | 0.027 | Migration and angiogenesis |
| HELLS | −0.42 | 0.003 | 0.084 | Chromatin remodeling and |
| | | | | genome stability |
| DNMT1 | −0.39 | <0.001 | 0.017 | Epigenetic regulator |
| NOTCH1 | −0.39 | <0.001 | 0.027 | Signaling receptor controlling |
| | | | | cell fate, especially |
| | | | | immune and stem cells |
| HRAS | −0.39 | <0.001 | 0.027 | Cell proliferation, survival and |
| | | | | differentiation |
| BLM | −0.38 | 0.001 | 0.038 | Anti-DNA damage |
| CD79A | −0.38 | 0.002 | 0.084 | B-cell signaling and activation |
| PDK1 | −0.37 | <0.001 | 0.03 | Metabolism |
| HMGA1 | −0.34 | 0.002 | 0.083 | Gene transcription and cell growth |
| POLR2A | −0.32 | 0.001 | 0.038 | mRNA transcription |
| EZH2 | −0.29 | 0.004 | 0.094 | Epigenetic regulator |
| HDAC11 | −0.23 | 0.005 | 0.099 | Immune cell activation and |
| | | | | tolerance |

In sum, G9.2-17(IgG4) modulated expression of genes involved in upregulation of anti-apoptotic pathways, cell cycle quiescence, immune evasion, and/or increased proliferation, suggesting that the anti-galectin-9 antibody may synergize to overcome resistance to HMAs.

Patients with Genetic Mutations

Therapeutic benefits were observed in AML and MDS patients with mutations in ABL1, ASXL1 (e.g., C.1926_1927INSG (P.G646WFS*12) 1500 VAF, BCOR (e.g., mutation at R342), BCORL1 (e.g., G1682RFS*4), BRAF (e.g., K601N mutant), CBFBMYH1, CBL (e.g., C.1096-1G>T 59.7% VAF (228X consensus coverage) or C.1096-1G>C 18.6% VAF (113X consensus coverage)), CBLB (AF=53.1% MF3+), CEBPA, CSF3R, CUX1, DDX41 (e.g., C.1574G>A (P.R525H) 26.9% VAF), DNMT3A (e.g., C.2478+1G>A 32.7% VAF, or at W305), EZH2, GATA2, IDH2 (e.g., mutation), JAK2, KMT1A (e.g., rearranged), KRAS (e.g., T58I mutation), MPL, MUTYH (e.g., G396D/splice region variant −LOF, NF1 (e.g., C.3198-3_3200DELINES(7) splice region variant −LOF, NPM1 (e.g., mutation), NRAS (e.g., mutation), NSD1, p53 (e.g., TP53stat mutation), PHF6 (e.g., C.971+1G>C 33% VAF), PMLRARA, PD_1, RAD21 (e.g., splice site C.1621-2A>G mutation), RUNX1 (e.g., C.601C>T (P.R201*) 18.4% VAF), RUNX1T1; SETBP1 (e.g., G870S/missense variant-GOF and/or AF=9.1%), SF3B1, SRFS2, SRSF2 (e.g., mutation), STAG2 (e.g., E403SFS*8), TBL1XR1/MECOM fusion, TET2 (e.g., mutation), U2AF1 (e.g., mutation of S34F), WT1, and/or ZRSR2.

Further, correlation analysis using mass cytometry (cytometry by time of flight (CyTOF)) demonstrated robust correlation of various types of immunological cells associated with response upon treatment. For example, immune cells such as basophils, pDC, CD8+ and CD4+ T cells (e.g., CD8+ Naive (CD45RA+CD45RO−) >Ki-67; CD4+ Naive (CD45RA+, CD45RO−) >HLA-DR+CD38+; CD4+ Naive (CD45RA+, CD45RO−) >Ki-67+; CD4+ Naive (CD45RA+, CD45RO−) >HLA-DR+; and CD8+ Naive (CD45RA+CD45RO−) >TIGIT+) showed negative association, while classical monocytes (CD11b+) showed positive association. These results indicate that the anti-Gal9 treatment may be effective in modulating immune responses, thereby benefiting therapeutic efficacy.

### EQUIVALENTS

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

While several inventive embodiments have been described and illustrated herein, those of ordinary skill in the art are readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art are readily appreciate that all parameters, dimensions, materials, and configurations described herein are meant to be exemplary and that the actual parameters, dimensions, materials, and/or configurations depend upon the specific application or applications for which the inventive teachings is/are used. Those skilled in the art are recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/of” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” are refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A method for treating a hematologic malignancy in a subject, comprising:
(a) administering to the subject an antibody that binds human galectin-9 (anti-Gal9 antibody); and
(b) administering to the subject (i) venetoclax, (ii) a hypomethylating agent (HMA), or a combination thereof,
wherein the anti-Gal9 antibody comprises:
(a) a light chain comprising a light chain variable region (V_{L}), which comprises a light chain (LC) complementarity determining region 1 (CDR1) comprising the amino acid sequence of SEQ ID NO: 1, a LC complementarity determining region 2 (CDR2) comprising the amino acid sequence of SEQ ID NO: 2, and a LC complementarity determining region 3 (CDR3) comprising the amino acid sequence of SEQ ID NO: 3 and
(b) a heavy chain comprising a heavy chain variable region (V_{H}), which comprises a heavy chain (HC) complementarity determining region 1 (CDR1) comprising the amino acid sequence of SEQ ID NO: 4, a HC complementarity determining region 2 (CDR2) comprising the amino acid sequence of SEQ ID NO: 5, and a HC complementarity determining region 3 (CDR3) comprising the amino acid sequence of SEQ ID NO: 6.

2. The method of claim 1, wherein the anti-Gal9 antibody is administered to the subject at a dose of about 2 mg/kg to about 16 mg/kg once every week by intravenous infusion;
optionally about 4 mg/kg to about 16 mg/kg once every week by intravenous infusion or about 7.5 mg/kg to about 16 mg/kg once every week by intravenous infusion.

3. The method of claim 2, wherein the anti-Gal9 antibody is administered to the subject at a dose of about 4 mg/kg, about 7.5 mg/kg, about 12 mg/kg, or about 16 mg/kg once every week by intravenous infusion; optionally wherein the anti-Gal9 antibody is administered to the subject at a dose of 12 mg/kg once every week.

4. The method of claim 1, wherein the subject is a human patient having a leukemia or lymphoma.

5. The method of claim 1, wherein the hematologic malignancy is refractory and/or relapsed.

6. The method of claim 1, wherein the subject is a human patient having acute myeloid leukemia (AML), optionally wherein the human patient has relapsed and/or refractory AML post at least one line of prior therapy.

7. The method of claim 6, wherein the human patient is administered the anti-Gal9 antibody, venetoclax, and the HMA, which is azacytidine or decitabine.

8. The method of claim 7, which comprises one or more of a cycle of 28 days, wherein in each cycle:
the anti-Gal9 antibody is administered to the human patient on Day 1, Day 8, Day 15, and Day 22 at a dose of about 4 mg/kg, about 7.5 mg/kg, about 12 mg/kg, or about 16 mg/kg once every week by intravenous infusion;
the venetoclax is administered to the human patient orally at 100 mg on Day 1, at 200 mg on Day 2, and at 400 mg on each day of Days 3-28; and
the azacytidine at 75 mg/m² subcutaneously or intravenously, optionally subcutaneously, on each of Days 1-7.

9. The method of claim 7, which comprises one or more of a cycle of 28 days, wherein in each cycle:
the anti-Gal9 antibody is administered to the human patient on Day 1, Day 8, Day 15, and Day 22 at a dose of about 4 mg/kg, about 7.5 mg/kg, about 12 mg/kg, or about 16 mg/kg once every week, optionally 12 mg/kg once every week, by intravenous infusion;
the venetoclax is administered to the human patient orally at 100 mg on Day 1, at 200 mg on Day 2, and at 400 mg on each of Days 3-28; and
the decitabine at 20 mg/m2 intravenously on each day of Days 1-5.

10. The method of claim 9, wherein the administration of the decitabine is repeated every 28 days.

11. The method of claim 8, wherein the venetoclax is administered at a reduced dose or is terminated when severe venetoclax-associated toxicity occurs.

12. The method of claim 1, wherein the subject is a human patient having myelodysplastic syndrome (MDS), optionally relapsed or refractory, and high risk or very high risk MDS.

13. The method of claim 12, wherein the human patient has relapsed and/or refractory MDS post at least one line of prior therapy.

14. The method of claim 13, wherein the human patient is administered the anti-Gal9 antibody and the HMA, which is azacytidine or decitabine.

15. The method of claim 14, which comprises one or more of a cycle of 28 days, wherein in each cycle:
the anti-Gal9 antibody is administered to the human patient on Day 1, Day 8, Day 15, and Day 22 at a dose of about 4 mg/kg, about 7.5 mg/kg, about 12 mg/kg, or about 16 mg/kg once every week by intravenous infusion; optionally 12 mg/kg once every week; and
the azacytidine at 75 mg/m² subcutaneously or intravenously, optionally subcutaneously, on each of Days 1-7.

16. The method of claim 14, which comprises one or more of a cycle of 28 days, wherein in each cycle:
the anti-Gal9 antibody is administered to the human patient on Day 1, Day 8, Day 15, and Day 22 at a dose of about 4 mg/kg, about 7.5 mg/kg, about 12 mg/kg, or about 16 mg/kg once every week, optionally 12 mg/kg once every week, by intravenous infusion; and
the decitabine at 20 mg/m2 intravenously on each day of Days 1-5.

17. The method of claim 16, wherein the administration of the decitabine is repeated every 28 days.

18. The method of claim 8, wherein the azacytidine or decitabine is administered at a reduced dose or terminated when drug-related toxicity occurs.

19. The method of claim 1, wherein the subject is free of a treatment comprising an immunosuppressant CYP3A4 substrate, which optionally is tacrolimus, cyclosporine, everolimus, or sirolimus.

20. The method of claim 1, wherein the subject is a human patient having AML or MDS and carries a mutation in at least one of ABL1, ASXL1, BCOR, BCORL1, BRAF, CBFBMYH1, CBL, CBLB, CEBPA, CSF3R, CUX1, DDX41, DNMT3A, EZH2, GATA2, IDH2, JAK2, KMT1A, KRAS, MPL, MUTYH, NF1, NPM1, NRAS, NSD1, p53, PHF6, PMLRARA, PD_1, RAD21, RUNX1, RUNX1T1; SETBP1, SF3B1, SRFS2, SRSF2, STAG2, TBL1XR1/MECOM fusion, TET2, U2AF1, WT1, and ZRSR2.

21. The method of claim 1, wherein the anti-Gal9 antibody comprises the V_{L}, which comprises the amino acid sequence of SEQ ID NO:8, and the V_{H}, which comprises the amino acid sequence of SEQ ID NO: 7.

22. The method of claim 1, wherein the anti-Gal9 antibody is an IgG1 molecule or an IgG4 molecule.

23. The method of claim 22, wherein the anti-Gal9 antibody is an IgG4 molecule comprising a modified Fc region relative to the wild-type human IgG4 counterpart.

24. The method of claim 23, wherein the modified Fc region comprises the amino acid sequence of SEQ ID NO: 14.

25. The method of claim 24, wherein the anti-Gal9 antibody comprises a heavy chain, which comprises the amino acid sequence of SEQ ID NO: 19, and a light chain, which comprises the amino acid sequence of SEQ ID NO: 15.

26. The method of claim 1, further comprising monitoring occurrence of an adverse event in the course of the treatment.

27. The method of claim 26, further comprising reducing the dose of the anti-Gal9 antibody in the subject who has occurrence of the adverse event occurs.
